(19) United States
(12) Patent Application Publication
(10) Pub. No.: US 2020/0060518 A1
(43) Pub. Date: Feb. 27, 2020

(54) Fitting For A Medical Scoping Device Having A Plurality of Protuberances

(71) Applicant: Madivators Inc., Minneapolis, MN (US)

(72) Inventors: Suranjan Roychowdhury, Plymouth, MN (US); John Schreiner, St. Louis, MN (US)

(21) Appl. No.: 16/610,649

(22) PCT Filed: May 8, 2018
(86) PCT No.: PCT/US2018/031595

§ 371 Date: Nov. 4, 2019

Related U.S. Application Data
(60) Provisional application No. 62/504,303, filed on May 10, 2017.

(51) Int. Cl. A61B 1/00

(52) U.S. Cl. CPC A61B 1/00085; A61B 1/00101; A61B 1/00103

(57) ABSTRACT

Provided is a fitting for a medical scoping device, the fitting comprising a body defining a longitudinal axis, the body having an interior having an opening to receive an endoscope along the longitudinal axis, the body having a cylindrical portion comprising protuberances being spaced apart and circumferentially arrayed with respect to one another and extending from the cylindrical portion, each of the protuberances having an outer edge to engage tissue.

### CROSS REFERENCE TO RELATED APPLICATIONS

This application claims priority to U.S. Provisional Patent Application Ser. No. 62/504,303 filed May 10, 2017.

### TECHNICAL FIELD

The present disclosure relates to fittings for a medical scoping device that improve visibility and/or reduce the risk of complications during a procedure.

### BACKGROUND

Endoscopes play a critical role in medical diagnosis and treatment. Often, endoscopes can be used to illuminate, examine and document difficult-to-access areas and other body regions to facilitate diagnosis and treatment of hidden diseases. Endoscopes can also assist in enhancing the planning and preparation of invasive operations. Endoscopes include cameras to allow for real-time visualization of inner biological lumens, such as that of the esophagus, the stomach, the duodenum, the small intestine, the colon, and the entire length of the large intestine with various types of endoscopes.

There are various types of endoscopic procedures. For example, enteroscopy is the endoscopic examination of the small intestine. Colonoscopy is the endoscopic examination of the colon and the distal part of the small bowel. Flexible sigmoidoscopy is the examination of the rectum and lower part of the bowel. These forms of endoscopy allow for visual diagnosis of the digestive tract and aid in biopsy or removal of suspected lesions. Although endoscopic examinations are effective techniques for assessing the state of health of the bowel, they are inconvenient, uncomfortable, expensive and are associated with potential complications.

The inner lumen of the colon is composed of folds and undulations. As the endoscope is advanced into the lumen, the folds can hamper the medical practitioner's ability to visualize the entire surface of the mucosa and in particular, detect pre-malignant and malignant lesions tucked between the folds during extubation. Furthermore, the position of the tip of the endoscope may be difficult to maintain from the moment at which a lesion or polyp is detected to the completion of any therapeutic procedure. While a medical practitioner advances or retracts the endoscope, the geometry of the lumen and the folds may cause the tip of the endoscope to jerk and slip, particularly when traversing a bend of the colon or other biological lumen. Should the endoscope slip backwards, the medical practitioner will lose his or her position, and may struggle to find it once again. This is particularly important when a lesion, a cancer or a polyp is identified, as the medical practitioner now has to reposition the endoscope to find it again.

Therefore, there is a need for a fitting (e.g., cover, cap, etc.) for a medical scoping device (e.g., endoscope) that reduces the risk of complications during a procedure. A medical scoping device fitting which allows for improved visualization of a biological lumen (e.g., colon, small bowel, etc.) would be beneficial. It would also be beneficial to provide a medical scoping device fitting that expands the aforementioned folds of the biological lumen to improve visibility of the lining of the biological lumen. Moreover, it would be beneficial to provide a disposable medical scoping device fitting that is compressible to allow access into narrower portions of a biological lumen and debris from the lumen to pass by the fitting.

### SUMMARY

In some embodiments, a fitting for a medical scoping device is provided that reduces the risk of complications during a procedure. A medical scoping device fitting is provided, which allows for improved visualization of a biological lumen (e.g., colon, small bowel, etc.). In some embodiments, there is a medical scoping device fitting that expands the folds of the biological lumen to improve visibility of the lining of the biological lumen. In some embodiments, there is a disposable medical scoping device fitting that is compressible to allow access into narrower portions of a biological lumen and debris from the lumen to pass by the fitting.

In one embodiment, there is a fitting for a medical scoping device, the fitting comprising a body defining a longitudinal axis, the body having first and second regions, and an interior having an opening to receive the medical scoping device along the longitudinal axis, each of the first and second regions of the body comprising protuberances, each protuberance having an inner end and an outer edge to engage tissue, each protuberance being spaced apart and radially or circumferentially arrayed with respect to one another and extending from the body of the fitting, wherein each protuberance has varying flexibility from the inner end to the outer edge of each protuberance.

In another embodiment, there is a fitting for an endoscope, the fitting comprising a body defining a longitudinal axis, the body having an interior having an opening to receive an endoscope along the longitudinal axis, the body having a first cylindrical portion and a second cylindrical portion separable from the first cylindrical portion, the first cylindrical portion comprising protuberances being spaced apart and radially or circumferentially arrayed with respect to one another and extending from the first cylindrical portion, the second cylindrical portion comprising protuberances being spaced apart and radially or circumferentially arrayed with respect to one another and extending from the second cylindrical portion, each of the protuberances having an inner end and an outer edge to engage tissue, wherein each protuberance has varying flexibility from the inner end to the outer edge of each protuberance.

In one exemplary embodiment, there is a method of performing an endoscopy, the method comprising disposing an endoscope cap on a distal end of an endoscope, the cap comprising a body defining a longitudinal axis, the body having first and second regions, and an interior having an opening to receive the endoscope along the longitudinal axis, each of the first and second regions of the body comprising protuberances, each protuberance having an inner end and an outer edge to engage tissue, each protuberance being spaced apart and radially or circumferentially arrayed with respect to one another and extending from the body of the endoscope, wherein each protuberance has varying flexibility from the inner end to the outer edge of each protuberance; and inserting the distal end of the endoscope into a biological lumen to move the protuberances radially inward relative to the body of the cap; and moving the endoscope proximally in the biological lumen for a distance to move the protuberances radially outward relative to the body of the cap.

In another exemplary embodiment, there is a kit for performing an endoscopy, the kit comprising a disposable endoscope cap, the cap having a body defining a longitudinal axis, the body having first and second regions, and an interior having an opening to receive an endoscope along the longitudinal axis, each of the first and second regions of the body comprising protuberances, each protuberance having an inner end and an outer edge to engage tissue, each protuberance being spaced apart and radially or circumferentially arrayed with respect to one another and extending from the body of the cap, wherein each protuberance has varying flexibility from the inner end to the outer edge of each protuberance; and a sterilized packaging configured to provide an airtight seal for the cap.

In yet another embodiment, there is a fitting for an endoscope, the fitting comprising a body defining a longitudinal axis, the body having an exterior surface, and an interior having an opening to receive the endoscope along the longitudinal axis, the exterior surface of the body comprising protuberances, each protuberance having an inner end, a middle portion, and an outer edge to engage tissue, each protuberance being spaced apart and radially or circumferentially arrayed with respect to one another and extending from the body of the fitting, wherein the middle portion comprises a recess that increases flexibility of the outer edge relative to the inner end of each protuberance.

In one embodiment, there is a fitting for an endoscope, the fitting comprising a body defining a longitudinal axis, the body having an exterior surface, and an interior having an opening to receive the endoscope along the longitudinal axis, the exterior surface of the body comprising protuberances, each protuberance having an inner end, a middle portion, and an outer edge to engage tissue, each protuberance being spaced apart and radially or circumferentially arrayed with respect to one another and extending from the body of the fitting, wherein (i) the inner end of each protuberance has the same or increased width or surface area relative to the outer edge of each protuberance and the middle portion has a decreased width or surface area relative to the width of the inner end or outer edge of the protuberance; (ii) the inner end of each protuberance has a reinforced region having increased thickness relative to the thickness of the middle portion and the outer edge of the protuberance; or (iii) the inner end of each protuberance has decreased width or surface area relative to the outer edge of each protuberance.

In another embodiment, there is a fitting for an endoscope, the fitting comprising a body defining a longitudinal axis, the body having an exterior surface, and an interior having an opening to receive the endoscope along the longitudinal axis, the exterior surface of the body comprising protuberances, each protuberance having an inner end, a middle portion, and an outer edge to engage tissue, each protuberance being spaced apart and radially or circumferentially arrayed with respect to one another and extending from the body of the fitting, wherein the outer edge of each protuberance comprises a raised surface configured to engage tissue, wherein said engagement causes a change in shape of the fitting.

In some embodiments, there is a fitting for an endoscope, the fitting comprising a body defining a longitudinal axis, the body having an interior having an opening to receive an endoscope along the longitudinal axis, the body having a cylindrical portion comprising protuberances being spaced apart and circumferentially arrayed with respect to one another and extending from the cylindrical portion, each of the protuberances having an outer edge to engage tissue, wherein (i) the interior comprises a plurality of raised surfaces disposed thereon; and/or (ii) each protuberance having a bottom surface having a plurality of raised surfaces disposed thereon.

In some embodiments, there is a fitting for an endoscope, the fitting comprising a body defining a longitudinal axis, the body having an interior having an opening to receive an endoscope along the longitudinal axis, the interior having a plurality of ribs extending longitudinally in the interior, the body having a cylindrical portion comprising protuberances being spaced apart and circumferentially arrayed with respect to one another and extending from the cylindrical portion, each of the protuberances having an inner end and an outer edge to engage tissue, wherein the inner end of each protuberance comprises a reinforced region.

In some embodiments, there is a method of performing an endoscopy, the method comprising disposing an endoscope cap on a distal end of an endoscope, the cap comprising a body defining a longitudinal axis, the body having an interior having an opening to receive an endoscope along the longitudinal axis, the body having a cylindrical portion comprising protuberances being spaced apart and circumferentially arrayed with respect to one another and extending from the cylindrical portion, each of the protuberances having an outer edge to engage tissue, wherein (i) the interior comprises a plurality of raised surfaces disposed thereon; and/or (ii) each protuberance having a bottom surface having a plurality of raised surfaces disposed thereon; and inserting the distal end of the endoscope into a biological lumen to move the protuberances radially inward relative to the body of the cap; and moving the endoscope proximally in the biological lumen for a distance to move the protuberances radially outward relative to the body of the cap.

In one embodiment, there is a fitting for a medical scoping device, the fitting comprising an elongated body defining a longitudinal axis, the body having an interior having an opening to receive an endoscope along the longitudinal axis, the interior having a plurality of ribs extending longitudinally in the interior, the body having a cylindrical portion comprising elongated flexible protuberances being spaced apart and circumferentially arrayed with respect to one another and extending from the cylindrical portion, each elongated flexible protuberance having a bottom surface to engage tissue.

In another embodiment, the fitting comprises a tab extending from a proximal end of the fitting, the fitting and/or tab having a perforation line disposed on the elongated body and aligned along the longitudinal axis, the perforation line configured to facilitate tearing to remove the fitting from the endoscope.

In yet another embodiment, the fitting comprises a tab comprising a first wall and a second wall disposed along the elongated body and the longitudinal axis, the first wall and the second wall having a thickness that is less than a thickness of the body, and the tab having a projection extending outwardly from a proximal end of the fitting, wherein the tab is peeled off the body to facilitate removal of the fitting from the endoscope.

In some embodiments, the fitting or tab comprises a wall disposed along the elongated body and the longitudinal axis, the wall having a thickness that is less than a thickness of the body to facilitate removal of the fitting from the endoscope.

In some embodiments, the fitting or tab comprises a first wall and a second wall disposed along the elongated body and the longitudinal axis, the first wall and the second wall having a thickness that is less than a thickness of the body to facilitate removal of the fitting from the endoscope.

In one embodiment, the fitting further comprises a tube disposed on a distal end of the body. In one embodiment, the tube is transparent and is fused to the distal end of the body. In another embodiment, the tube is a clear plastic material.

In one embodiment, there is a fitting for a medical scoping device, the fitting comprising an elongated body defining a longitudinal axis, the body having an interior having an opening to receive an endoscope along the longitudinal axis, the interior having a plurality of ribs extending longitudinally in the interior, the body having a cylindrical portion comprising elongated flexible protuberances being spaced apart and circumferentially arrayed with respect to one another and extending from the cylindrical portion, each protuberance having a bottom surface having a first raised surface and a second raised surface disposed on a distal end.

In one embodiment, there is a fitting for a medical scoping device, the fitting comprising an elongated body defining a longitudinal axis, the body having an interior having an opening to receive an endoscope along the longitudinal axis, the interior having a plurality of ribs extending longitudinally in the interior, the body having a cylindrical portion comprising elongated and narrow flexible protuberances being spaced apart and circumferentially arrayed with respect to one another and extending from the cylindrical portion, each protuberance having a smooth bottom surface to engage tissue.

In one embodiment, there is a fitting for a medical scoping device, the fitting comprising an elongated body defining a longitudinal axis, the body having an interior having an opening to receive an endoscope along the longitudinal axis, the body having a cylindrical portion, the cylindrical portion comprising a first set of protuberances being spaced apart and circumferentially arrayed with respect to one another and extending from the cylindrical portion, the first set of protuberances each comprising a bottom surface having a first raised surface and a second raised surface disposed on a distal end, an inner end and an outer edge to engage tissue, the elongated body further comprising a second set of protuberances being spaced apart and circumferentially arrayed with respect to one another and extending from the elongated body, each of the second set of protuberances having an inner end and an outer edge to engage tissue, and a window disposed between the outer edge and the inner edge of the second set of protuberances.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a perspective view of an embodiment of a medical scoping device fitting (e.g., endoscope) in accordance with the principles of the present disclosure. The fitting illustrated includes two rows of protuberances having central windows and recesses or notches to allow for greater flexibility of the outer edge of the protuberances;

FIG. 2 illustrates a side view of the fitting shown in FIG. 1;

FIG. 3 illustrates a side cross-sectional view of another embodiment of a fitting, where the protuberances are contoured at the outer edges to reduce tissue damage and the outer edges of the protuberances in the first row have a decreased surface area relative to the protuberances in the second row;

FIG. 4 illustrates a side cross-sectional view of another embodiment of a fitting wherein the protuberances include a reinforced portion in the inner end;

FIG. 4A illustrates a side view of the fitting shown in FIG. 4 wherein the protuberances are being flexed outward, which often occurs when the medical scoping device tip is moved proximally out of the biological lumen;

FIG. 4B illustrates a magnified side view of the fitting shown in FIG. 4 wherein the protuberances have an outer edge and an incline adjacent the outer edge, which reduces damage to tissue and allows additional flexibility at the outer edge;

FIG. 4C illustrates a side view of the fitting shown in FIG. 3 wherein the protuberances are being flexed outward, which often occurs when the medical scoping device tip is moved proximally out of the biological lumen;

FIG. 5 illustrates a top view of another embodiment of the fitting having longer and thinner protuberances;

FIG. 6 illustrates a perspective view of an embodiment of a medical scoping device fitting shown in FIG. 5 in accordance with the principles of the present disclosure. The fitting illustrated includes two rows of protuberances having central windows and thinned outer portions to customize or vary flexibility;

FIG. 7 illustrates a side view of the fitting shown in FIG. 5, the protuberances are in the resting position;

FIG. 8 illustrates a top view of the fitting shown in FIG. 5;

FIG. 9 illustrates a perspective view of an embodiment of a medical scoping device fitting in accordance with the principles of the present disclosure. The fitting illustrated is monolithic and includes two rows of protuberances having central windows, the outer edge of the fitting having a reduced surface area relative to the inner edge of the protuberance;

FIG. 10 illustrates a side view of the fitting shown in FIG. 9;

FIG. 11 illustrates a bottom view of the fitting shown in FIG. 9. The fitting illustrated includes an overmolded portion to facilitate proper positioning on an endoscope, the protuberances are shown circumferentially arrayed outwardly from the body;

FIG. 12 illustrates a perspective view of an embodiment of a medical scoping device fitting in accordance with the principles of the present disclosure. The fitting illustrated includes two rows of protuberances having wide inner ends having increased surface area and width when compared to the reduced surface area and width of the outer edge of the protuberances;

FIG. 13 illustrates a side view of the fitting shown in FIG. 12, the notches or recesses shown in this view increase flexibility of the outer edges of the protuberances;

FIG. 14 illustrates a top view of the fitting shown in FIG. 12;

FIG. 15 illustrates a perspective view of an embodiment of a medical scoping device fitting in accordance with the principles of the present disclosure. The fitting illustrated includes two rows of protuberances having wide inner ends, narrow middle portions, and flared outer ends;

FIG. 16 illustrates a side view of the fitting shown in FIG. 15, the notches or recesses shown in this view increase flexibility of the outer edges of the protuberances;

FIG. 17A illustrates a top view of the fitting shown in FIG. 15;

FIG. 17B illustrates a bottom view of the fitting shown in FIG. 15, the notches or recesses shown in this view increase flexibility of the outer edges of the protuberances and the protuberances span slightly less than 360 degrees around the body of the fitting;

FIG. 18 illustrates a perspective view of an embodiment of a medical scoping device fitting in accordance with the principles of the present disclosure. The fitting illustrated includes two rows of protuberances having linearly widening profiles;

FIG. 19 illustrates a side view of the fitting shown in FIG. 18. The fitting illustrated includes two rows of protuberances having wide outer edges having increased surface area and width when compared to the reduced surface area and width of the inner end of the protuberances, the notches or recesses shown in this view increase flexibility of the outer edges of the protuberances;

FIG. 20 illustrates a top view of the fitting shown in FIG. 18 the protuberances span slightly less than 360 degrees around the body of the fitting;

FIG. 21 illustrates a perspective view of an embodiment of a medical scoping device fitting in accordance with the principles of the present disclosure. The fitting illustrated includes a row of protuberances as well as a transparent or semi-transparent extension member;

FIG. 22 illustrates a side view of the fitting shown in FIG. 21;

FIG. 23 illustrates a top view of the fitting shown in FIG. 21;

FIG. 24 illustrates a side view of an embodiment of a medical scoping device fitting in accordance with the principles of the present disclosure. The protuberances of the fitting comprise a plurality of raised surfaces at the outer edges to aid in frictional force to move the folds of the biological lumen;

FIG. 24A illustrates a perspective view of the fitting shown in FIG. 24 attached to the distal end of a medical scoping device. The fitting includes compressible members which are in a low profile configuration to allow passage through a biological lumen;

FIG. 24B illustrates a perspective view of the fitting shown in FIG. 24 attached to the distal end of a medical scoping device. The fitting includes compressible members which are in an expanded configuration to increase the inner diameter of a biological lumen;

FIG. 25 illustrates a bottom view of the fitting shown in FIG. 24;

FIG. 26 illustrates a top view of the fitting shown in FIG. 24;

FIG. 27 illustrates a schematic anatomical section of a medical scoping device fitting of the present application in the course of a medical scoping procedure. FIG. 27 shows insertion of the scoping device into the colon of an individual undergoing an endoscopic procedure. The protuberances of the fitting move radially inward as the fitting enters the colon and the protuberances are compressed by the colon wall;

FIG. 28 illustrates a schematic anatomical section of a medical scoping device fitting of the present application shown in FIG. 27 in the course of a medical scoping procedure, where the protuberance moves radially outward as the medical scoping device is withdrawn from the colon and protuberances unfold the colon lining to improve visualization of the colon lining, alternatively this can be accomplished by air suction causing the colon wall to collapse or wrap around the fitting;

FIG. 29A illustrates a perspective view of an embodiment of a medical scoping device fitting in accordance with the principles of the present disclosure. The fitting illustrated is a two component system, where two rows of protuberances are present and the fitting can be simply assembled;

FIG. 29B illustrates a perspective view of a partially assembled medical scoping device fitting shown in FIG. 29A;

FIG. 29C illustrates a perspective view of an assembled medical scoping device fitting shown in FIG. 29A;

FIG. 30A illustrates a perspective view of an embodiment of a medical scoping device fitting in accordance with the principles of the present disclosure. The fitting illustrated includes a row of protuberances having dual protuberance lengths, a cylindrical body with various shapes of cavities on exterior walls and inner walls having ribs and a pattern of raised surfaces between two ribs;

FIG. 30B illustrates a side view of the fitting shown in FIG. 30A;

FIG. 30C illustrates a top view of another embodiment of the fitting having long and short protuberances;

FIG. 31A illustrates a perspective view of an embodiment of a medical scoping device fitting (e.g., endoscope) in accordance with the principles of the present disclosure. The fitting illustrated includes a row of protuberances, a body member with cavities on exterior walls and inner walls having ribs and a pattern of raised surfaces between two ribs;

FIG. 31B illustrates a side view of the fitting shown in FIG. 31A;

FIG. 31C illustrates a top view of the fitting shown in FIG. 31A;

FIG. 32A illustrates a perspective view of an embodiment of a medical scoping device fitting (e.g., endoscope) in accordance with the principles of the present disclosure. The fitting illustrated includes a row of protuberances having a pattern of raised surfaces extending perpendicularly from each protuberance, a body member with cavities on exterior walls and inner walls having ribs and a pattern of raised surfaces between two ribs;

FIG. 32B illustrates a side view of the fitting shown in FIG. 32A;

FIG. 32C illustrates a top view of the fitting shown in FIG. 32A;

FIG. 33A illustrates a perspective view of an embodiment of a medical scoping device fitting (e.g., endoscope) in accordance with the principles of the present disclosure. The fitting illustrated includes a row of protuberances with reinforced regions at the inner end of protuberance having a pattern of raised surfaces extending perpendicularly from each protuberance, a body member with cavities on exterior walls and inner walls having ribs and a pattern of raised surfaces between two ribs;

FIG. 33B illustrates a side view of the fitting shown in FIG. 33A;

FIG. 33C illustrates a top view of the fitting shown in FIG. 33A;

FIG. 34A illustrates a perspective view of an embodiment of a medical scoping device fitting (e.g., endoscope) in accordance with the principles of the present disclosure. The fitting illustrated includes a row of protuberances with reinforced regions at the inner end of protuberances having a pattern of raised surfaces extending perpendicularly from each protuberance, a row of protuberances having a wide outer edge that tapers to an inner end that is narrower relative to the outer edge, central windows, and sloped portions at the outer edge away from the body member, a body member with cavities on exterior walls and inner walls having ribs and a pattern of raised surfaces between two ribs;

FIG. 34B illustrates a side view of the fitting shown in FIG. 34A;

FIG. 34C illustrates a top view of the fitting shown in FIG. 34A;

FIG. 35 illustrates a perspective view of an embodiment of a medical scoping device fitting (e.g., endoscope) in accordance with the principles of the present disclosure. The fitting illustrated includes a row of protuberances with reinforced regions in shapes of bubbles at the inner end of protuberances having sloped portions at the outer edge away from the body member and inner walls having ribs and a pattern of raised surfaces between two ribs;

FIG. 36 illustrates a perspective view of an embodiment of a medical scoping device fitting (e.g., endoscope) in accordance with the principles of the present disclosure. The fitting illustrated includes a row of protuberances with reinforced regions at the inner end of protuberances having a narrowed portion near the inner end of protuberances and a wider and thicker portion of the distal end and the proximal end than the cylindrical body member;

FIG. 37 illustrates a perspective view of an embodiment of a medical scoping device fitting (e.g., endoscope) in accordance with the principles of the present disclosure. The fitting illustrated includes a row of protuberances with reinforced regions at the inner end of protuberances having a pattern of raised surfaces extending perpendicularly from each protuberance, a wider and thicker portion of the distal end than the cylindrical body member, a wide portion of the cylindrical body and inner walls having ribs and a pattern of raised surfaces between two ribs;

FIG. 38A illustrates a perspective view of an embodiment of a medical scoping device fitting (e.g., endoscope) in accordance with the principles of the present disclosure. The fitting illustrated includes an elongated body defining a longitudinal axis, the body having an interior having an opening to receive an endoscope along the longitudinal axis, the interior having a plurality of ribs extending longitudinally in the interior, the body having a cylindrical portion comprising elongated flexible protuberances being spaced apart and circumferentially arrayed with respect to one another and extending from the cylindrical portion, each elongated flexible protuberance having a bottom surface to engage tissue;

FIG. 38B illustrates a side view of the fitting shown in FIG. 38A;

FIG. 38C illustrates a top view of the fitting shown in FIG. 38A;

FIG. 38D illustrates a perspective view of the fitting shown in FIG. 38A defining a tab;

FIG. 38E illustrates a side view of the fitting shown in FIG. 38D;

FIG. 38F illustrates a side view of the fitting shown in FIG. 38D;

FIG. 38G illustrates a side view of the fitting shown in FIG. 38D defining a first tab and a second tab;

FIG. 38H illustrates a perspective view of the fitting shown in FIG. 38A defining a tab;

FIG. 381 illustrates a side view of the fitting shown in FIG. 38H;

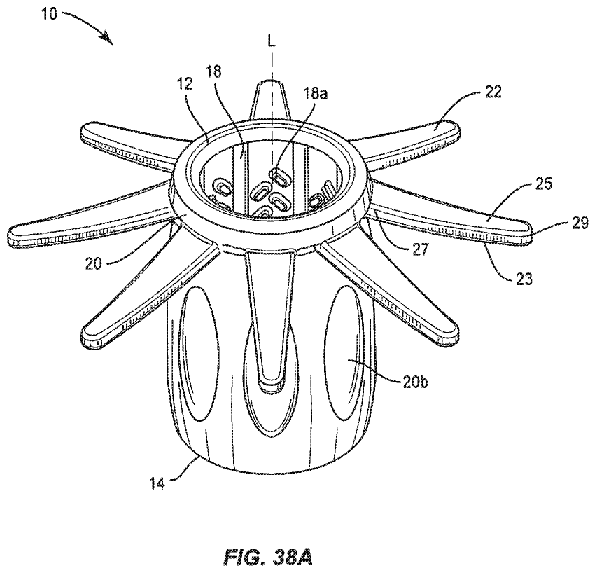

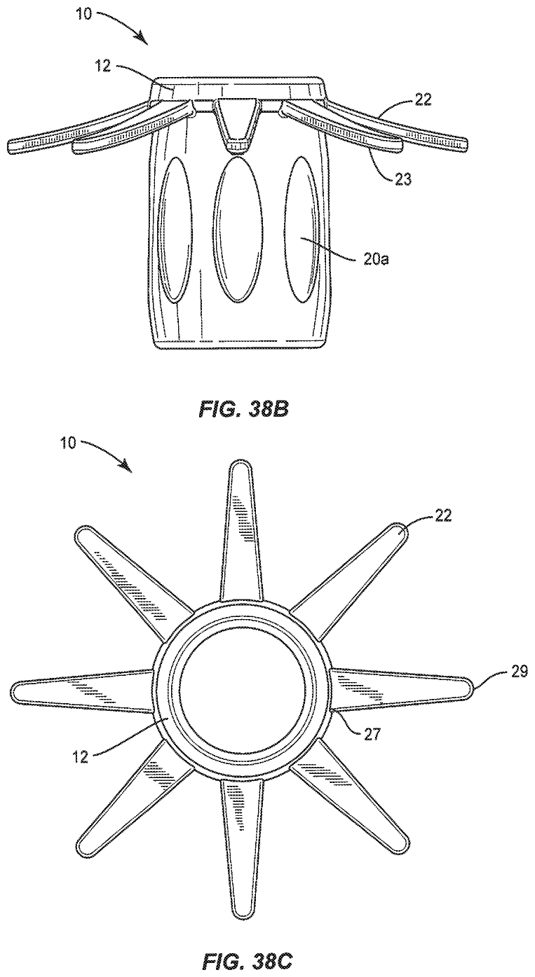

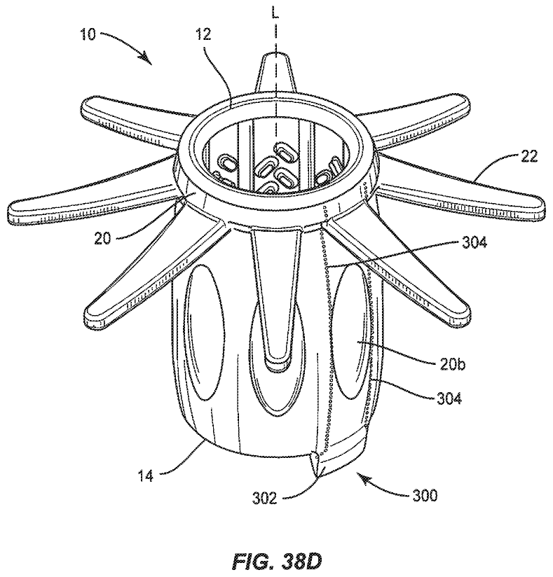

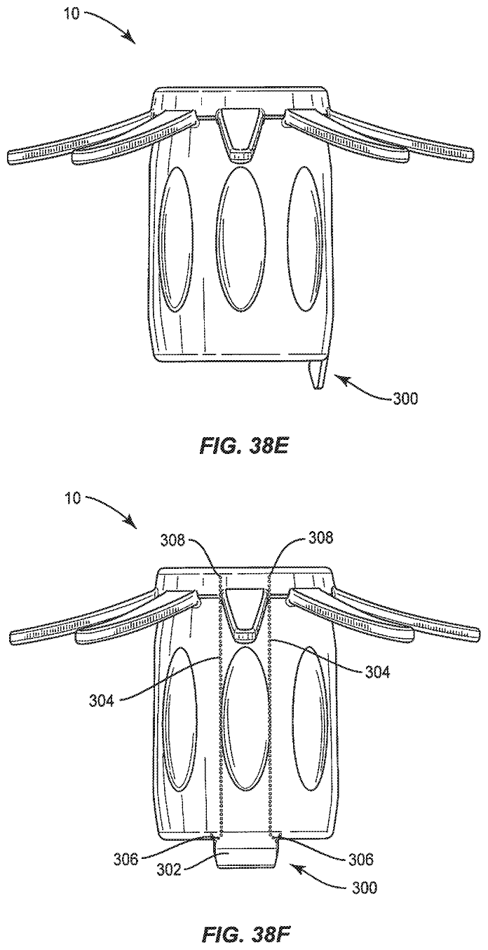

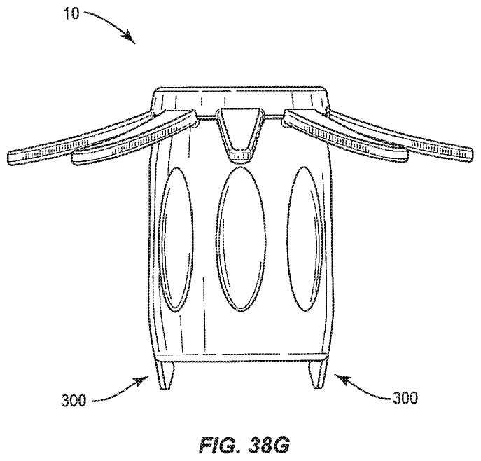

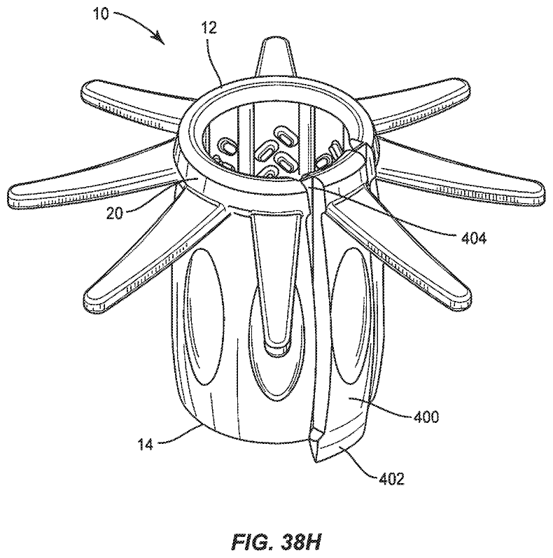

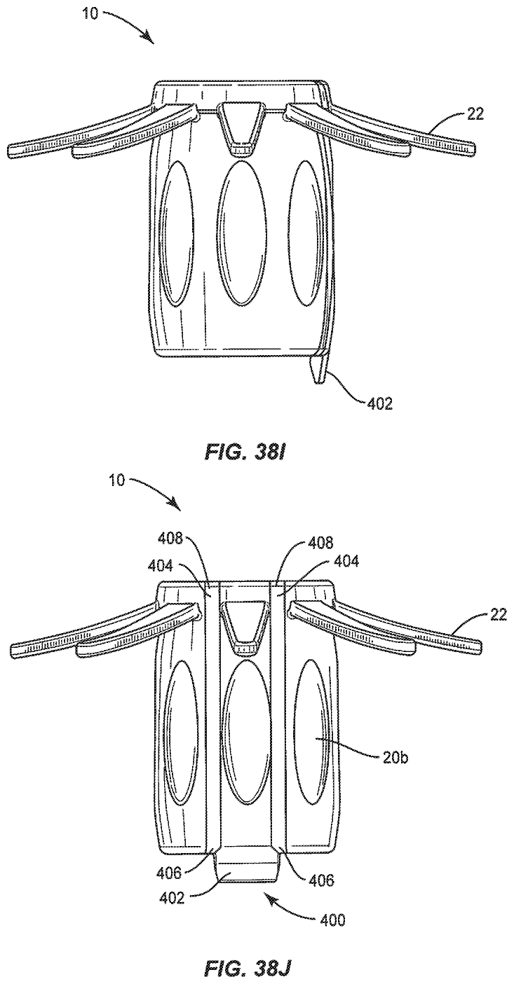

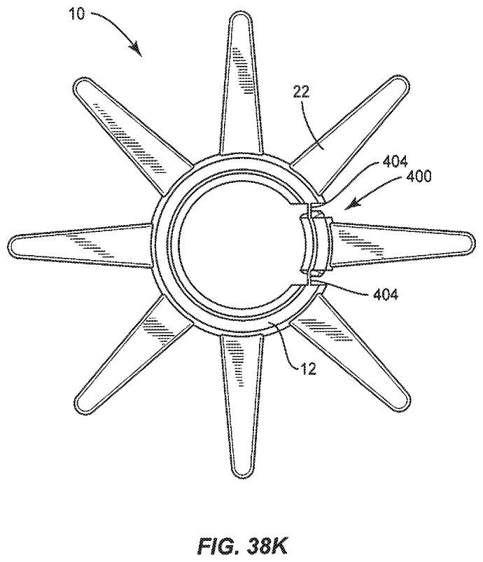

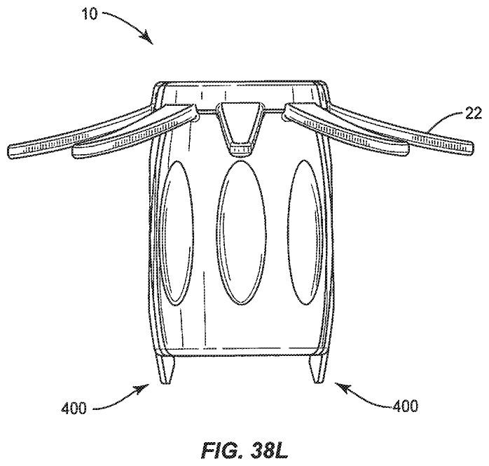

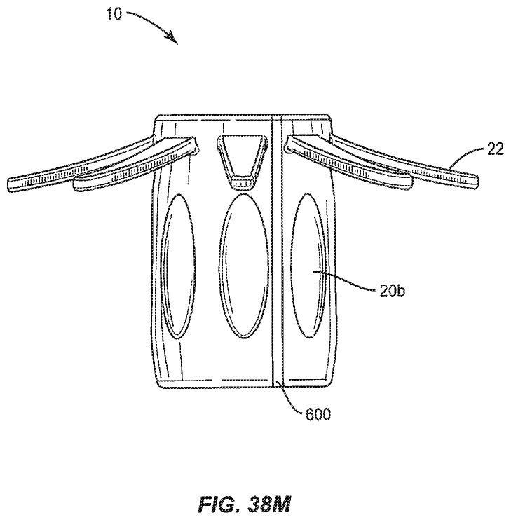

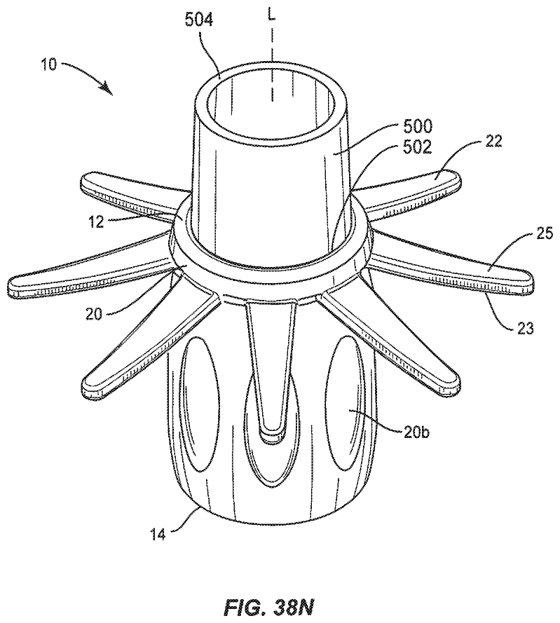

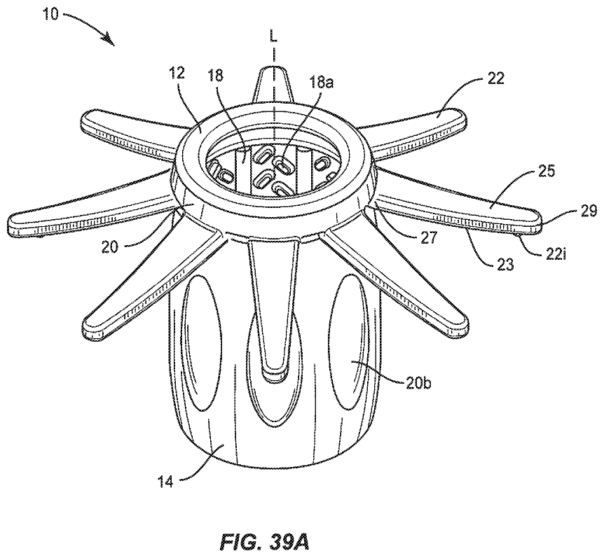

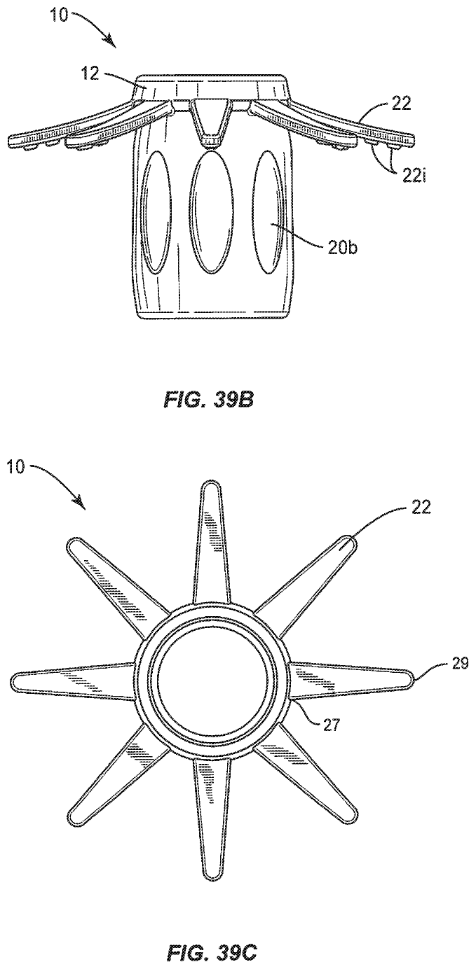

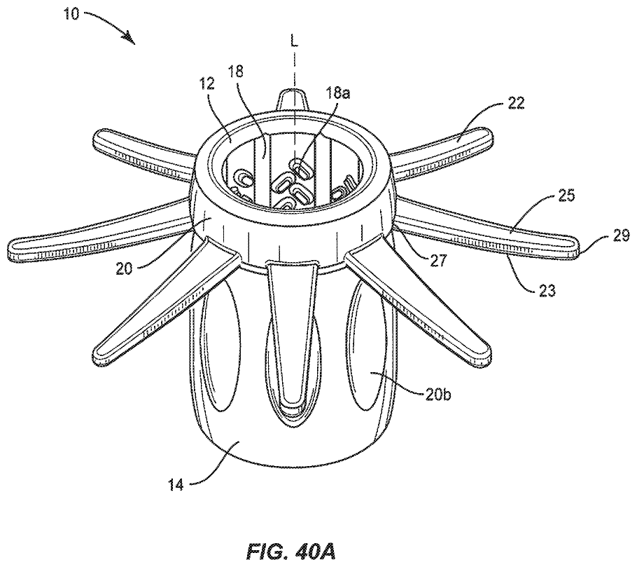

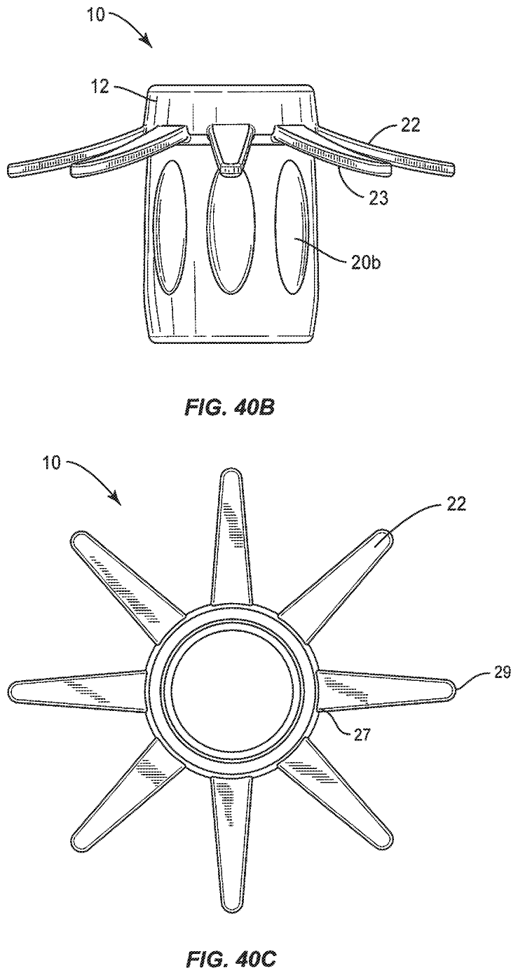

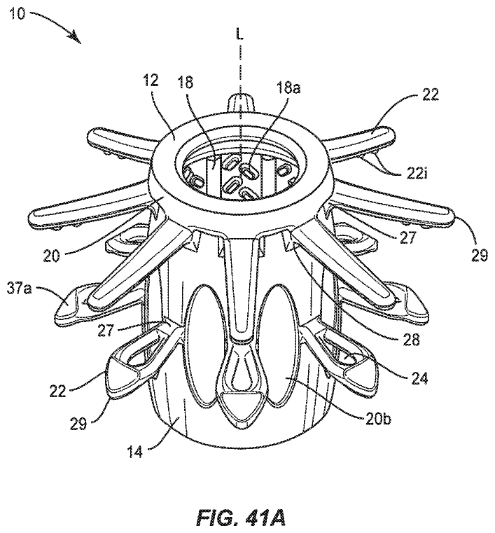

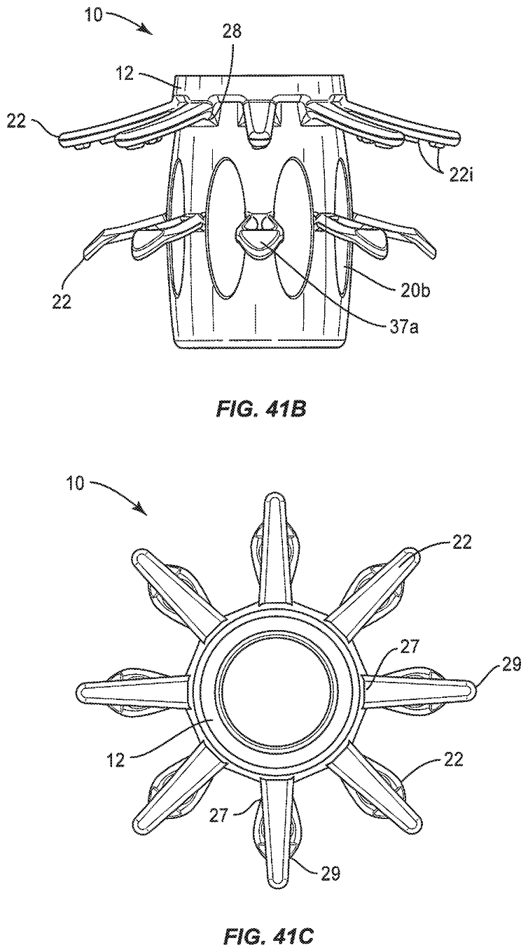

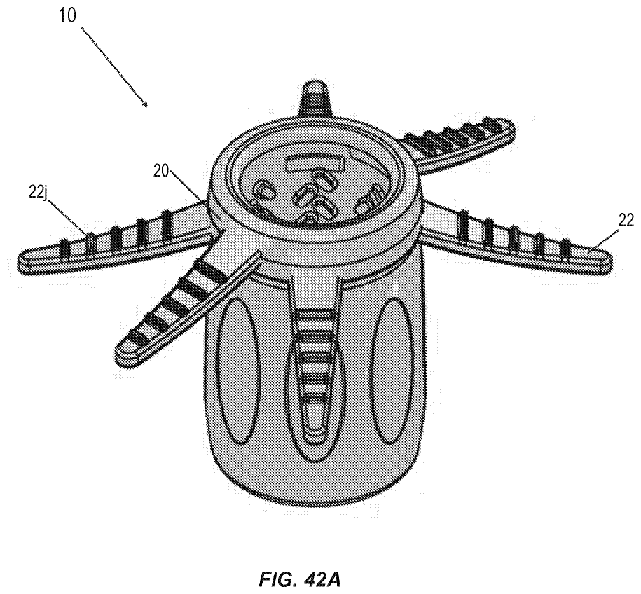

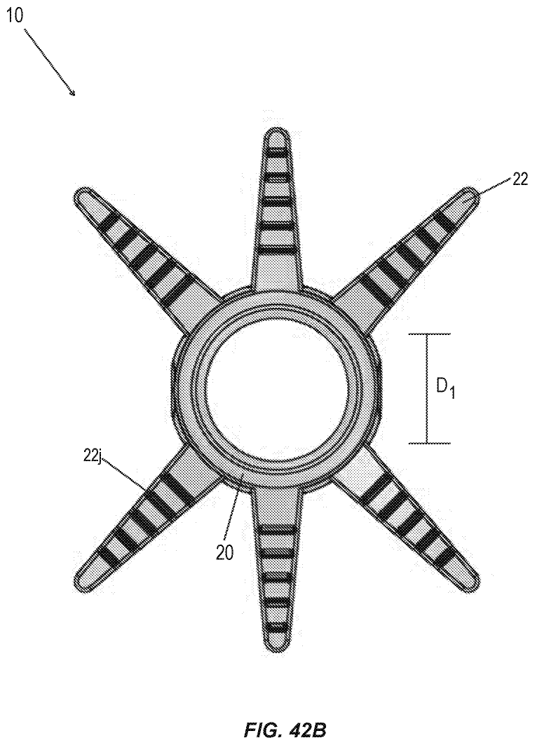

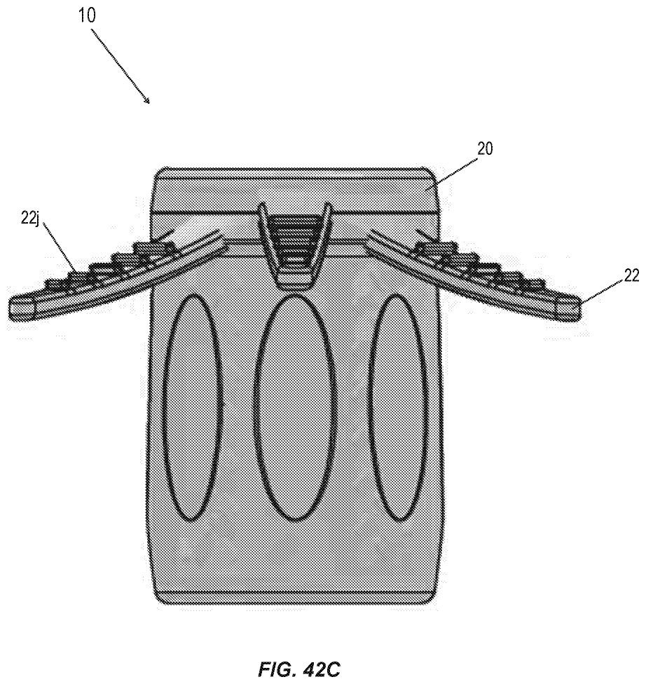

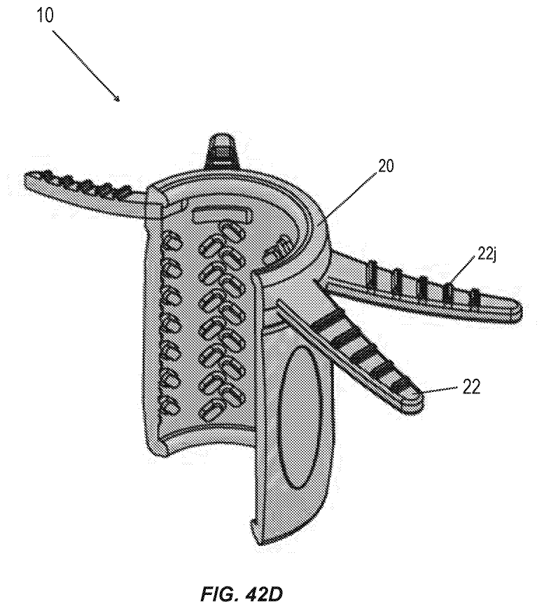

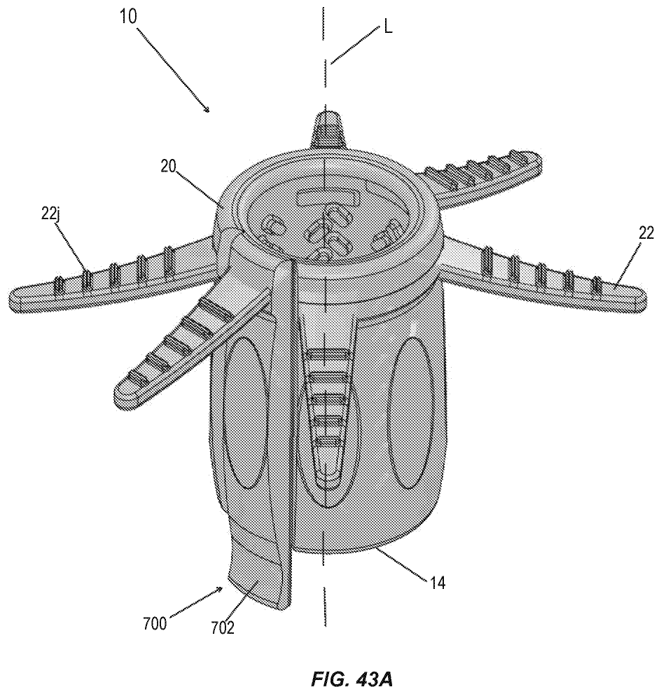

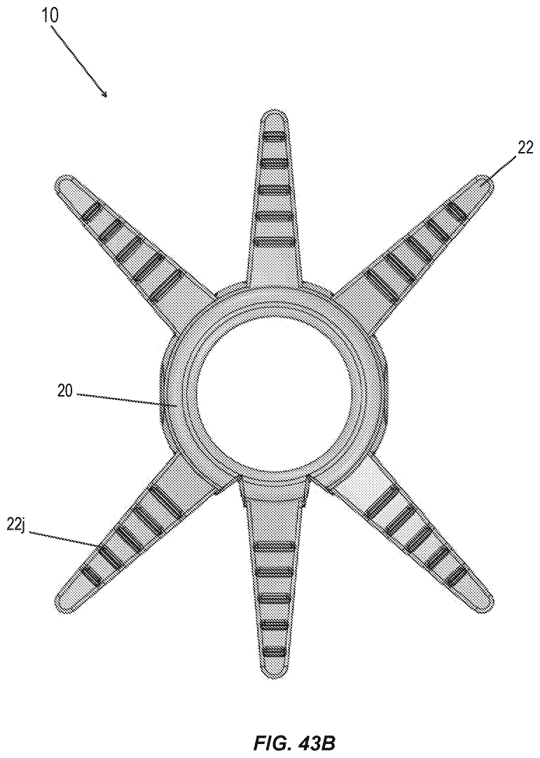

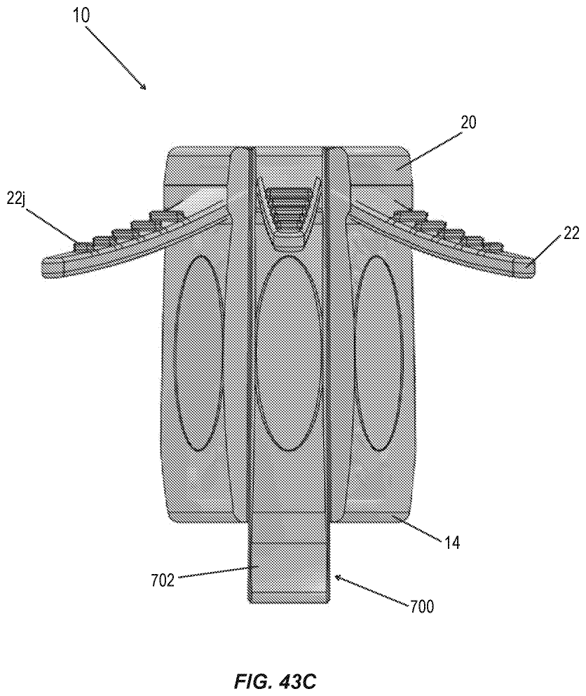

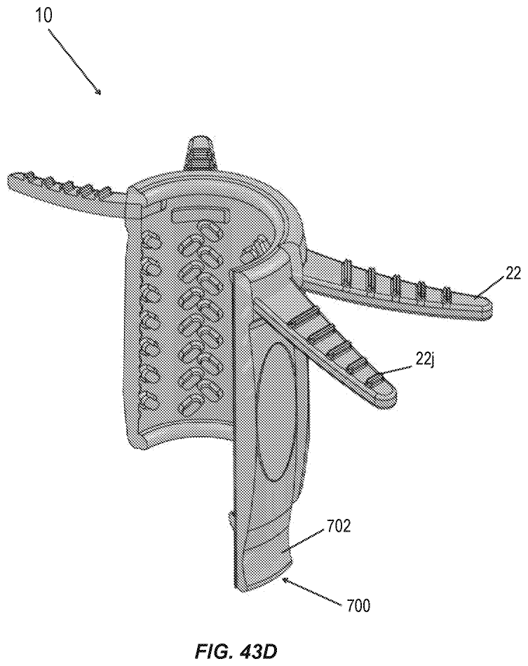

FIG. 38J illustrates a side view of the fitting shown in FIG. 38H;

FIG. 38K illustrates a top view of the fitting shown in FIG. 38H;

FIG. 38L illustrates a side view of the fitting shown in FIG. 38H defining a first tab and a second tab;

FIG. 38M illustrates a side view of the fitting shown in FIG. 38A defining a strip;

FIG. 38N illustrates a perspective view of the fitting shown in FIG. 38A comprising a transparent tube;

FIG. 39A illustrates a perspective view of an embodiment of a medical scoping device fitting (e.g., endoscope) in accordance with the principles of the present disclosure. The fitting illustrated includes an elongated body defining a longitudinal axis, the body having an interior having an opening to receive an endoscope along the longitudinal axis, the interior having a plurality of ribs extending longitudinally in the interior, the body having a cylindrical portion comprising elongated flexible protuberances being spaced apart and circumferentially arrayed with respect to one another and extending from the cylindrical portion, each protuberance having a bottom surface having a first raised surface and a second raised surface disposed on a distal end;

FIG. 39B illustrates a side view of the fitting shown in FIG. 39A;

FIG. 39C illustrates a top view of the fitting shown in FIG. 39A;

FIG. 40A illustrates a perspective view of an embodiment of a medical scoping device fitting (e.g., endoscope) in accordance with the principles of the present disclosure. The fitting illustrated includes an elongated body defining a longitudinal axis, the body having an interior having an opening to receive an endoscope along the longitudinal axis, the interior having a plurality of ribs extending longitudinally in the interior, the body having a cylindrical portion comprising elongated and narrow flexible protuberances being spaced apart and circumferentially arrayed with respect to one another and extending from the cylindrical portion, each protuberance having a smooth bottom surface to engage tissue;

FIG. 40B illustrates a side view of the fitting shown in FIG. 40A;

FIG. 40C illustrates a top view of the fitting shown in FIG. 40A;

FIG. 41A illustrates a perspective view of an embodiment of a medical scoping device fitting (e.g., endoscope) in accordance with the principles of the present disclosure. The fitting illustrated includes an elongated body defining a longitudinal axis, the body having an interior having an opening to receive an endoscope along the longitudinal axis, the body having a cylindrical portion, the cylindrical portion comprising a first set of protuberances being spaced apart and circumferentially arrayed with respect to one another and extending from the cylindrical portion, the first set of protuberances each comprising a bottom surface having a first raised surface and a second raised surface disposed on a distal end, an inner end and an outer edge to engage tissue, the elongated body further comprising a second set of protuberances being spaced apart and circumferentially arrayed with respect to one another and extending from the elongated body, each of the second set of protuberances having an inner end and an outer edge to engage tissue, and a window disposed between the outer edge and the inner end;

FIG. 41B illustrates a side view of the fitting shown in FIG. 41A;

FIG. 41C illustrates a top view of the fitting shown in FIG. 41A;

FIG. 42A illustrates a perspective view of another embodiment of a fitting wherein the fitting comprises six protuberances;

FIG. 42B illustrates a top view of the fitting shown in FIG. 42A;

FIG. 42C illustrates a side view of the fitting shown in FIG. 42A;

FIG. 42D illustrates a cross-sectional view of the fitting shown in FIG. 42A;

FIG. 43A illustrates a perspective view of another embodiment of a fitting wherein the fitting comprises six protuberances and defines a tab;

FIG. 43B illustrates a top view of the fitting shown in FIG. 43A;

FIG. 43C illustrates a side view of the fitting shown in FIG. 43A; and

FIG. 43D illustrates a cross-sectional view of the fitting shown in FIG. 43A.

Like reference numerals indicate similar parts throughout the figures. It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

In some embodiments, a fitting for a medical scoping device is provided that reduces the risk of complications during a procedure. A medical scoping device fitting is provided, which allows for improved visualization of a biological lumen (e.g., colon, small bowel, etc.). In some embodiments, there is a medical scoping device fitting that expands the folds of the biological lumen to improve visibility of the lining of the biological lumen. In some embodiments, there is a disposable medical scoping device fitting that is compressible to allow access into narrower portions of a biological lumen and debris from the lumen to pass by the fitting.

The fitting comprises a plurality of protuberances that are configured to project outwardly or inwardly relative to the body. Protuberances or projections include, for example, fingers, wings, bristles, spikes, spines, fins, wedges, paddles, cones or the like that have flexibility characteristics to contact and unfold the biological lumen. The protuberances or projections (e.g., fingers, wings, bristles, spikes, spines, fins, wedges, paddles, cones, etc.) place an expansion force against the walls of a biological lumen to straighten the folds present in the wall of the lumen. The flexibility of the protuberances allows the fitting to provide adequate expansion forces to variously sized portions of the biological lumen without damaging tissue. In this way, the methods and devices of the present disclosure are used to increase visualization of the biological lumen (e.g., colon, esophagus, etc.) during a medical scoping procedure (e.g., colonoscopy or endoscopy). The protuberances of the current application are configured to expand (e.g., move outward) from the body of the fitting and unfold to contact the biological lumen (e.g., colon) as the fitting disposed on the medical scoping device is moved proximally in the biological lumen. This is so as the protuberances encounter resistance and friction from the lumen tissue as the fitting is moved proximally in the lumen. The protuberances contact the folds in the lumen and allow the folds to open so that visualization of the lumen is easier.

It will be understood that a fitting includes a cap or covering for a medical scoping device.

The protuberances of the current application are configured to fold, flatten, or move inward relative to the body of the fitting as the medical scoping device is moved distally in the biological lumen.

In some embodiments, the fitting is configured such that the protuberances comprise a gradient moment of flexibility from one end of the protuberance to the other. That is, the protuberances are configured to have a varied degree of flexibility along its length. In some embodiments, the change in flexibility is gradual.

In some embodiments, the protuberances have regions of high flexibility directly adjacent to regions of lower flexibility. In some embodiments, the variability in flexibility is customized or varied by the thickness of the protuberances. The flexibility of the protuberances can be increased or decrease by, among other things, increasing or decreasing the thickness of discrete regions of the protuberance to create one or more pivot points on the protuberance, disposing different notches or recesses at discrete regions of the protuberance, increasing or decreasing the width or surface area at discrete regions of the protuberance, increasing or decreasing windows or cutouts at discrete regions of the protuberance, and/or controlling the elasticity at discrete regions of the protuberance. In some embodiments, controlling contact points and friction with the lumen wall can be accomplished by increasing or decreasing the angles of the protuberances, having different contours of the edges of the protuberances, and/or having different raised surfaces or inclines on or in the protuberances.

In some embodiments, a fitting/cap for an endoscope is provided that includes protuberances/wings that are longer than standard fitting/cap protuberances to increase visibility during a procedure. In some embodiments, a fitting/cap for an endoscope is provided that includes protuberances/wings that are longer than standard fitting/cap protuberances to increase visibility during a procedure. The fitting/cap includes two raised surfaces under the protuberances/wings, such as feet. The two feet are positioned at the end of the protuberances/wings and are rounded off. In some embodiments, a fitting/cap for an endoscope is provided which includes a second row/set of protuberances/fingers that are in-line with a top row/set of protuberances/fingers in order to have clear paths for debris to move through (to avoid pushing debris). The fitting/cap does not have altered protuberance/finger length, but includes raised surfaces such as two feet on the bottom of a first row/set of protuberances/fingers that are rounded. In some embodiments, a fitting/cap with a 70 durometer hardness is provided which is more easily removed from the endoscope. In some embodiments, a fitting/cap with a slim profile is provided.

These embodiments may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “top” and “bottom” are relative and used only in the context to the other, and are not necessarily “upper” and “lower”.

### Fitting

The following discussion includes a description of a fitting for a medical scoping device in accordance with the principles of the present disclosure. Alternate embodiments are also disclosed. Reference is made in detail to the exemplary embodiments of the present disclosure, which are illustrated in the accompanying figures. A fitting includes, for example, a cover, cap or top for the medical scoping device. In some embodiments, the fitting releases and/or is removed from the medical scoping device. In some embodiments, upon removal of the fitting, the fitting can be torn or damaged to prevent reuse. In some embodiments, the fitting allows a user to remove the fitting from the medical scoping device without the fitting being damaged or destructively removed.

The components of device discussed herein can be fabricated from biologically acceptable materials suitable for medical applications, including synthetic polymers. For example, the components of the device, individually or collectively, can be fabricated from materials such as machined or injection molded thermoplastics such as polyaryletherketone (PAEK) including polyetheretherketone (PEEK), polyetherketoneketone (PEKK) and polyetherketone (PEK), carbon-PEEK composites, PEEK-BaS04 polymeric rubbers, polyethylene terephthalate (PET), fabric, silicone, polyurethane, silicone-polyurethane copolymers, polymeric rubbers, polyolefin rubbers, semi-rigid and rigid materials, elastomers, rubbers, thermoplastic elastomers, thermoset elastomers, elastomeric composites, polyphenylene, polychloropene, polyamide, polyetherimide, polyethylene, epoxy, partially resorbable materials, totally resorbable materials, polyglycolide, polytyrosine carbonate, polycaprolactone, silicone based rubber, liquid silicone rubber, High Consistency Rubber, silicon, TPE, Polypropylene, Polycarbonate, ABS or any combination thereof.

The components of the device, individually or collectively, may also be fabricated from a heterogeneous material such as a combination of two or more of the above-described materials. The components of device 10 may be monolithically formed, integrally connected or include fastening elements and/or instruments, as described herein. It is preferred that the devices as described herein are constructed of a suitable biocompatible material to impart various desirable characteristics, such as flexibility, resilience, and deformability.

The components of the device disclosed herein may be coated with a lubricant to facilitate insertion of the fitting into a biological lumen and advancement through said lumen. Suitable lubricants include, but are not limited to, hydrogel polymers such as poly(2-hydroxyethyl methacrylate) (PHEMA) and ComfortCoat®, suitable hydrophobic agents include, but are not limited to, silicone, glycerine, olive oil, castor oil, chlorotrifluoroethylene (CTFE oil) and polyphenyl ethers or a mixture thereof. The lubricant may be sprayed or brushed onto the outer surface of the disclosed devices. In some embodiments, the lubricant is coated only onto the distal end of the device so that only the outer surface of distal end of the fitting is coated with the lubricant.

In some embodiments, the fitting may have a modulus of elasticity in the range of about 1×10² to about 6×10⁵ dyn/cm², or 2×10⁴ to about 5×10⁵ dyn/cm², or 5×10⁴ to about 5×10⁵ dyn/cm² or about 1×10² to about 6×10⁵ dynes/cm², or 2×10⁴ to about 5×10⁵ dynes/cm², or 5×10⁴ to about 5×10⁵ dynes/cm².

As used herein, the term “medical scoping device” refers to any or all of endoscopes, enteroscopes, sigmoidoscopes, gastroscopes, colonoscopes and panendoscopes, or other suitable devices for insertion into a biological lumen and visualization therein. Medical scoping device is used interchangeably and is intended to include all scoping instruments whether passed directly or through a cannula into a body/organ/tissue cavity. Endoscopy involves the inspection of the inside of the body or body cavity and includes arthroscopy, cystoscopy, gastroscopy, uteroscopy and colonoscopy whereas enteroscopy is the examination of the small intestine including the duodenum, jejunum, and ileum. In all instances the scopes are elongate flexible probes and it is intended that the covers of the present invention may be used in conjunction with all of the aforementioned scopes.

In some embodiments, the components of the devices disclosed herein are disposable. Thus, fitting 10 is configured to be discarded following use. Further, the devices set forth herein may be made of a low cost, disposable material so that labor and cost associated with cleaning and autoclaving is avoided.

Accordingly an “endoscopic procedure” is intended to include any medical procedure or examination that involves use of an endoscope as hereinbefore described.

As disclosed herein, the distal end of fitting 10 is commensurate with the distal end of an endoscope shaft which comprises lenses, or channels, such as air suction, conduits, biopsy channels, and light guides. The distal end of the endoscope is furthest from the medical practitioner and as such is the end of the endoscope which is deepest within the patient's biological lumen. Thus, the distal end comes into contact with folds of the lining and looped segments of the biological lumen. Accordingly, distal movement of the endoscope is a forward movement into a patient's bowel. Conversely, the proximal end of the endoscope is the end situated nearest the operator. Thus, proximal movement of the endoscope is a backward movement towards the operator. The endoscope is moved distally during intubation and moved proximally during extubation, where the fitting then contacts the folds of the biological lumen.

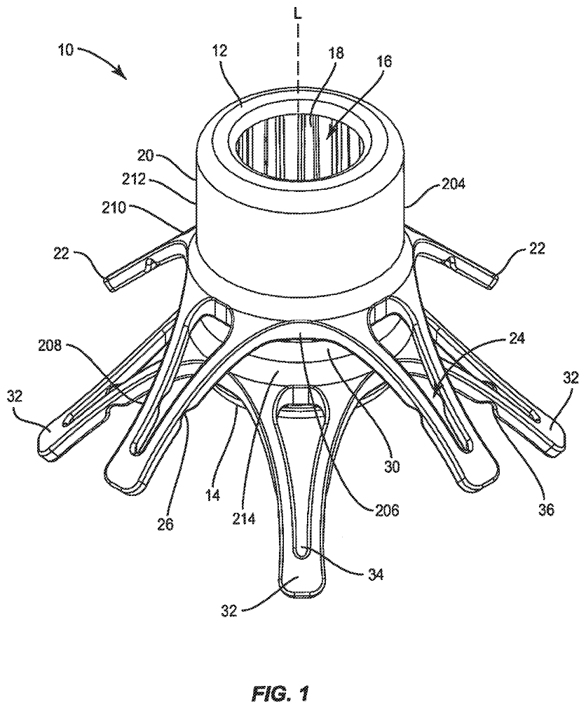

Turning now to FIGS. 1-5, there are illustrated components of a fitting 10 for an endoscope. In some embodiments, fitting 10 is a cap or covering configured to be placed at the distal end of the endoscope. As shown in FIG. 1, fitting 10 extends along longitudinal axis L between a distal end 12 and a proximal end 14. Fitting 10 includes a central channel 16 extending coaxially along the longitudinal axis L. Channel 16 is configured to receive and engage a sidewall of an endoscope. An inner wall of channel 16 includes a plurality of ribs 18 to increase a friction fit between fitting 10 and the sidewall of an endoscope. Ribs 18 extend along the longitudinal axis to prevent unintended rotational movement of fitting 10 relative to the endoscope. In some embodiments, fitting 10 is made from an elastomeric material to facilitate stretching to engage with a variety of endoscopes having varying diameters. In some embodiments, fitting 10 is configured to engage the distal tip of an endoscope. In some embodiments, fitting 10 is configured to engage the distal end of an endoscope adjacent to the distal tip. For example, fitting 10 may be positioned around the distal end of an endoscope, but spaced 1 mm to about 30 mm from the distal tip of the endoscope.

Fitting 10 includes a first region, such as, for example, a first cylindrical member 20 and a second region, such as, for example, a second cylindrical member 30. Cylindrical member 20 includes at least one flexible wing, such as, for example, a protuberance 22. As shown in FIGS. 1-5, cylindrical member 20 includes four protuberances 22. In some embodiments, however, cylindrical member 20 may have more or less than four protuberances 22. For example, cylindrical member 20 may have one, two, three, five, six, seven, eight, nine, ten or more protuberances 22. Protuberances 22 are arranged in a row and extend outward from cylindrical member 20 between an inner end and an outer edge. Protuberances 22 are each evenly spaced apart from one another and radially arranged about an outer surface of cylindrical member 20. As shown in FIG. 1, protuberances 22 include a wide inner end that tapers to an outer edge that is narrower relative to the inner end. This configuration provides stability to protuberances 22 by adding support to the base of the protuberances 22.

Similar to cylindrical member 20, cylindrical member 30 includes at least one flexible wing, such as protuberance 32. As shown in the figures, cylindrical member 30 comprises four protuberances 32. In some embodiments, however, cylindrical member 20 may have more or less than four protuberances 22. For example, cylindrical member 20 may have one, two, three, five, six, seven, eight, nine, ten or more protuberances 22. Similar to protuberances 22, protuberances 32 are arranged in a row and each extend outward from cylindrical member 30 between an inner end and an outer edge. Additionally, protuberances 22 are each evenly spaced apart from one another and radially arranged about an outer surface of cylindrical member 20. Member 20 is oriented relative to member 30 such that protuberances 22 are offset from protuberances 32.

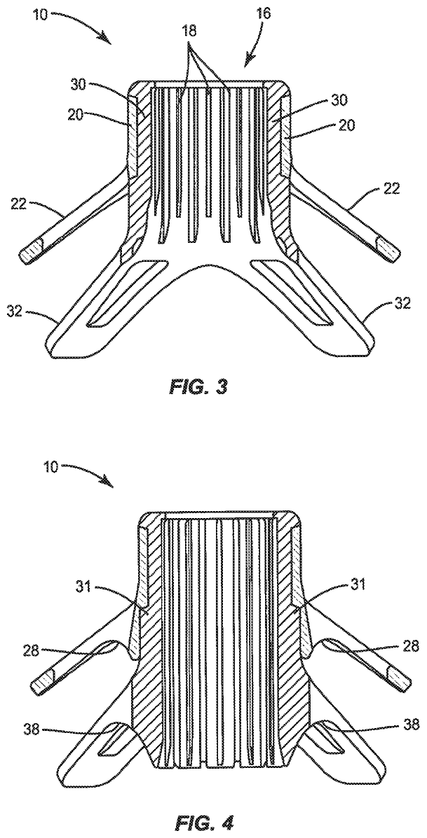

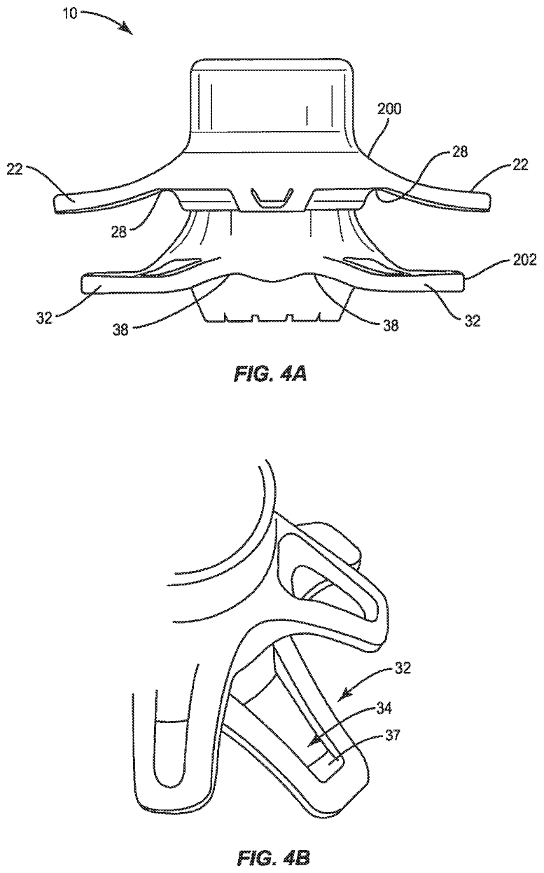

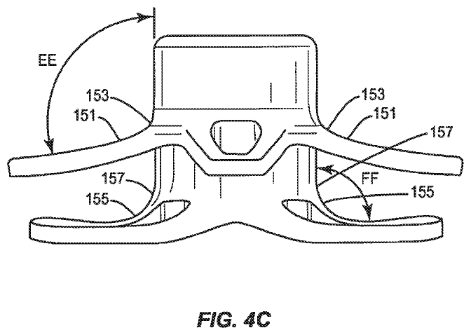

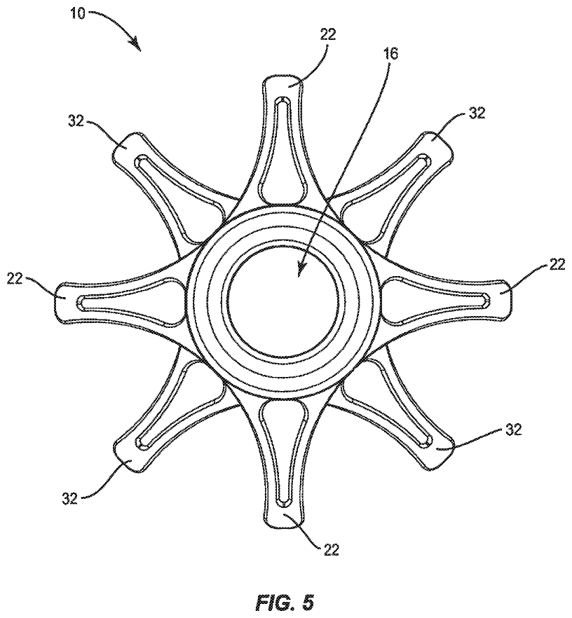

In some embodiments, member 20 is separable from member 30, as shown for example in FIG. 3. Member 30 includes a circumferential recess configured to receive member 20. Member 20 includes an inner diameter complementary to the outer diameter defined by the recess to facilitate a friction fit between members 20, 30. In some embodiments, as shown in FIG. 4, member 30 includes at least one locking member 38 configured to engage a complementary groove in member 20. Locking member 38 is configured to limit or prevent rotation of member 20 relative to member 30. As shown in FIG. 3, member 30 may include a flared proximal end of channel 16 to facilitate insertion of an endoscope. Member 30 also includes a lip at distal end 12 to engage with the tip of an endoscope. Thus, the lip provides a stopping mechanism to ensure that fitting 10 engages the endoscope through the entirety of channel 16, and also ensures that fitting 10 does not slide beyond the distal tip of the endoscope.

In some embodiments, member 20 includes an amount of protuberances that varies from that of member 30. For example, in some embodiments, member 20 may include one less protuberance than member 30 to facilitate insertion and distal advancement of an endoscope into a biological lumen. In various embodiments, the separability of member 20 from member 30 allows for mixing and matching of variously configured protuberances 22, 32 according to the needs of an endoscopic procedure.

In some embodiments, there may be additional circumferential members configured to engage with member 30. For example, member 30 may include an additional circumferential recess to receive an additional cylindrical member having a row of protuberances. Additionally, in some embodiments, members 20, 30 may include more than just a single row of protuberances. For example, members 20, 30 may include two, three, four, five, six, seven, eight, nine or ten rows of protuberances. The rows on members 20, 30 may be arranged such that the protuberances of neighboring rows are staggered with respect to one another. Alternatively, the rows of protuberances may be arranged such that the protuberances of neighboring rows are aligned with one another. Members 20, 30 may be elongated to accommodate multiple rows of protuberances to allow the protuberances a full range of flexible motion without interfering with neighboring protuberances.

As shown in FIGS. 1-5, protuberances 22, 32 include central windows 24, 34 which extend from the inner end toward the outer edge 202 of the protuberances. The protuberances project out from the body 204. Protuberances can be spaced apart from each other by arc 206. Windows 24, 34 allow for added flexibility of protuberances 22, 32 when being advanced or withdrawn through a biological lumen. The windows or cutouts run in a longitudinal axis along the protuberance and also provide a degree of firmness that assists in unfolding the biological lumen folds as the fitting 10 is moved proximally in the lumen. Additionally, the presence of windows 24, 34 allows the protuberances to bend from side to side, which enables a medical practitioner to turn, withdraw or move forward an endoscope within the biological lumen without potentially damaging tissue. Furthermore, in some embodiments, protuberances 22, 32 may include notches, such as, for example recesses or notches 26, 36. Recesses or notches 26, 36 provide an easily flexible point along the length of the protuberances 26, 36 which are configured to flex prior to flexure of the rest of the protuberance. Recesses or notches 26, 36 provide the desired flexibility as the thickness in these areas is reduced to provide the pivot points. In some embodiments, they are disposed in the middle portion 208 of the protuberance and provide pivot points so that the outer edges of the protuberance can be bent from side to side, which enables a medical practitioner to turn, withdraw or move forward an endoscope within the biological lumen without potentially damaging tissue. It will be understood by those of ordinary skill in the art that although one recess or notch is shown on each protuberance, two, three, four, five, six or more recesses or notches can be on one protuberance to have the desired flexibility.

In FIG. 1, there is a lower rim 214 to support the second row of protuberances that surround the body and there is upper rim 210 to support the first row of protuberances. The distal end 12 of the fitting is smooth (there are no recesses or projections) as for ease of insertion into a biological lumen. Likewise, sidewall 212 is also smooth (there are no recesses or projections) as for ease of insertion into a biological lumen. The medical scoping device will be covered or capped by fitting 10. The medical scoping device will have the fitting placed on it along the fittings longitudinal axis shown as L. The fitting will not impair the view of the medical scoping device. Outer edge 202 is shown with a reduced width and surface area as compared to inner end 200. In some embodiments, outer edge 202 can be contoured or be free of sharp edges or points so as to prevent damage to the interior of the biological lumen. In FIG. 1, there are 4 protuberances about the lower rim and 4 protuberances about the upper rim. It will be understood that the fitting can have from about 4 to about 18 protuberances per row or per fitting. For example, there can be from about 4, 5, 6, 7, 8, 9, 10, 11, 12, 13, 14, 15, 16, 17, to about 18 protuberances circumferentially arrayed about the fitting. In some embodiments, the protuberances are spaced apart from each other by from about 0.25 cm to about 2.5 cm.

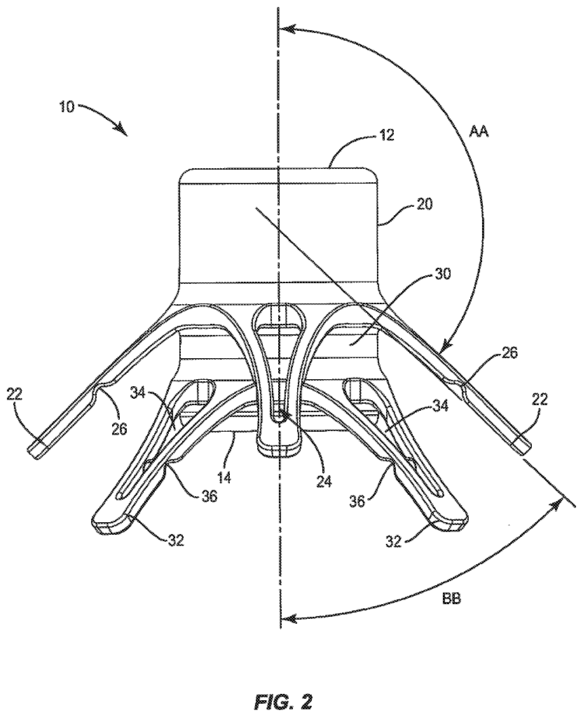

FIG. 2 illustrates a side view of the fitting shown in FIG. 1. The fitting is shown where the protuberances are moved circumferentially inward, where the angle AA from the top of the longitudinal axis of the fitting to the top of the protuberance is from about 100 degrees to about 150 degrees and the angle BB from the bottom of the protuberance to the bottom of the fitting is from about 30 degrees to about 80 degrees.

In some embodiments, the protuberances are positioned at an angle AA from the top of the longitudinal axis of the fitting to the top of the protuberance of from about 100° to 115°, 1200 to 1300, 135° to 1400 or 145° to about 160°. In some embodiments, the protuberances are positioned at an angle BB from the bottom of the protuberance to the bottom of the longitudinal axis of the fitting from about 30°, 35°, 40°, 45°, 50°, 55°, 60°, 65°, 70°, 75°, to about 80°. These angles can vary as the protuberances are at their rest position, their circumferentially inward position, and in their circumferentially outward position.

In some embodiments, as shown in FIGS. 4-4B, protuberances 22, 32 may include variable thicknesses along their lengths to affect the flexibility of the protuberances. For example, as shown in FIGS. 4 and 4A, each protuberance 22 includes a reinforced portion 28, and each protuberance 32 includes a similar reinforced portion 38. The reinforced portion 28, 38 comprises a buttress at the root of the protuberance which increases mechanical stability and reduces flexibility of the inner end 200 of the protuberance. In some embodiments, the reinforced region has a peak von Mises stress or tensile stress of from about 3.2×10³ to about 9.8×10³ psi or 5.2×10³ to about 9.5×10³ psi or 7.2×10³ to about 8.868×10³ psi. The reinforced region 28, 38, in the embodiment shown, can be a region that has increased thickness relative to the middle portion or outer edge of the protuberance.

In some embodiments, as shown in FIG. 4C, protuberances 151, 155 include uniform thicknesses along their bodies along their lengths to allow for greater flexibility at all points along the lengths of the protuberances. Protuberances 151 are disposed with a first region of the fitting and protuberances 155 are disposed in a second region of the fitting. Protuberances 151 include an inner end 153, and protuberances 155 include an inner end 157. Inner ends 153, 157 extend from the exterior body of the fitting and include a thickness that is uniform across the entire length of the protuberances 151, 155. Unlike protuberances 22, 32, protuberances 151, 155 do not include any reinforced portion. Thus, protuberances 151, 155 are configured to be uniformly flexible along the entire length or substantially the entirely length of protuberances 151, 155. In some embodiments, inner ends 153, 157 have a peak von Mises stress or tensile stress of from about 1.0×10³ to about 2.0×10⁴ psi or 1.3×10³ to about 1.8×10⁴ psi or 1.0×10⁴ to about 1.7×10⁴ psi.

In some embodiments, the outer edges 202 of the protuberances also include an increased thickness to prevent the tip from bending. In various embodiments, it is desirable that the outer edge of the protuberances be resistant to flexing to provide a flat grip from which to provide a friction contacting surface with the lining of a biological lumen, as discussed herein. As shown in FIG. 4, when protuberances 22, 32 are moved to a radially outward position or unfolded position, as discussed herein, flexibility is focused at the portion between the inner end and the outer edge. In some embodiments, one or more protuberances include a sloped or inclined portion 37 adjacent window 34, as shown in FIG. 4B. Sloped or inclined portion extends from a bottom surface to a top surface of the protuberance at an angle relative to the surfaces. Sloped portion or inclined portion 37 is configured to allow a greater surface area for gripping the lining of a biological lumen, while also allowing for greater flexibility than at the thicker outer edge 202 of the protuberance. In some embodiments, only protuberances 32 include a sloped portion or inclined portion 37. In some embodiments, both protuberances 22 and protuberances 32 include sloped or inclined portions 37.

In some embodiments, the protuberances are between about 2 to about 20 mm in length from the inner end to the outer tip. In some embodiments, the protuberances can have a length between about 4 to about 18 mm, between about 7 to about 16 or between about 10 to about 15 mm. In various embodiments, each of the protuberances can be from about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, to about 20 mm in length.

In various embodiments, each of the protuberances has a width of from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. The width can vary throughout the fitting and/or along the protuberance to achieve the desired flexibility.

In various embodiments, the fitting, body, protuberances can have a thickness ranging from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. The thickness can vary throughout the fitting, body and/or along the protuberance to achieve the desired flexibility. For example, at the inner edge of the protuberance the thickness can be about 1 mm to about 1.5 mm, then in the middle portion, the thickness can be about 0.5 mm by the recess or notch, then by the outer edge the thickness can be about 0.75 mm to provide the desired flexibility to the protuberance.

In various embodiments, rows of protuberances or individual protuberances may be variously sized. For example, the row of protuberances 32 may be longer than the row of protuberances 22. In some embodiments, protuberances 32 include a length of about 14 mm, and protuberances 22 include a length of about 11 mm. As shown in FIGS. 1-5 the width of the protuberances tapers from the inner end to the outer edge. The taper may be varied according to the needs of a specific endoscopic procedure. In some embodiments, the protuberances have a width of about 5 mm to about 20 mm toward the inner end and a width of about 1 mm to about 10 mm at the outer edge. In some embodiments, protuberances 32 are wider than protuberances 22. For example, in some embodiments, protuberances 32 have a width of about 11 mm at the inner end and a width of about 6 mm at the outer edge.

In some embodiments, the thickness of each of the protuberances 22, 32 is varied along the length of the protuberances. For example, in some embodiments, as shown in FIGS. 1-5, each of the protuberances includes areas of a first degree of flexibility at the inner end and at the outer edge and an area of a second degree of flexibility in a middle portion between the inner end and the outer edge, the second degree of flexibility being greater than the first degree of flexibility. For example, each protuberance may have a thickness between about 2 mm to about 8 mm at the inner end. In some embodiments, each protuberance may have a thickness between about 4 mm to about 5 mm at the inner end. In some embodiments, each protuberance may have a thickness between about 1 mm to about 3 mm at the outer edge. In some embodiments, each protuberance may have a thickness between about 1 mm to about 2 mm at a middle portion between the inner end and the outer edge. In some embodiments, the thicknesses protuberances may be variously configured. For example, row of protuberances 22 may include a greater thickness at the inner end and outer edge than row of protuberances 32. Alternatively, individual protuberances may have varied thicknesses in relation to adjacent protuberances.

The protuberances are arranged in a radial array about the body 204 of fitting 10. As shown in FIGS. 1-5, there are four protuberances in each row, such that each protuberance occupies less than 90° of the radial space around the body. The distance between each is shown as arc 206 and the distance is configured to allow the protuberances to extend and engage the lining of the biological lumen. In some embodiments, the protuberances are spaced apart by a distance between about 0.1 mm to about 10 mm, about 1 mm to about 7 mm, or about 3 mm to about 6 mm. In other embodiments, there may be less space between protuberances to accommodate embodiments which include an amount greater than four protuberances. The protuberances taper such that each outer edge occupies less than 30° of the radial space around the body. In some embodiments, the protuberances widen along their length such that each outer edge occupies about 90° of the radial space around the body.

In various embodiments, protuberances 22, 32 are angled relative to the longitudinal axis L of fitting 10. As shown, for example, in FIGS. 3 and 4, protuberances 22, 32 are angled at an angle of about 45° relative to the longitudinal axis. In some embodiments, for example, in embodiments where protuberance includes a reinforced portion 28, 38, the angle at the base of the protuberance does not change when the protuberances are being flexed by an external force, as discussed herein. For example, in embodiments in which the protuberances do not include a reinforced portion, the angle at the base of the protuberance changes to a degree depending on the extent of force applied to it.

In various embodiments, when the protuberances 22, 32 are in a resting position, they are acutely angled with respect to the body 204 of fitting 10. In some embodiments, the protuberances are positioned at an angle of about 5° to about 85° with respect to longitudinal axis L of fitting 10. In some embodiments, the protuberances are positioned at an angle of from about 35° to 75°, 45° to 70°, 50° to 65° or 55° to 60° from the cover's central longitudinal axis. In some embodiments, the protuberances are positioned at an angle of about 5°, 10°, 15°, 20°, 25°, 30°, 35°, 40°, 45°, 50°, 55°, 60°, 65°, 70°, 75°, 80°, or 85° relative to longitudinal axis L. In some embodiments, protuberances 22 extending from member 20 are sloped at a more acute angle than protuberances 32 extending from member 30.

In some embodiments, as shown in FIGS. 4A and 4C, the protuberances are movable to an extended configuration. As discussed herein, the protuberances are configured to flare outward relative the longitudinal axis L. For example, as shown in FIG. 4C, protuberances 151, 155 are movable to and angle that is greater than the angle of orientation of the protuberances in the rest position. The protuberances 151 are movable to a first angle EE, and the protuberances 155 are movable to a second angle FF. In some embodiments, angles EE, FF are between about 10° to 180°, 45° to 135°, or about 85° to 95°. In some embodiments, the protuberances are positioned at an angle of about 10°, 15°, 20°, 25°, 30°, 35°, 40°, 45°, 50°, 55°, 60°, 65°, 70°, 75°, 80°, 85°, 90°, 95°, 100°, 100°, 105°, 110°, 115°, 120°, 125°, 130°, 135°, 140°, 145°, 150°, 155°, 160°, 165°, 170°, 175°, or 180° relative to longitudinal axis L when in the extended configuration. In some embodiments, angles EE and FF are movable to the same angle when in the extended position. In other embodiments, angles EE and FF are movable to different angles when in the extended position.

In some embodiments, each of the protuberances 22, 32 are formed from a common elastomeric material so that each of the protuberances possesses common physical properties, such as flexibility. In some embodiments, protuberances that comprise a longer length relative to other protuberances are formed from a more flexible elastomeric material than protuberances of a relatively shorter length.

In some embodiments, protuberances 22, 32 extend substantially straight from the body of fitting 10. In some embodiments, one or more protuberances include a contoured outer edge that is free of sharp edges or points so as to prevent damage to the interior of the biological lumen. Further, the contoured edge acts as a catch when fitting 10 is moved proximally in a biological lumen. The contoured edge provides an initial tissue contacting surface such that when friction is created between the protuberance and the lining, the protuberance moves radially outward from the body and expands to a larger diameter so as to unfold the fold in the biological lumen.

In some embodiments, protuberances 22, 32 are biased to a rest position. As shown in FIGS. 1-5, protuberances 22, 32 are angled in the rest position such that the outer edges of the protuberances face toward proximal end 14 of fitting 10. The flexibility of protuberances 22, 32 allows them to bend either in the proximal direction or the distal direction. Protuberances 22, 32 are movable between a first position, in which protuberances 22, 32, are in a compressed configuration or flat configuration radially inward toward the body 204, and a second position, in which protuberances 22, 32 are in an expanded configuration or projecting radially outward from the body 204. In the first position, the protuberances are pressed toward the body of fitting 10 such that the outer edges of the protuberances move inwardly. When in the first position, fitting 10 has a flat or thinner profile to enable an endoscope to easily be advanced through a biological lumen.

In the second position, the protuberances are expanded radially outward from the body of fitting 10 such that the outer edges of the protuberances move distally from the body. When in the second position, fitting 10 has a wider profile to facilitate contact with the lining of the biological lumen and ability to unfold the folds in the biological lumen for viewing. The protuberances exert a frictional force upon the lining of a biological lumen when in the second position so as to gently force apart the contacted folds that are present in the lining.

In some embodiments, the outer edges of each of the protuberances 22, 32 include a tissue contacting surface configured to engage the lining of a biological lumen. Protuberances 22, 32 comprise an elastomeric material which has a rubbery gripping surface to facilitate the engagement with the lining by maximizing friction created between the surface and the lining.

When advanced through the biological lumen, the angle at which protuberances 22, 32 are disposed with fitting 10 enables the endoscope to glide through the biological lumen with little resistance. However, when the endoscope is retracted and moved in a proximal direction, the outer edges of the protuberances 22, 32 contact the lining of the lumen and flare outward to the second position. The outer edges of the protuberances are blunt so as to avoid damage to tissue during the transition from the first position to the second position. Since protuberances 22, 32 are biased to the rest position, when the protuberances are in the second position; they exert an outward force on the lining of the lumen and create a wider area for the endoscope to visualize.

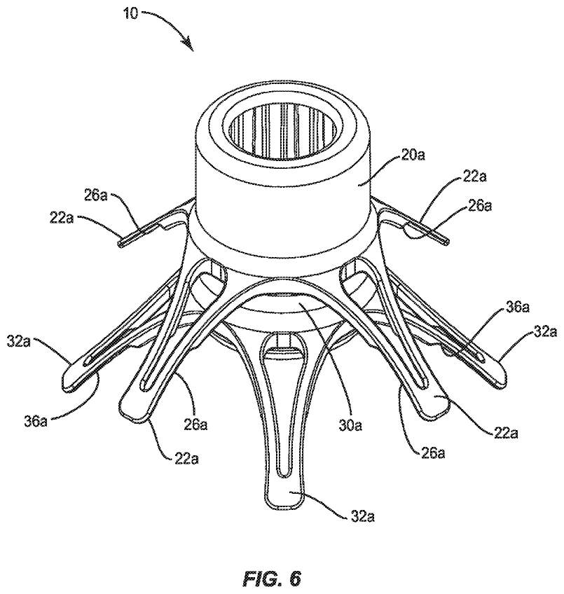

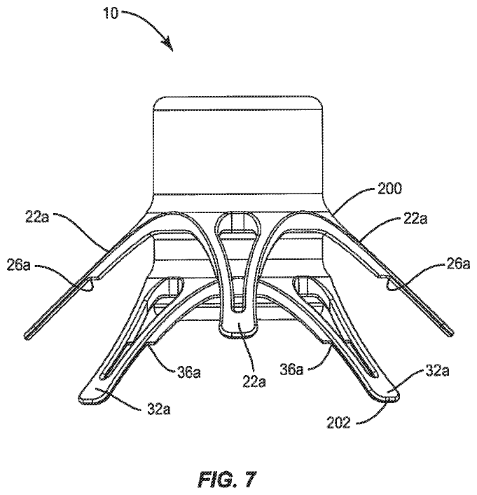

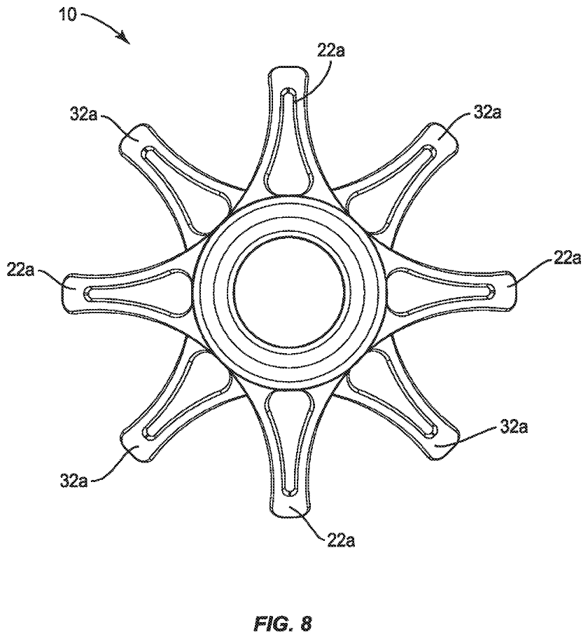

FIGS. 6-8 illustrate a fitting 10 similar to that illustrated in FIGS. 1-5. A first cylindrical member 20a is attached to a second cylindrical member 30a. Cylindrical member 20a includes protuberances 22a, and cylindrical member 30a includes protuberances 32a. Protuberances 22a, 32a are arranged in rows and extend outward from cylindrical members 20a, 30a, respectively, between an inner end and an outer edge. Protuberances 22a, 32a are each evenly spaced apart from one another and radially arranged in rows about the body of fitting 10. The rows of protuberances 22a, 32a may be arranged such that the protuberances of neighboring rows are staggered with respect to one another as shown in FIG. 7. Alternatively, the rows of protuberances may be arranged such that the protuberances of neighboring rows are aligned with one another (not shown).

As shown in FIGS. 6-8, protuberances 22a, 32a include variable thicknesses along their lengths to affect the flexibility of the protuberances. For example, as shown in FIGS. 6 and 7, each protuberance 22a includes a narrowed portion 26a, and each protuberance 32a includes a similar narrowed portion 36a. The narrowed portions 26a, 36a begins at or near the middle portion of the protuberance between the inner ends and outer edges and comprises a region of decreased thickness. The decreased thickness allows for greater flexibility at the outer edges of the protuberance. Thus, the configuration of protuberances 22a, 32a create customized, differing or varied flexibility which allows for a greater degree of flexibility toward the outer end of the protuberances. Therefore, the force required to bend or the bending moment can be increased or decreased as desired. In some embodiments, by increasing the thickness at discrete regions of the protuberance, a reinforced region can be made, such as that shown in the reinforced region 28, 38 of FIGS. 4 and 4A. This region can have increased thickness relative to the middle portion and outer edge of the protuberance. In the embodiments shown in FIGS. 6-8, protuberances have a wider inner end 200 with increased surface area relative to the narrower outer edge 202 of the protuberance.

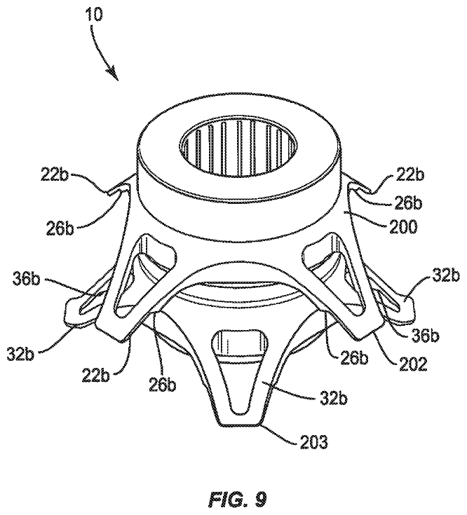

FIGS. 9-11 illustrate a fitting 10 similar to that illustrated in FIGS. 1-5. A first row of protuberances 22b is positioned above a second row of protuberances 32b. As shown in FIGS. 9-11, all protuberances are monolithic. In some embodiments, all protuberances are molded from the same material, and protuberances 22b, 32b are not separable from one another. Protuberances 22b, 32b are arranged in rows and extend outward from the body of fitting 10 between an inner end 200 and an outer edge 202. Protuberances 22b, 32b are each evenly spaced apart from one another and radially arranged in rows about the body of fitting 10. The rows of protuberances 22b, 32b may be arranged such that the protuberances of neighboring rows are staggered with respect to one another (shown). Alternatively, the rows of protuberances may be arranged such that the protuberances of neighboring rows are aligned with one another (not shown). In the embodiments shown in FIGS. 9-11, protuberances have a wider inner end 200 with increased surface area relative to the narrower outer edge 202 of the protuberance.

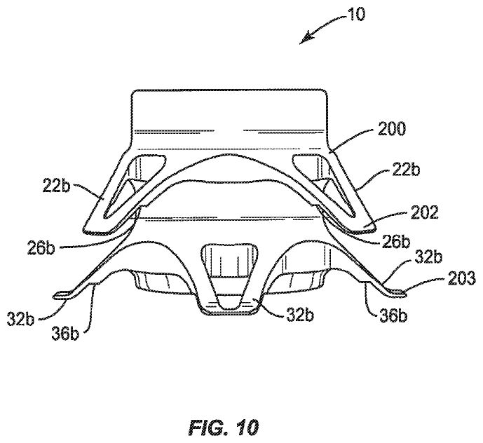

As shown best in FIGS. 9 and 10, protuberances 32b include a flared edge 203 at the outer edge, where there is a widening of the edge. FIGS. 9 and 10, show protuberances at their rest position where there is no external force applied to the protuberance. The flared edge 203 aids in gripping of the lining of a biological lumen during extubation or proximal movement of the endoscope. Specifically, the flared edges act as catches when fitting 10 is moved proximally in a biological lumen, which causes the outer ends of the protuberances to extend outwardly into the second position as disclosed herein. Flared edges are made from an elastomeric material so as to avoid damage to tissue during movement of the endoscope and transition between the first and second positions of the protuberances. Protuberances 22b, 32b include variable thicknesses along their lengths to affect the flexibility of the protuberances, similar to protuberances 22a, 32a. For example, each protuberance 22b includes a narrowed portion 26b, and each protuberance 32b includes a similar narrowed portion 36b. The narrowed portions 26b, 36b begins at or near the midpoint between the inner ends 200 and outer edges and comprises a region of decreased thickness. The decreased thickness allows for greater flexibility at the outer edges 202 of the protuberance. Thus, the configuration of protuberances 22b, 32b create customized, differing or varied flexibility which allows for a greater degree of flexibility toward the outer end of the protuberances.

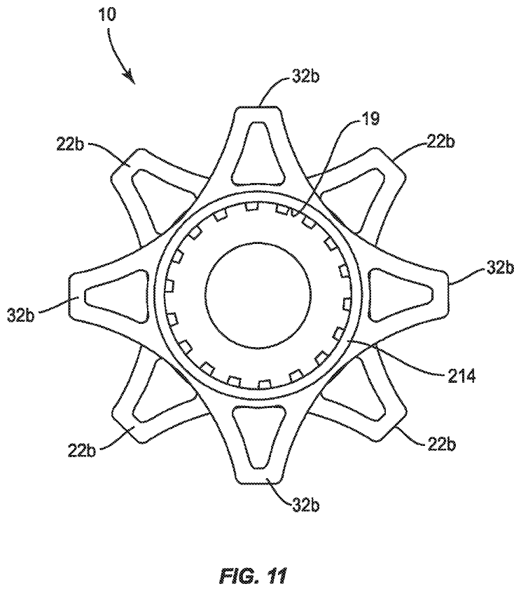

As shown best in FIG. 11, in some embodiments, fitting 10 includes a lip 19 at the distal end of the body. Lip 19 comprises an overmolded portion extending inward over the channel to receive an endoscope. Lip 19 serves as a stop for the distal tip of an endoscope. A medical practitioner inserts the endoscope through the channel 214 until the tip contacts lip 19 to ensure that fitting 10 is secured to the endoscope. The opening in the fitting does not impair the camera view of the endoscope. In some embodiments, the protuberances are spaced from the lip or distal end of the fitting by a distance of from about 1 to about 20 mm, or from about 5 to about 15 mm.

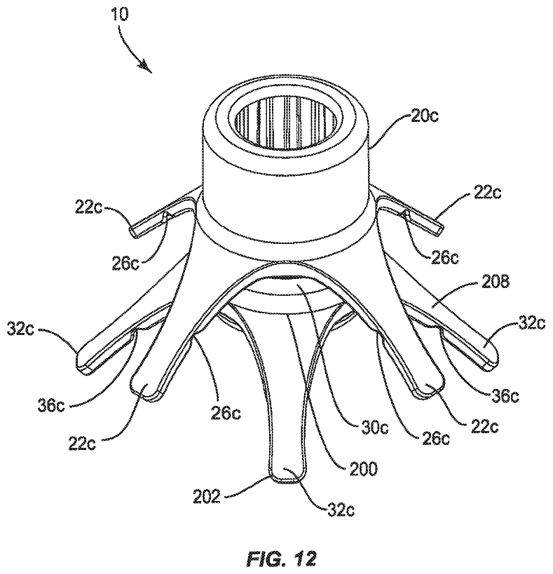

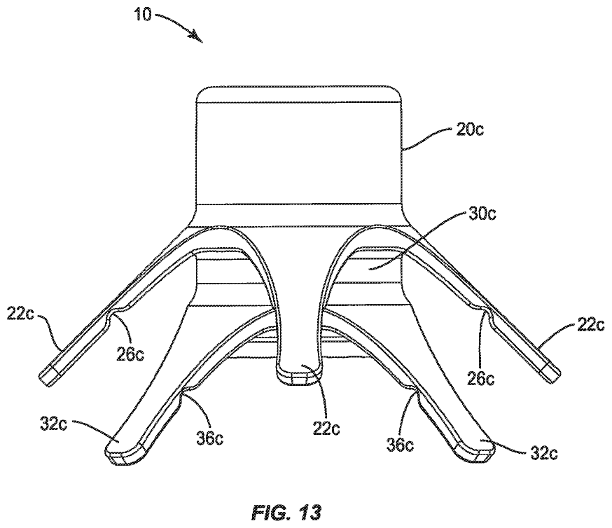

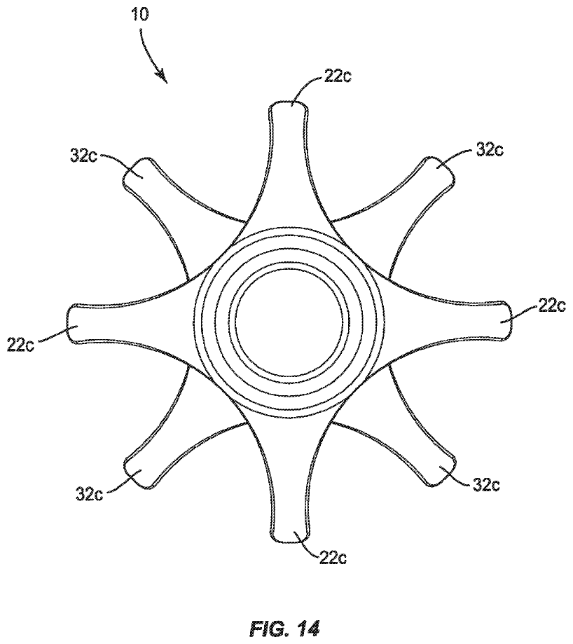

FIGS. 12-14 illustrate a fitting 10 similar to that illustrated in FIGS. 1-5. A first cylindrical member 20c is attached to a second cylindrical member 30c. Cylindrical member 20c includes protuberances 22c, and cylindrical member 30c includes protuberances 32c. Protuberances 22c, 32c are arranged in rows and extend outward from cylindrical members 20c, 30c, respectively, between an inner end 200 and an outer edge 202. Protuberances 22c, 32c are each evenly spaced apart from one another and radially arranged in rows about the body of fitting 10. The rows of protuberances 22c, 32c may be arranged such that the protuberances of neighboring rows are staggered with respect to one another. Alternatively, the rows of protuberances may be arranged such that the protuberances of neighboring rows are aligned with one another.

As shown in FIGS. 12-14, protuberances 22c, 32c are wide at their inner ends 200 and taper to a narrower region at their outer edges 202. Unlike protuberances 22, 32, protuberances 22c, 32c do not include central windows. Protuberances 22c, 32c are configured to be solid to impart properties of greater structural stability. Furthermore, in some embodiments, protuberances 22c, 32c include notches, such as, for example, recesses or notches 26c, 36c. Recesses or notches 26c, 36c provide desired flexibility as the thickness in these areas is reduced to provide the pivot points. In some embodiments, they are disposed in the middle portion 208 of the protuberance and provide pivot points so that the outer edges 202 of the protuberance can be bent from side to side, which enables a medical practitioner to turn, withdraw or move forward an endoscope within the biological lumen without potentially damaging tissue. It will be understood by those of ordinary skill in the art that although one recess or notch is shown on each protuberance, two, three, four, five, six or more recesses or notches can be on one protuberance to have the desired flexibility.

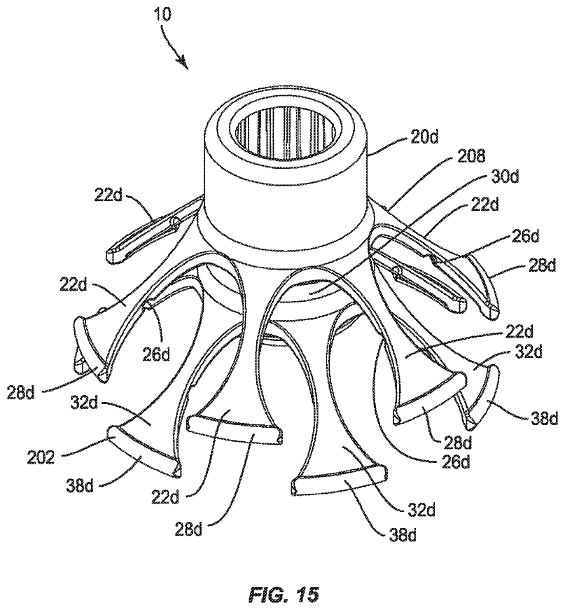

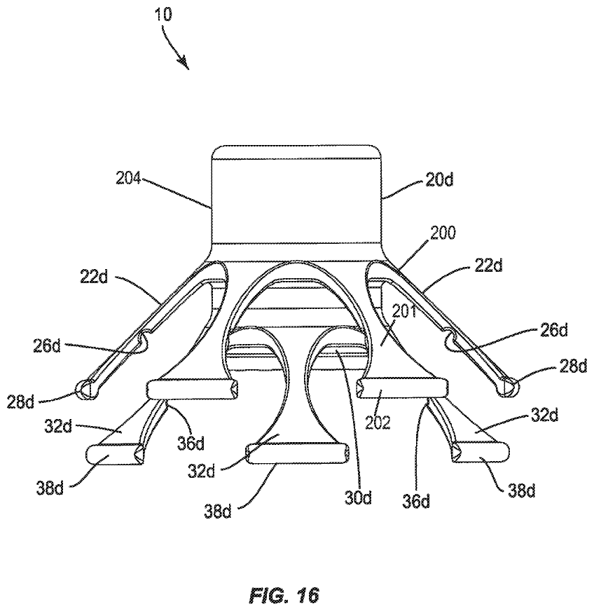

FIGS. 15-17A and B illustrate a fitting 10 similar to that illustrated in FIGS. 1-5. A first cylindrical member 20d is attached to a second cylindrical member 30d. Cylindrical member 20d includes protuberances 22d, and cylindrical member 30d includes protuberances 32d. Protuberances 22d, 32d are arranged in rows and extend outward from cylindrical members 20d, 30d, respectively, between an inner end 200 and an outer edge 202. Protuberances 22d, 32d are each evenly spaced apart from one another and radially arranged in rows about the body of fitting 10. The rows of protuberances 22d, 32d may be arranged such that the protuberances of neighboring rows are staggered with respect to one another. Alternatively, the rows of protuberances may be arranged such that the protuberances of neighboring rows are aligned with one another. Each row of protuberances 22d, 32d includes six protuberances. However, in other embodiments, there may be more or less than six in each row. Inner end 200 of the protuberance extends to middle portion 201 then to outer edge 202. Middle portion 201 of the protuberance is narrow in width and/or surface area relative to inner end 200 and outer edge 202, which will have a larger width and/or surface area. In some embodiments, the protuberance is designed so that the inner end 200 has a larger width and/or surface area and then becomes narrower in width and/or surface area at the middle portion 201 and then width and/or surface area increases at the outer edge 202. One reason for this is that it reduces the amount of force per area on the lining of the biological lumen wall, by increasing the surface area touched by the outer edge of the protuberance. The friction of the fitting against the wall helps to unfold the biological lumen as the medical scoping device is withdrawn rather than just having the protuberances press more firmly against it.

In various embodiments, the inner end 200 of the protuberance will have a larger width than the middle portion 201. The inner end will have a width of from about 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. The width can vary throughout the fitting and/or along the protuberance to achieve the desired flexibility.

In various embodiments, middle portion 201 of the protuberance will have a smaller width than the inner end 200 of the protuberances. The middle portion 201 will have a width of from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm. The width can vary throughout the fitting and/or along the protuberance to achieve the desired flexibility.

In various embodiments, the outer edge 202 of the protuberance will have a larger or smaller width than the middle portion 201. The outer edge 202 will have a width of from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. The width can vary throughout the fitting and/or along the protuberance to achieve the desired flexibility.

In some embodiments, the width of the protuberance, especially nearest the body of the fitting, is narrower than the circumference of the fitting. For instance, there is a 360 degree arc length of the fitting, and the protuberance at the body may be, for example, less than 90 degrees of that arc length. In some embodiments, at the outer edge of the protuberance, it is preferred if the total 360 degrees is covered. One reason for this is that it increases surface area and width to touch the biological lumen wall, but at the body of the fitting there is room for colonic debris to pass as the medical scoping device is inserted.

As shown in FIGS. 15-17A, protuberances 22d, 32d are wide at their inner ends and outer edges, and are narrow in a middle portion between the inner ends and the outer edges. The inner ends and outer edges of protuberances 22d, 32d are wider to impart properties of greater structural stability. Additionally, the width of the outer edges is configured to provide a greater surface area for gripping a lining of a biological lumen. Further, it reduces the amount of force per area on the lining of the biological lumen wall, by increasing the surface area touched by the outer edge of the protuberance. The friction of the fitting against the wall helps to unfold 22d, 32d the biological lumen as the medical scoping device is withdrawn rather than just having the protuberances press more firmly against it.

Protuberances 22d, 32d comprise ridges 28d, 38d at the outer edges. Ridges 28d, 38d are raised surfaces which enhance the engagement of protuberances 22d, 32d with the lining of a biological lumen. Ridges 28d, 38d include a curved surface formed from a material having enhanced gripping properties. Furthermore, in some embodiments, protuberances 22d, 32d include notches, such as, for example recesses or notches 26d, 36d. Recesses or notches 26d, 36d provide an easily flexible point along the length of the protuberances 26d, 36d which are configured to flex prior to flexure of the rest of the protuberance.

Protuberances 22d, 32d taper outward such that each outer edge occupies more than 30° of the radial space around the body. In some embodiments, the protuberances widen along their length such that each outer edge occupies between about 30° and about 60° of the radial space around the body. In some embodiments, the outer edges contact adjacent outer edges such that the outer edges encompass 360° of the radial space around the body 204.

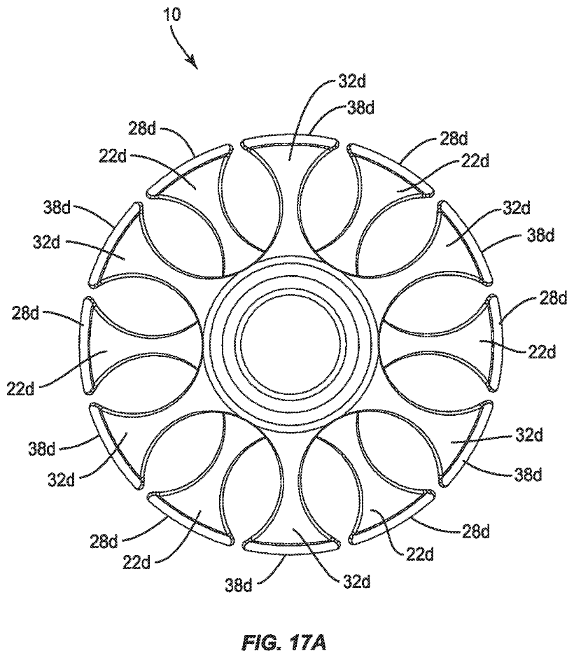

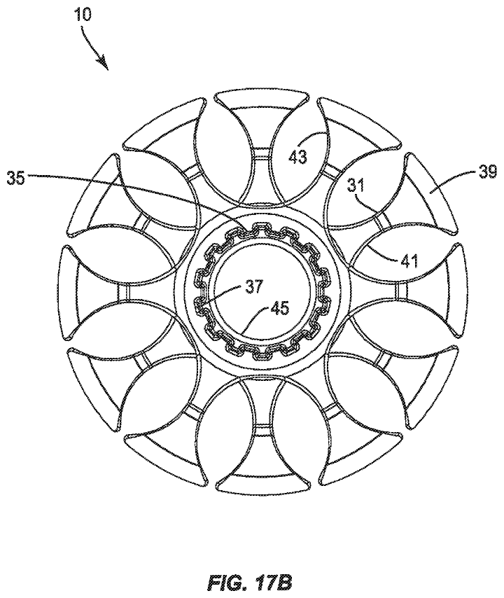

FIG. 17A illustrates a top view of the fitting shown in FIG. 15. FIG. 17B illustrates a bottom view of the fitting shown in FIG. 15, the notches or recesses 31 shown in this view in the middle region of the protuberance increase flexibility of the outer edges of the protuberances and the protuberances 22d, 32d span slightly less than 360 degrees around the body 35 of the fitting. This may allow colonic debris to pass by the fitting in use.

The outer edges 39 of the protuberances have increased width and/or surface area compared to the middle portion of the protuberance and the inner end 41 of the protuberance that will increase contact with the biological lumen lining. To reduce damage of the lining of the biological lumen, the outer edges 39 of the protuberances are contoured. Opening 45 is configured to receive the distal end of the medical scoping device. The body 35 of the fitting, in some embodiments, may have ribs 37 to allow a snug fit with the distal end of the medical scoping device. In some embodiments, the opening 45 may have a diameter that is the same or slightly larger than the diameter of the medical scoping device so that when the fitting is placed on the distal end of the medical scoping device it fits snugly over it. It will be understood that the fitting can have from about 4 to about 18 protuberances per row or per fitting. For example, there can be from about 4, 5, 6, 7, 8, 9, 10, 11, 12, 13, 14, 15, 16, 17, to about 18 protuberances circumferentially arrayed about the fitting. Shown in FIG. 17A, there are 6 protuberances per row.

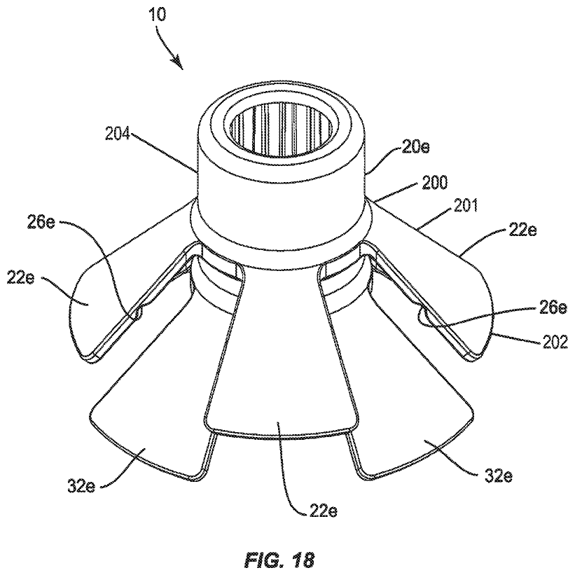

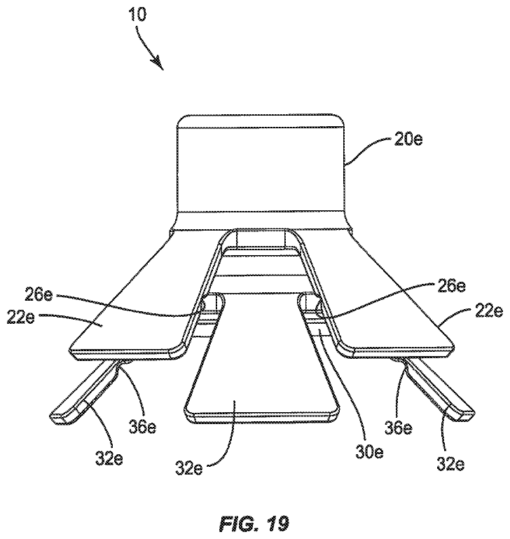

FIGS. 18-20 illustrate a fitting 10 similar to that illustrated in FIGS. 1-5. A first cylindrical member 20e is attached to a second cylindrical member 30e. Cylindrical member 20e includes protuberances 22e, and cylindrical member 30e includes protuberances 32e. Protuberances 22e, 32e are arranged in rows and extend outward from cylindrical members 20e, 30e, respectively, between an inner end and an outer edge. Protuberances 22e, 32e are each evenly spaced apart from one another and radially arranged in rows about the body of fitting 10. The rows of protuberances 22e, 32e may be arranged such that the protuberances of neighboring rows are staggered with respect to one another. Alternatively, the rows of protuberances may be arranged such that the protuberances of neighboring rows are aligned with one another.

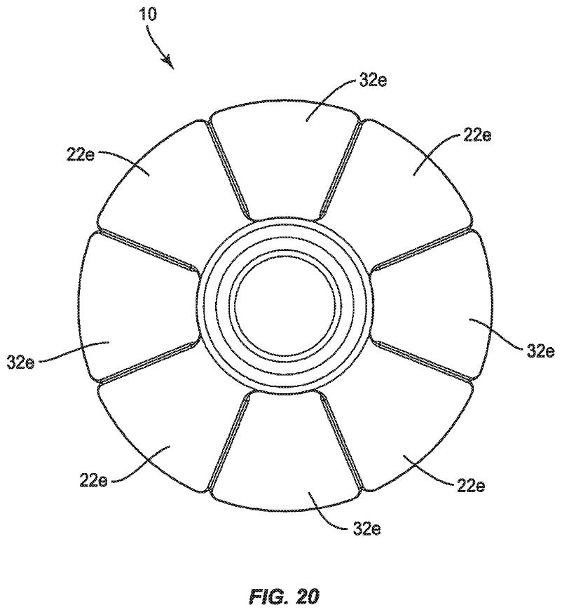

As shown in FIGS. 18-20, protuberances 22e, 32e are wider at its outer edge 202 and taper to a narrower configuration in their middle portion 201 until the narrowest portion at the inner end 200. Inner end 200 of the protuberance extends to middle portion 201 then to outer edge 202. Inner end 200 of the protuberance is narrow in width and/or surface area relative to middle portion 201 and outer edge 202, which will have a larger width and/or surface area. In some embodiments, the protuberance is designed so that the inner end 200 has a smaller width and/or surface area and then becomes wider in width and/or surface area at the middle portion 201 and then the width and/or surface area increases at the outer edge 202. In some embodiments, width of the protuberance, especially nearest the body 204 of the fitting, is narrower than the circumference of the fitting. For instance, there is a 360 degree arc length of the fitting, and the protuberance at the body should be, for example, less than 90 degrees of that arc length. In some embodiments, the outer edge of the protuberance is preferably flared and 360 degrees of the arc length is covered. One reason for this is that it increases surface area and width to touch the biological lumen wall, but at the body 204 of the fitting there is room for colonic debris to pass as the medical scoping device is inserted. In the embodiment shown in FIG. 20, the protuberances are unfolded and radially arrayed 360 degrees about the body of the fitting.

Unlike protuberances 22, 32, protuberances 22e, 32e do not include central windows. Protuberances 22e, 32e have a shape which linearly widens from the inner end to the outer edge and are configured to be solid to impart properties of greater structural stability. Furthermore, in some embodiments, protuberances 22e, 32e include notches, such as, for example recesses or notches 26e, 36e. Recesses or notches 26e, 36e provide an easily flexible point along the length of the protuberances 26e, 36e which are configured to flex prior to flexure of the rest of the protuberance. The protuberances are angled in the embodiment shown in a downward direction relative to the body. In some embodiments, the protuberances are positioned at an angle of from about 5°, 10°, 15°, 20°, 25°, 30°, 35°, 40°, 45°, 50°, 55°, 60°, 65°, 70°, 75°, 800, or 85° in a downward direction relative to the body 204, when the fitting is at a resting position (e.g., there is no external force applied to it by the lining of the biological lumen).

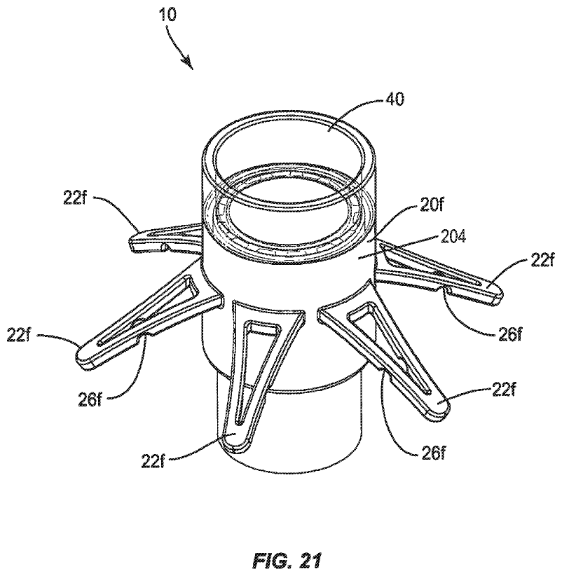

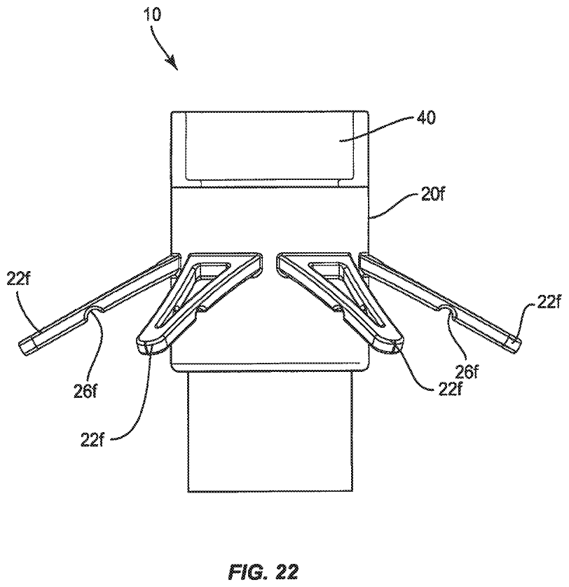

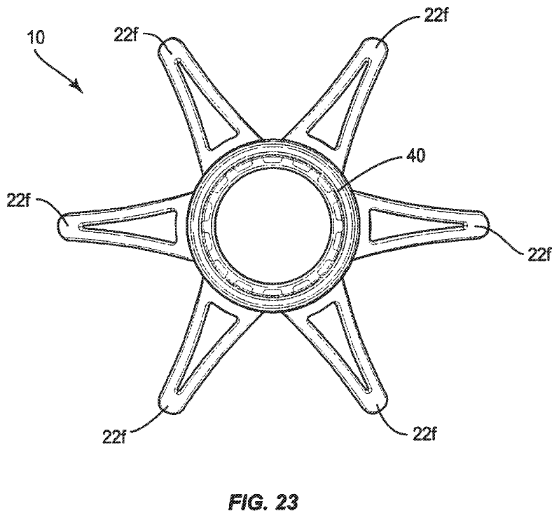

FIGS. 21-23 illustrate a fitting 10 similar to that illustrated in FIGS. 1-5. A cylindrical body 20f includes protuberances 22f, which are arranged in a row and extend outward from cylindrical body 20f between an inner end and an outer edge. Protuberances 22f are evenly spaced apart from one another and radially arranged in a row about the body of fitting 10. In some embodiments, fitting 10 also comprises a cylindrical extender 40 at the distal end of fitting 10. Cylindrical extender 40 is configured to provide a means to spread tissue as an endoscope is advanced through a biological lumen. In some embodiments, cylindrical extender 40 is transparent or semi-transparent to allow visualization through its surface.

The cylindrical extender 40 can be monolithic with the remainder of the fitting (e.g., one piece), alternatively the cylindrical extender can have a diameter that allows it to fit over the fitting which has the medical scoping device head in it. Alternatively, the fitting can be over the distal end of the endoscope first then the cylindrical extender can abut or contact the fitting, such that there are two separate pieces (e.g., the fitting and the cylindrical extender).

The fitting can be soft so as to not damage the lining of the biological lumen. The fitting can be used with suction so as to allow the lining of the biological lumen to collapse around the fitting to further increase visualization and then other elements can be used in conjunction with the fitting (e.g., surgical cutting instruments to remove tissue (e.g., mucosal lining resection), or cauterize tissue, biopsy tissue, etc.).

Protuberances 22f may have an inner end and an outer edge and a recess of notch 26f of reduced thickness disposed in the middle portion of the protuberance to enhance flexibility.

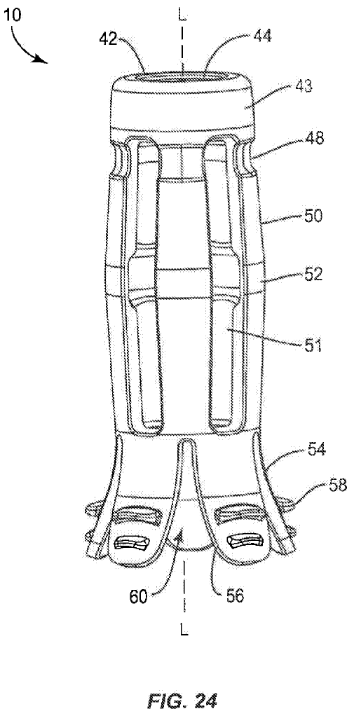

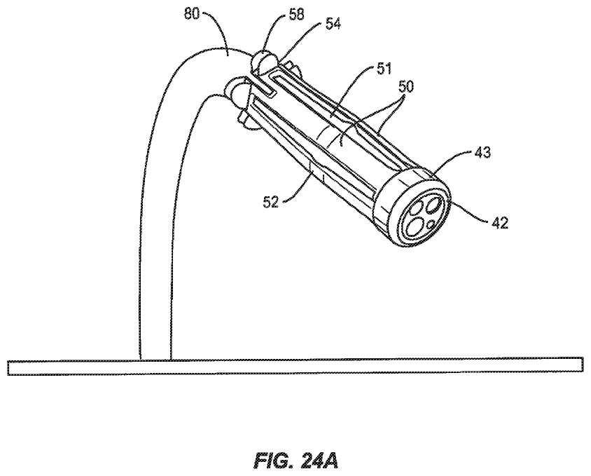

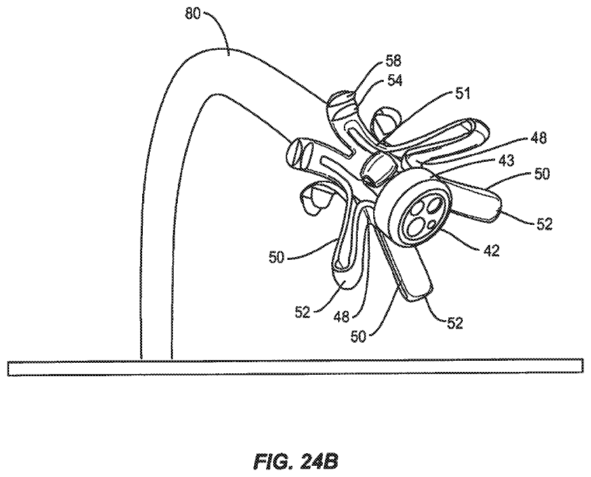

FIG. 24 illustrates a side view of an embodiment of a medical scoping device fitting 10 in accordance with the principles of the present disclosure. FIG. 24 illustrates a side view of the fitting 10, which has a longitudinal axis L. Top 44 of fitting 10 is configured to engage the distal end of an endoscope, as shown in FIGS. 24A and 24B. In some embodiments, top 44 includes a lip on an inner surface configured to serve as a stop for the distal tip of an endoscope. A medical practitioner inserts the endoscope through channel 60 of fitting 10 until the tip contacts the lip to ensure that fitting 10 is secured to the endoscope. Channel 60 extends through the entire length of or substantially the entire length of fitting 10, and is configured to engage and receive the distal end of the medical scoping device. In some embodiments, the body of the fitting can expand around the medical scoping device to provide a snug fit around it so that it does not detach from the distal end of the medical scoping device.

In some embodiments, leading end 42 of top 44 is soft and smooth to facilitate entry into a biological lumen. Fitting 10 further includes a side wall 43 extending between proximal and distal ends of the fitting. In some embodiments, side wall 43 has a smooth finish. The body of fitting 10 includes a plurality of compressible members 52 configured to move between a low profile configuration and an expanded configuration. In some embodiments, fitting 10 includes five compressible members 52 separated by recesses 51. However, in other embodiments, fitting 10 may include more or less compressible members 52. For example, fitting 10 may include one, two, three, four, six, seven, eight, nine or ten compressible members 52.

Compressible members 52 extend along longitudinal axis L of fitting 10. Members 52 include a middle section 50 having an interior cutout, as shown in FIG. 24. The cutout of section 50 defines a lesser thickness than surrounding portions of members 52 to provide a zone of increased flexibility. Thus, upon moving from the low profile configuration to the expanded configuration, members 52 have a tendency to fold at middle sections 50. In some embodiments, compressible members 52 comprise a second cutout 48 at a proximal end adjacent side wall 43. Cutout 48 biases members 52 to fold toward the proximal direction when in the expanded configuration.

The lower end of the body comprises protuberances 54 configured to engage with the walls of a biological lumen. The surface of each protuberance has one or more raised surfaces 58 extending transversely from protuberance 54. It will be understood that the protuberance 54 may have one, two, three, four, five, six or more raised surfaces on the protuberance. Raised surfaces 58 aid in the frictional contact with the lining of the biological lumen. When raised surfaces 58 engage tissue and the endoscope is retraced distally, a force is applied to the raised surfaces to cause a change in shape of the fitting. Each protuberance 54 has a contoured surface 56, which reduces damage to the lumen lining when protuberance 54 comes in contact with it.

FIGS. 24A and 24B illustrate the movement of fitting 10 between the low profile configuration and the expanded configuration. As shown in FIG. 24A, when in the low profile configuration, members 52 are stretched so as to lie flatly against the surface of an endoscope 50. When in this configuration, the endoscope and fitting can easily slide through a biological lumen. As shown in FIG. 24B, when in the expanded configuration, members 52 flare outward to create a greater circumference of fitting 10. In some embodiments, middle section 50 and cutout 48 include areas of lesser thickness to create increased flexibility. When in the expanded configuration, members 52 bend at middle section 50 and flex in a direction toward cutout 48. In other embodiments, members 52 include a uniform thickness and flexibility along their entire length. In FIG. 24B, the raised surfaces 58 are compacted or compressed closer to leading end 42. In this way, the protuberance 54 can cause friction against the lumen lining and unfold the lining of the biological lumen so that it can be viewed. The length of the fitting, in this embodiment, is reduced in the compacted or compressed configuration and therefore, the fitting changes its shape or configuration as shown in FIG. 24B.

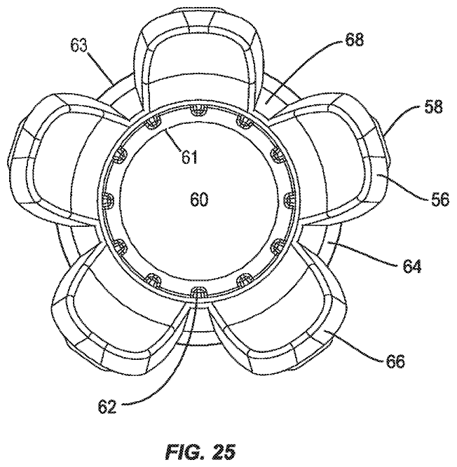

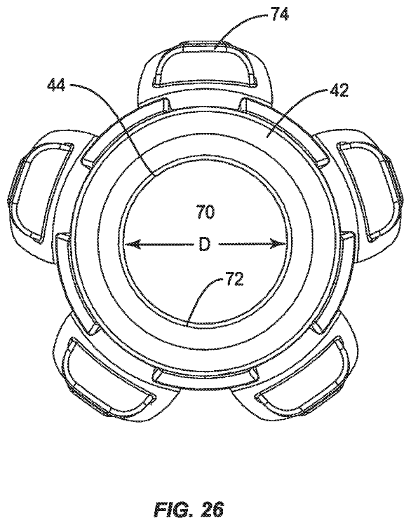

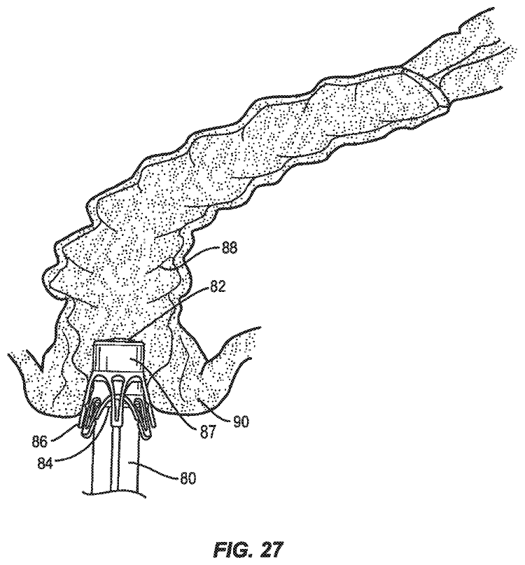

FIG. 25 illustrates a bottom view of the fitting 10 shown in FIG. 24. The bottom of the fitting has opening 60, which is configured to receive the endoscope. The fitting has circular member 64 and rib 62 that are configured to provide support and a snug fit around the distal end of the endoscope. Each protuberance is spaced apart by a distance in arc 68 and the protuberance can have a contoured border 66 that also prevents damage to the lining of the biological lumen. Interior 61 is configured to receive the medical scoping device. Exterior 63 has the protuberances disposed on it.

FIG. 26 illustrates a top view of the fitting shown in FIG. 24. Inner end 74 of the protuberance is shown when it is flat with the body of the fitting. The top 44 of the fitting has opening 70, which is configured to receive the distal end of the endoscope. The diameter of the fitting D has a diameter that is slightly larger than the distal end of the endoscope so that it can cover it and not obstruct the view of the camera or working filed in the endoscope. Leading end 42 of the top of the fitting is smooth for ease of insertion. Circular element 72 around the fitting provides support and allows the fitting to fit snugly on the endoscope.

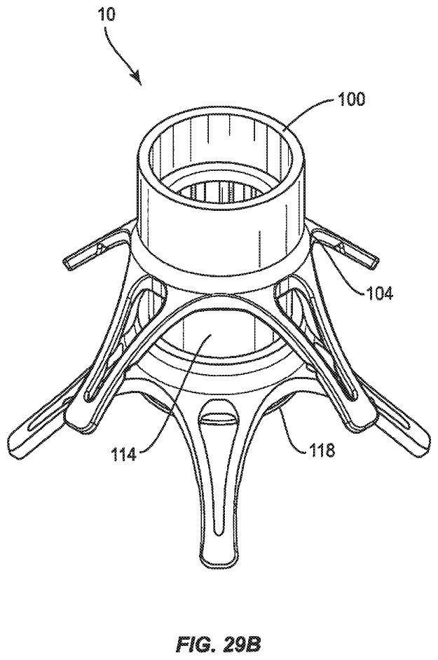

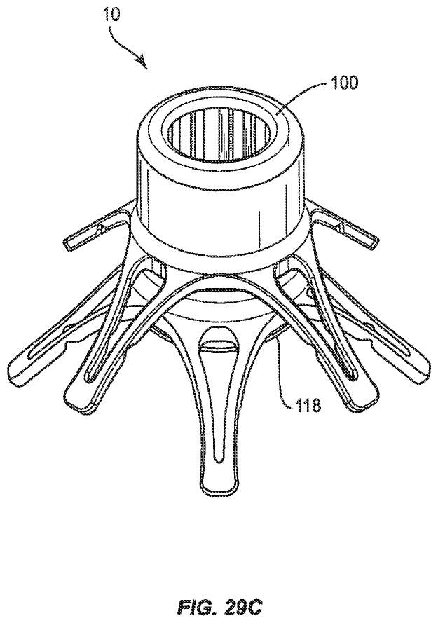

FIG. 29A illustrates a perspective view of an embodiment of a medical scoping device fitting 10 in accordance with the principles of the present disclosure. The fitting illustrated is a two component system, having an upper member 100 and a lower member 114 where two rows of protuberances are present and the fitting can be simply assembled. The upper member 100 can be more flexible than lower member 114. The diameter D2 of the upper member 100 is larger than the diameter D3 of the lower member so that the lower member can engage upper member through opening 102 so that lower member can fit snugly within the upper member 100. Upper member 100 comprises a row of protuberances arranged radially around the upper member. Each protuberance comprises window 110 for the desired flexibility and support of the fitting. Each protuberance can have inner edge 104, which is wider in this embodiment than outer edge 106 of the protuberance. In the middle portion of the protuberance there is a recess or notch 108 to allow the desired flexibility of the protuberance. Each protuberance is spaced apart from each other and has a ridge 112 that engages and can lock into position when contacting ridge 116 of the lower member 114.

Lower member 114 of the fitting 10 can be more rigid and less flexible than the upper member 100. The lower member 104 comprises a row of protuberances arranged radially around the lower member. Each protuberance comprises window 120 for the desired flexibility and support of the fitting. Each protuberance can have ridge 116 that contacts ridge 112 when the lower member 104 is slid into opening 102 of the upper member 100. Ridge 118 of the lower member can provide structural support to the lower member 114. In the middle portion of the protuberance there is a recess or notch 122 to allow the desired flexibility of the protuberance. The distal end of the medical scoping device will be put in opening 103 and 102 once the upper member and lower members are assembled and aligned. The lower member includes ribs 124 which engage the endoscope.

FIG. 29B illustrates a perspective view of a partially assembled medical scoping device fitting shown in 29A, where the upper member 100 slides over the lower member 114. FIG. 29C illustrates a perspective view of an assembled medical scoping device fitting shown in 29A, where the upper member 100 is on lower member and upper member 100 stops at ridge 118 of lower member to prevent upper member from being positioned past ridge 118. The fit between upper and lower member is snug and the lower member 114 would need to be deformed to detach upper member 100 from lower member 114.

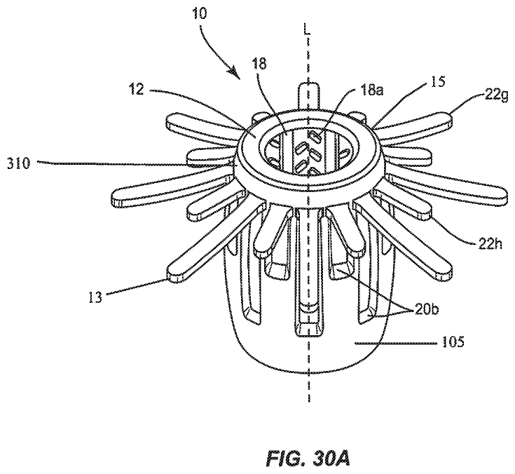

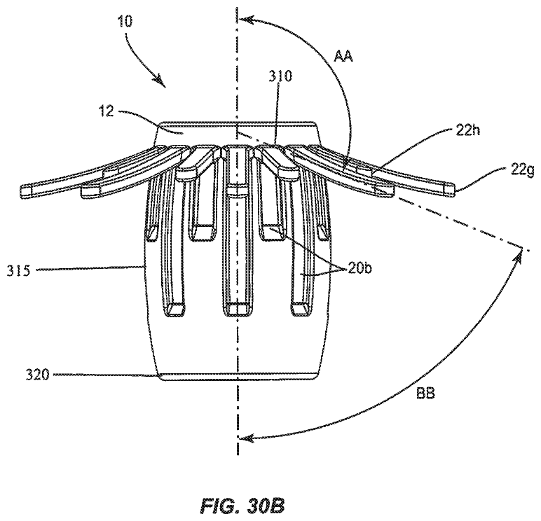

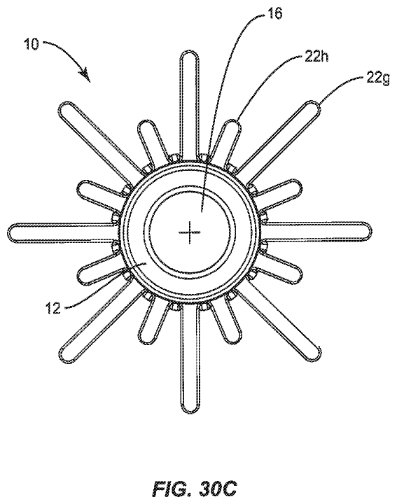

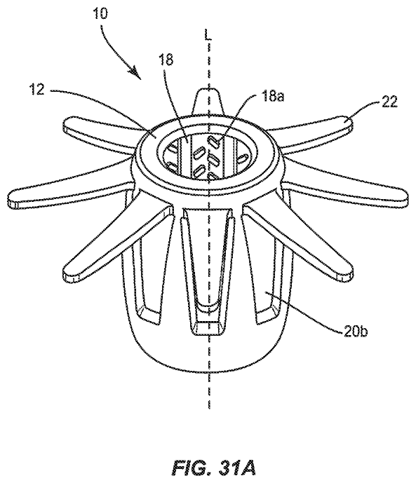

Referring to FIGS. 30A-30C, FIG. 30A illustrates a perspective view of an embodiment of a medical scoping device fitting 10 in accordance with the principles of the present disclosure. The fitting illustrated comprises a single cylindrical member 105 having a longitudinal axis L and a row of protuberances with dual protuberance lengths. Long protuberance 22g and short protuberance 22h spread and stagger evenly near the distal end 12 of the cylindrical member. Each protuberance comprises an outer edge 13 that engages the inner lining of the lumen of the colon and inner end 15. The inner end 15 and the outer edge 13, each have varying flexibility, with the inner end 15 being stiffer and less flexible, in some embodiments, than the outer edge 13, which typically is more flexible and less stiff. The short and long protuberance design pulls the inner lumen of colon tissue closer to the fitting 10 so that it can be better viewed by the medical scoping device. Shorter protuberance 22h stabilizes the fitting and provides greater security in unfolding the inner lumen tissue of the colon. It will be understood that there is one row of protuberances shown. However, there may be two, three, four, five, six, seven or more rows of protuberances. In some embodiments, at least two of the protuberances have a different length. For example, long protuberance 22g and short protuberance 22h are different lengths, but have the same depth and width. The protuberances are arranged in alternating conformations where there is alternating conformation of long protuberance 22g and short protuberance 22h. Shown are the protuberances evenly spaced from one another.

The cylindrical member 105 includes multiple cavities 20b on its exterior. These cavities, in some embodiments, correspond to the shape of the short or long protuberance so that as the protuberance is compressed along the body in a downward direction, the protuberance can engage cavities 20b to further stabilize the fitting 10. In some embodiments, the protuberance can contact the cavity.

In some embodiments, the cylindrical member 105 has a thicker region where the thicker region is at the distal portion compared to the middle portion.

In some embodiments, the interior of the cylindrical member 105 comprises a plurality of raised surfaces 18a and ribs 18 running longitudinally in its interior. The raised surfaces 18a can be angled relative to each other, the longitudinal axis or the rib 18 in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The angles of the raised surfaces 18a do not vary as the device is moved. This is unlike the angle of the raised surfaces of the protuberances as the fitting is moved, which change as the protuberances contact the lining of the colon. The raised surfaces 18a provide contact points with the head of the endoscope and allow the fitting 10 to conform to the surface of the endoscope and provide a friction or snug fit. The ribs 18 can run longitudinally in the interior of the cylindrical member and provide further support for the fitting 10. The ribs 18 and the raised surfaces 18a can be arranged in a pattern between alternating raised surfaces and ribs longitudinally arrayed in the interior of the fitting 10.

FIG. 30B illustrates a side view of an embodiment of a medical scoping device fitting shown in 30A. The protuberances are angled in the embodiment shown in a downward direction relative to the body, where the angle AA from the top of the longitudinal axis of the fitting to the top of the protuberance is from about 95 degrees to about 175 degrees and the angle BB from the bottom of the protuberance to the bottom of the fitting is from about 5 degrees to about 85 degrees.

In some embodiments, the protuberances are positioned at an angle of from about 5°, 10°, 15°, 20°, 25°, 30°, 35°, 40°, 45°, 50°, 55°, 60°, 65°, 70°, 75°, 80°, or 85° in a downward direction relative to the cylindrical member, when the fitting is at a resting position (e.g., there is no external force applied to it by the lining of the biological lumen). These angles can vary as the protuberances are at their rest position, their circumferentially inward position, and in their circumferentially outward position.

The cylindrical member can have proximal region 320, distal region 310 and middle region 315. As shown, the middle region 315 can be wider than the proximal region 320 or the distal region 310 depending on the fit with the medical device. It will be understood that the middle region 315, the proximal region 320 and the distal region 310 can be the same thickness and diameter. Alternatively, the middle region 315 can be smaller than the proximal region 320 or the distal region 310. Alternatively, the middle region 315 and the distal region 310 can be smaller than the proximal region 320. Alternatively, the middle region 315 and the proximal region 320 can be smaller than the distal region 310 depending on the fit with the medical device. In some embodiments, a thicker region can be at the distal region compared to the middle region or a thicker portion can be at the middle portion compared to the distal portion and the proximal portion or a thicker region can be at the proximal portion compared to the middle portion and distal portion.

FIG. 30C illustrates a top view of an embodiment of a medical scoping device fitting shown in 30A. Channel 16 of fitting 10 is configured to receive an endoscope; typically, the channel engages the head of the endoscope and is open so as not to interfere with viewing of the lumen lining.

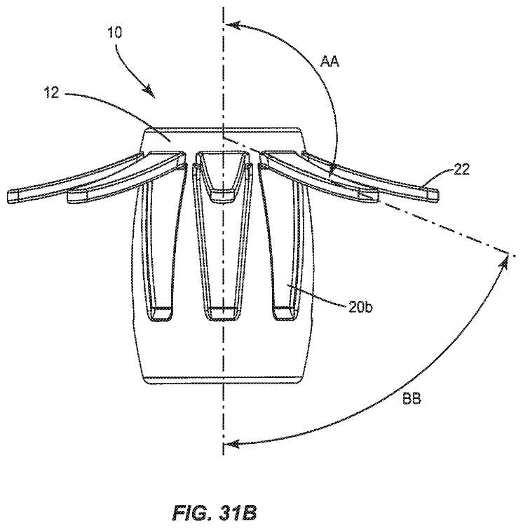

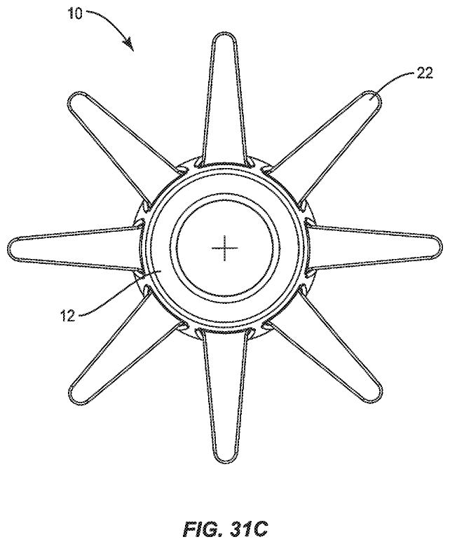

Referring to FIGS. 31A-31C, FIG. 31A illustrates a perspective view of an embodiment of a medical scoping device fitting 10 in accordance with the principles of the present disclosure. The fitting illustrated comprises a single cylindrical member with a row of protuberances 22 spreading evenly near the distal end 12 of the cylindrical member. The cylindrical member includes multiple cavities 20b on exterior walls and inner walls include ribs 18 and a pattern of raised surfaces 18a between two ribs. The protuberances of the fitting can, in some embodiments, have a length, width and depth that is the same as shown in FIG. 31A.

The inner end and the outer edge of each protuberance can have varying flexibility, with the inner end being stiffer and less flexible, in some embodiments, than the outer edge, which typically is more flexible and less stiff. It will be understood that there is one row of protuberances shown. However, there may be two, three, four, five, six, seven or more rows of protuberances. Shown are the protuberances evenly spaced from one another.

The cylindrical member includes multiple cavities 20b on its exterior. These cavities, in some embodiments, correspond to the shape of the protuberance so that as the protuberance is compressed along the body in a downward direction, the protuberance can engage cavities 20b to further stabilize the fitting 10. In some embodiments, the protuberance can contact the cavity.

In some embodiments, the interior of the cylindrical member comprises a plurality of raised surfaces 18a and ribs 18 running longitudinally in its interior. The raised surfaces 18a can be angled relative to each other, the longitudinal axis or the rib 18 in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The angles of the raised surfaces 18a do not vary as the device is moved. This is unlike the angle of the raised surfaces on the protuberances as the fitting is moved, which change as the protuberances contact the lining of the colon. The raised surfaces 18a provide contact points with the head of the endoscope and allow the fitting 10 to conform to the surface of the endoscope and provide a friction or snug fit. The ribs 18 can run longitudinally in the interior of the cylindrical member and provide further support for the fitting 10. The ribs 18 and the raised surfaces 18a can be arranged in a pattern between alternating raised surfaces and ribs longitudinally arrayed in the interior of the fitting 10.

FIG. 31B illustrates a side view of an embodiment of a medical scoping device fitting shown in 31A. The protuberances are angled in the embodiment shown in a downward direction relative to the body, where the angle AA from the top of the longitudinal axis of the fitting to the top of the protuberance is from about 95 degrees to about 175 degrees and the angle BB from the bottom of the protuberance to the bottom of the fitting is from about 5 degrees to about 85 degrees.

In some embodiments, the protuberances are positioned at an angle of from about 5°, 10°, 15°, 20°, 25°, 30°, 35°, 40°, 45°, 50°, 55°, 60°, 65°, 70°, 75°, 80°, or 85° in a downward direction relative to the body, when the fitting is at a resting position (e.g., there is no external force applied to it by the lining of the biological lumen). These angles can vary as the protuberances are at their rest position, their circumferentially inward position, and in their circumferentially outward position.

FIG. 31C illustrates a top view of a medical scoping device fitting shown in 31A with a row of protuberances 22 spreading evenly near the distal end 12 of the cylindrical member 20.

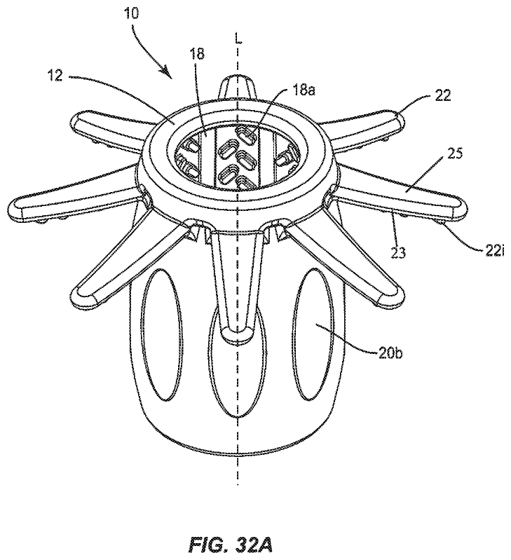

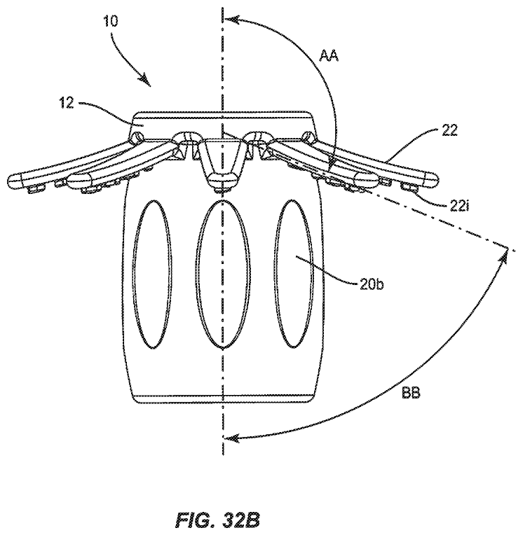

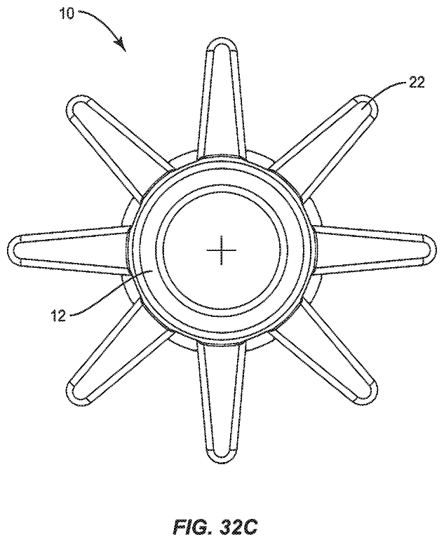

Referring to FIGS. 32A-32C, FIG. 32A illustrates a perspective view of an embodiment of a medical scoping device fitting 10 in accordance with the principles of the present disclosure. The fitting 10 illustrated is a single cylindrical member with a row of protuberances 22 spreading evenly near the distal end 12 of the cylindrical member 20. Each protuberance has a top surface 25 and a bottom surface 23. The bottom surface 23 includes multiple raised surfaces 22i extending perpendicularly or substantially perpendicularly to the bottom surface 23 of each protuberance. The raised surfaces 22i can be angled relative to each other on the bottom surface of the protuberance in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The raised surfaces 22i provide better friction and engagement of the inner lining of the colon tissue that aid in viewing tissue in the folds of the lining of the colon. The protuberances 22 are wider closer to the body and become narrower when approaching the outer edge of the protuberance.

The fitting can have raised surfaces 18a, which also can be angled relative to each other, the longitudinal axis or the rib 18 in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The angles of the raised surfaces 18a do not vary as the device is moved. This is unlike the angle of the protuberances as the fitting is moved, which change as the protuberances contact the lining of the colon. The raised surfaces 18a provide contact points with the head of the endoscope and allow the fitting 10 to conform to the surface of the endoscope and provide a friction or snug fit. The ribs 18 can run longitudinally in the interior of the cylindrical member and provide further support for the fitting 10. The ribs 18 and the raised surfaces 18a can be arranged in a pattern between alternating raised surfaces and ribs longitudinally arrayed in the interior of the fitting 10.

The cylindrical member includes multiple cavities 20b on its exterior. These cavities, in some embodiments, are oval shapes so that as the protuberance is compressed along the body in a downward direction, which can happen when the device enters the colon, the protuberance can engage cavities 20b to further stabilize the fitting 10. In some embodiments, the protuberance can contact the cavity.

FIG. 32B illustrates a side view of an embodiment of a medical scoping device fitting shown in 32A. The protuberances are angled in the embodiment shown in a downward direction relative to the body, where the angle AA from the top of the longitudinal axis of the fitting to the top of the protuberance is from about 95 degrees to about 175 degrees and the angle BB from the bottom of the protuberance to the bottom of the fitting is from about 5 degrees to about 85 degrees.

In some embodiments, the protuberances are positioned at an angle of from about 5°, 10°, 15°, 20°, 25°, 30°, 35°, 40°, 45°, 50°, 55°, 60°, 65°, 70°, 75°, 80°, or 85° in a downward direction relative to the body, when the fitting is at a resting position (e.g., there is no external force applied to it by the lining of the biological lumen). These angles can vary as the protuberances are at their rest position, their circumferentially inward position, and in their circumferentially outward position. The protuberances of the fitting can, in some embodiments, have a length, width and depth that is the same as shown in FIG. 32A.

FIG. 32C illustrates a top view of a medical scoping device fitting shown in 32A.

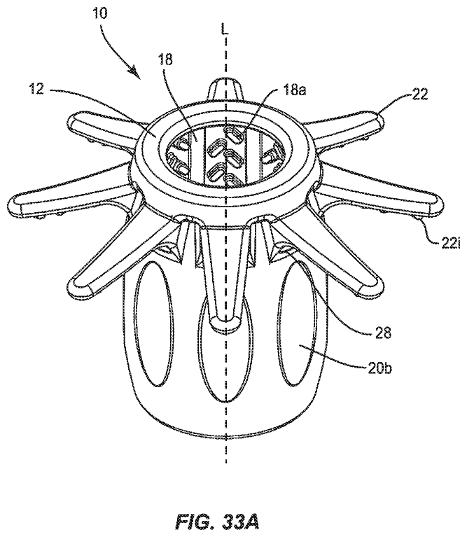

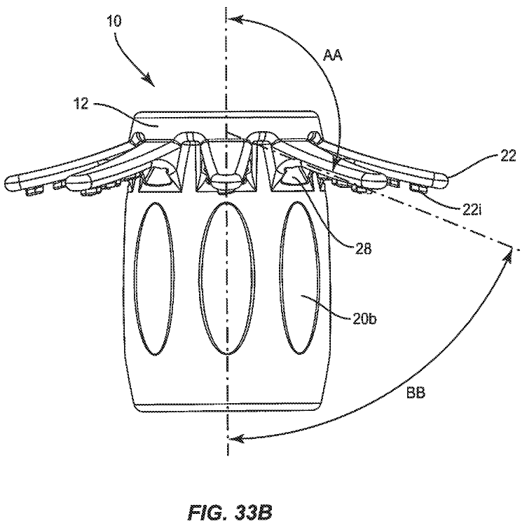

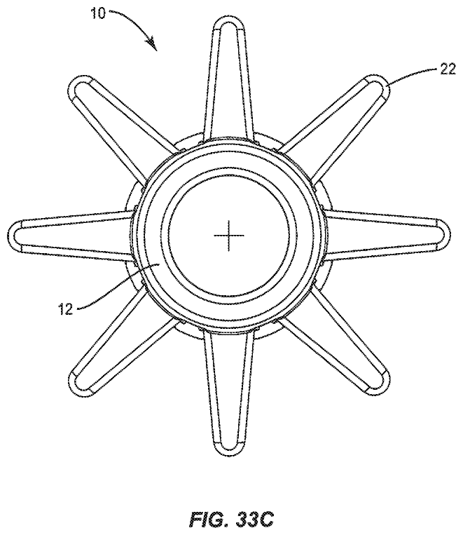

Referring to FIGS. 33A-33C, FIG. 33A illustrates a perspective view of an embodiment of a medical scoping device fitting 10 in accordance with the principles of the present disclosure. The fitting illustrated comprises a single cylindrical member with a row of protuberances 22 spreading evenly near the distal end 12 of the cylindrical member. Each protuberance is spaced apart and circumferentially arrayed with respect to one another and extending from the cylindrical member, each of the protuberances having an inner end and an outer edge to engage tissue. The inner end of each protuberance comprises a reinforced region 28. In some embodiments, the reinforced region has a peak von Mises stress or tensile stress of from about 3.2×10³ to about 9.8×10³ psi or 5.2×10³ to about 9.5×10³ psi or 7.2×10³ to about 8.868×10³ psi. The reinforced region 28 in the embodiment shown can be a region that has increased thickness relative to the middle portion or outer edge of the protuberance. This allows a greater degree of flexibility by the middle portion and the outer edge. Typically, the reinforced region is stiffer, thicker and less flexible than the middle portion and/or outer edge of the protuberance.

Each protuberance also includes multiple raised surfaces 22i extended perpendicularly from each protuberance. The raised surfaces 22i provide better friction and engagement of the inner lining of the colon tissue that aid in viewing tissue in the folds of the lining of the colon. The protuberances 22 are wider closer to the body by the reinforced region 28 and become narrower when approaching the outer edge of the protuberance. The reinforced regions can be any shape including oval, quadrilateral, or bubble shape.

The cylindrical member includes multiple cavities 20b on exterior walls and inner walls with ribs 18 and a pattern of raised surfaces 18a between two ribs. These cavities, in some embodiments, correspond to the shape of the protuberance so that as the protuberance is compressed along the body in a downward direction, the protuberance can engage cavities 20b to further stabilize the fitting 10. The cavities 20b can be any shape including, but not limited to, oval, quadrilateral, bubble shapes or a shape corresponding to the shape of the protuberance. In some embodiments, the protuberance can contact the cavity.

FIG. 33B illustrates a side view of an embodiment of a medical scoping device fitting shown in 33A. The protuberances are angled in the embodiment shown in a downward direction relative to the cylindrical body, where the angle AA from the top of the longitudinal axis of the fitting to the top of the protuberance is from about 95 degrees to about 175 degrees and the angle BB from the bottom of the protuberance to the bottom of the fitting is from about 5 degrees to about 85 degrees.

In some embodiments, the protuberances are positioned at an angle of from about 5°, 10°, 15°, 20°, 25°, 30°, 35°, 40°, 45°, 50°, 55°, 60°, 65°, 70°, 75°, 80°, or 85° in a downward direction relative to the body, when the fitting is at a resting position (e.g., there is no external force applied to it by the lining of the biological lumen). These angles can vary as the protuberances are at their rest position, their circumferentially inward position, and in their circumferentially outward position.

FIG. 33C illustrates a top view of an embodiment of a medical scoping device fitting shown in 33A.

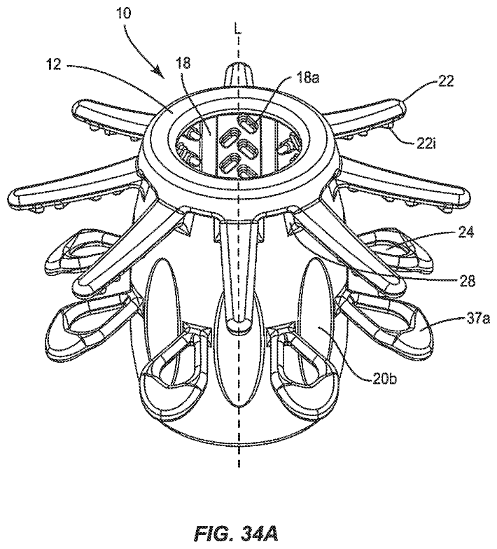

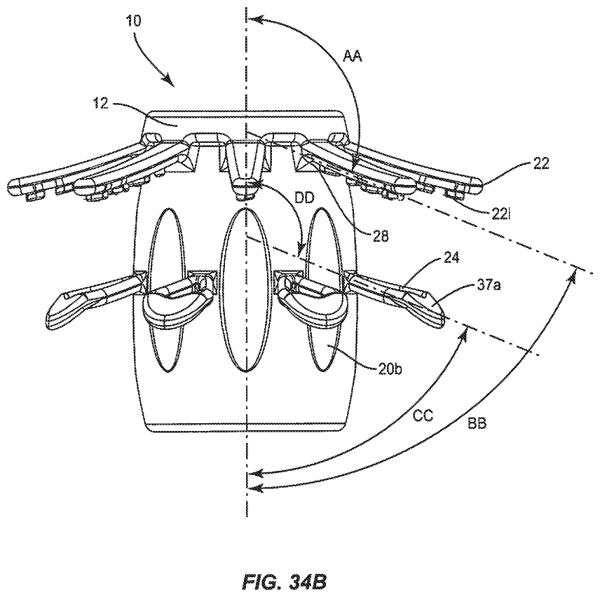

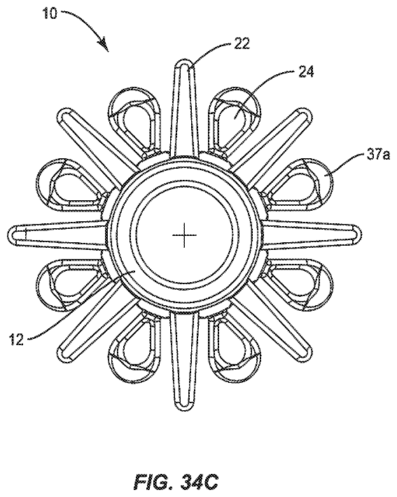

Referring to FIGS. 34A-34C, FIG. 34A illustrates a perspective view of an embodiment of a medical scoping device fitting 10 in accordance with the principles of the present disclosure. The fitting illustrated is a single cylindrical member with two rows of protuberances 22 with one row spreading evenly near the distal end 12 and another row spreading evenly around a lower section than the distal end of the cylindrical member 20. Each protuberance 22 near the distal end 12 includes multiple raised surfaces 22i extending perpendicularly from each protuberance. The raised surfaces on the bottom of the protuberance stabilize the fitting and helps slow movement of colon so protuberances do not snap back.

Each protuberance near the distal end 12 also includes a reinforced portion 28. The reinforced regions can be any shape including oval, quadrilateral, or bubble shape. Each protuberance 22 around the lower section of the cylindrical member includes a window 24 disposed between the outer edge and inner edge, a sloped portion 37a toward outer edge of the protuberance. The window 24 stabilizes the fitting 10 without blocking material that can pass through. In some embodiments, the sloped portioned 37a near the outer edge can have an angle of from about 90°, downward perpendicular to the protuberance to about 180°, flashing with the rest of the protuberance. The cylindrical member includes multiple cavities 20b on exterior walls 17 and inner walls with ribs 18 and a pattern of raised surfaces 18a between two ribs. The cavities 20b can be any shape including, but not limited to, oval, quadrilateral, bubble shapes or a shape corresponding to the shape of the protuberance. In some embodiments, the protuberance can contact the cavity. The fitting 10 has two rows of protuberances shown.

FIG. 34B illustrates a side view of an embodiment of a medical scoping device fitting shown in 34A. The protuberances are angled in the embodiment shown in a downward direction relative to the body, where the angle AA from the top of the longitudinal axis of the fitting to the top of the protuberance is from about 95 degrees to about 175 degrees, the angle BB from the bottom of the protuberance to the bottom of the fitting is from about 5 degrees to about 85 degrees, the angle DD from the top of the longitudinal axis of the fitting to the top of second row of the protuberance is from about 95 degrees to about 175 degrees, the angle CC from the bottom of the second row of the protuberance to the bottom of the fitting is from about 5 degrees to about 85 degrees.

In some embodiments, the protuberances are positioned at an angle of from about 5°, 10°, 15°, 20°, 25°, 30°, 35°, 40°, 45°, 50°, 55°, 60°, 65°, 70°, 75°, 800, or 85° in a downward direction relative to the body, when the fitting is at a resting position (e.g., there is no external force applied to it by the lining of the biological lumen). These angles can vary as the protuberances are at their rest position, their circumferentially inward position, and in their circumferentially outward position.

As shown, the first row of protuberances is not longitudinally aligned with the second row of protuberances having a window 24. Thus, the first row of protuberances 22 can have a staggered conformation with the second row of protuberances. The protuberances as shown can longitudinally align with the cavities 20b. The cavities 20b can alternate with the second row of protuberances having a window 24, when they are circumferentially arrayed about the body of the fitting.

FIG. 34C illustrates a top view of a medical scoping device fitting shown in 34A. The first row of protuberances 22 can alternate circumferentially about the body of the fitting 10 with the second row of protuberances having a window 24.

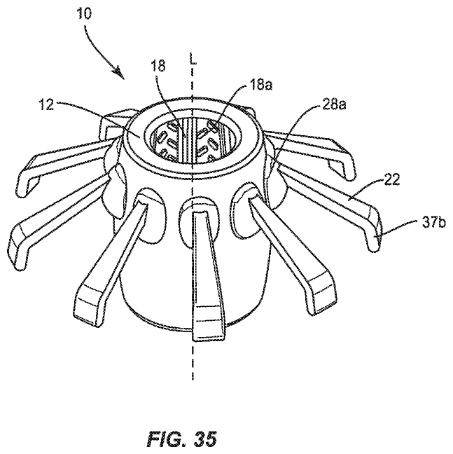

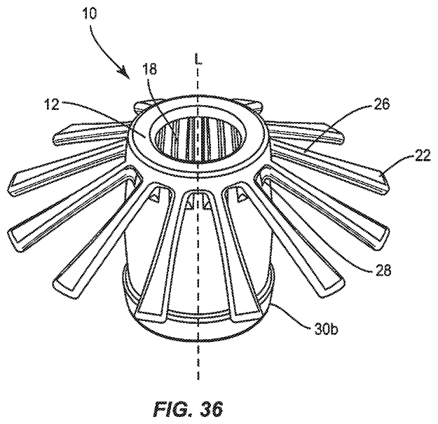

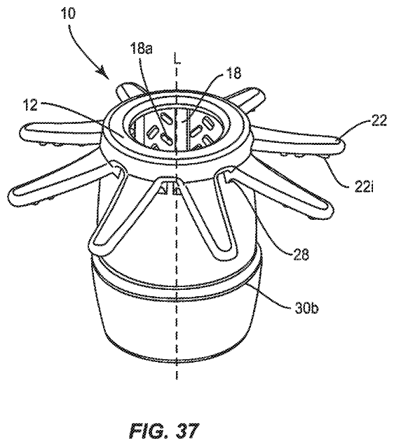

FIG. 35 illustrates a perspective view of an embodiment of a medical scoping device fitting 10 in accordance with the principles of the present disclosure. The fitting illustrated is a single cylindrical member with a row of protuberances 22 spreading evenly near the distal end 12 of the cylindrical member. Each protuberance is spaced apart and circumferentially arrayed with respect to one another and extending from the cylindrical member, each of the protuberances having an inner end and an outer edge to engage tissue. The inner end of each protuberance comprises a reinforced region. In some embodiments, the reinforced region has a peak von Mises stress or tensile stress of from about 3.2×10³ to about 9.8×10³ psi or 5.2×10³ to about 9.5×10³ psi or 7.2×10³ to about 8.868×10³ psi. The reinforced region in the embodiment shown can be a region that has increased thickness relative to the middle portion or outer edge of the protuberance. This allows a greater degree of flexibility by the middle portion and the outer edge. Typically, the reinforced region is stiffer, thicker and less flexible than the middle portion and/or outer edge of the protuberance. The reinforced regions can be any shape including oval, quadrilateral, or bubble shape 28a.

Each protuberance includes a sloped portion 37b toward the outer edge of the protuberance. In some embodiments, a sloped portioned near the outer edge can have an angle of from about 90°, downward perpendicular to the protuberance to about 180°, flashing with the rest of the protuberance. The raised surfaces 18a provide contact points with the head of the endoscope and allow the fitting 10 to conform to the surface of the endoscope and provide a friction or snug fit. The ribs 18 can run longitudinally in the interior of the cylindrical member and provide further support for the fitting 10. The ribs 18 and the raised surfaces 18a can be arranged in a pattern between alternating raised surfaces and ribs longitudinally arrayed in the interior of the fitting 10.

Referring to FIG. 36, FIG. 36 illustrates a perspective view of an embodiment of a medical scoping device fitting (e.g., endoscope) in accordance with the principles of the present disclosure. The fitting 10 illustrated is a single cylindrical body including a row of protuberances 22 spreading evenly near the distal end 12 of the cylindrical member. Each protuberance is spaced apart and circumferentially arrayed with respect to one another and extending from the cylindrical member, each of the protuberances having an inner end and an outer edge to engage tissue. The inner end of each protuberance comprises a reinforced region 28. The reinforced region 28 in the embodiment shown can be a region that has increased thickness relative to the middle portion or outer edge of the protuberance. This allows a greater degree of flexibility by the middle portion and the outer edge. Typically, the reinforced region is stiffer, thicker and less flexible than the middle portion and/or outer edge of the protuberance. The reinforced regions can be any shape including square, oval, quadrilateral, or bubble shape.

In some embodiments, the protuberance includes a narrowed portion 26 comprising a middle portion and the inner end of the protuberance has decreased width or surface area relative to the outer edge of the protuberance. The fitting also includes a wider and thicker region 30b at the proximal end. The thicker region being at the proximal portion compared to the middle portion and distal portion. This allows better engagement and stability when removably attached to the device. The inner wall of the fitting 10 includes ribs 18.

FIG. 37 illustrates a perspective view of an embodiment of a medical scoping device fitting (e.g., endoscope) in accordance with the principles of the present disclosure. The fitting illustrated is a single cylindrical body and includes a row of protuberances 22 spreading evenly near the distal end 12 of the cylindrical member. Each protuberance is spaced apart and circumferentially arrayed with respect to one another and extending from the cylindrical member, each of the protuberances having an inner end and an outer edge to engage tissue. The inner end of each protuberance comprises a reinforced region 28. The reinforced region 28 in the embodiment shown can be a region that has increased thickness relative to the middle portion or outer edge of the protuberance. This allows a greater degree of flexibility by the middle portion and the outer edge. Typically, the reinforced region is stiffer, thicker and less flexible than the middle portion and/or outer edge of the protuberance. The reinforced regions can be any shape including oval, quadrilateral, or bubble shape 28a.

In some embodiments, the protuberance comprises a middle portion and the inner end of the protuberance has the same or increased width or surface area relative to the middle portion and the outer edge of the protuberance. The fitting 10 also includes protuberances having raised surfaces 22i extending perpendicularly from each protuberance, a thicker portion 30b in the cylindrical body where the thicker region being at the middle portion compared to the distal portion and the proximal portion, and inner walls having ribs 18 and a pattern of raised surfaces 18a between two ribs.

Referring to FIGS. 38A-38C, FIG. 38A illustrates a perspective view of an embodiment of a medical scoping device fitting 10 in accordance with the principles of the present disclosure. The fitting 10 illustrated is a single elongated cylindrical member comprising a row of elongated protuberances 22 circumferentially arrayed near the distal end 12 of the elongated cylindrical member/body 20. The elongated protuberances 22 are flexible and have an elongated or extended length to increase visibility during a procedure. For example, elongated protuberances 22 facilitate increased visibility by passing through debris and unfolding folds present in the lumen wall away from the medical scoping device. Each elongated protuberance has a top surface 25 and a bottom surface 23. The bottom surface 23 is smooth, as shown in FIGS. 38A and 38B. The elongated protuberances 22 are wider closer to the body and become narrower when approaching an outer edge 29 of the elongated protuberance. Elongated protuberances 22 are arranged in a row and extend outward from elongated cylindrical member 20 between an inner end 27 and the outer edge 29. In some embodiments, each elongated protuberance 22 has a varied degree of flexibility from the inner end 27 to the outer edge 29. In some embodiments, elongated protuberance 22 does not contain a hinge. In some embodiments, the outer edge 29 is contoured to a smooth finish, as shown in FIG. 38A, so as to reduce trauma to tissue.

In some embodiments, the elongated protuberances 22 individually and independently move so as to allow for a smooth removal of the medical scoping device from the body passage and orifice into which the medical scoping device has been inserted. In some embodiments, the elongated protuberances 22 move together and do not move individually and independently.

In some embodiments, the elongated cylindrical member 20 can have a length of from about 4 mm to about 50 mm. In various embodiments, each of the protuberances can be from about 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm or about 50 mm in length.

In some embodiments, the elongated protuberances 22 are between about 2 to about 40 mm in length from the inner end 27 to the outer edge 29. In some embodiments, the elongated protuberances 22 can have a length between about 2 to about 5 mm, between about 4 to about 18 mm, between about 7 to about 16 mm, between about 10 to about 15 mm, between about 13 to about 18 mm, between about 16 to about 21 mm, between about 19 to about 24 mm, between about 21 to about 27 mm, between about 24 to about 30 mm, between about 27 to about 33 mm, between about 30 to about 37 mm or between about 33 to about 40 mm. In various embodiments, each of the elongated protuberances 22 can be from about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm or about 40 mm in length.

In various embodiments, each of the elongated protuberances have a width of from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. The width can vary throughout the fitting and/or along the elongated protuberance 22 to achieve the desired flexibility.

The fitting 10 can have raised surfaces 18a, which also can be angled relative to each other, the longitudinal axis or the rib 18 in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The angles of the raised surfaces 18a do not vary as the device is moved. This is unlike the angle of the elongated protuberances 22 as the fitting is moved, which change as the elongated protuberances 22 contact the lining of the colon. The raised surfaces 18a provide contact points with the head of the endoscope and allow the fitting 10 to conform to the surface of the endoscope and provide a friction or snug fit. The ribs 18 can run longitudinally in the interior of the elongated cylindrical member and provide further support for the fitting 10. The ribs 18 and the raised surfaces 18a can be arranged in a pattern between alternating raised surfaces and ribs longitudinally arrayed in the interior of the fitting 10.

The elongated cylindrical member 20 includes multiple cavities 20b on its exterior. These cavities, in some embodiments, are oval shapes so that as the elongated flexible protuberance is compressed along the body in a downward direction, which can happen when the device enters the colon, the elongated flexible protuberance 22 can engage cavities 20b to further stabilize the fitting 10. In some embodiments, the elongated flexible protuberance 22 can contact the cavity. In this embodiment, the protuberances are longer and narrower to increase visibility between the user and the view of the colon.

FIG. 38B illustrates a side view of an embodiment of a medical scoping device fitting shown in 38A. The elongated protuberances 22 are angled in the embodiment shown in a downward direction relative to the body. In some embodiments, the elongated protuberances 22 are positioned at an angle of from about 5°, 10°, 15°, 20°, 25°, 30°, 35°, 40°, 45°, 50°, 55°, 60°, 65°, 70°, 75°, 80°, or 85° in a downward direction relative to the body, when the fitting 10 is at a resting position (e.g., there is no external force applied to it by the lining of the biological lumen). These angles can vary as the elongated protuberances are at their rest position, their circumferentially inward position, and in their circumferentially outward position. The elongated protuberances of the fitting can, in some embodiments, have a width and depth that is the same as shown in FIG. 32A.

FIG. 38C illustrates a top view of a medical scoping device fitting shown in 38A.

Referring to FIGS. 38D-38G, FIG. 38D illustrates a perspective view of an embodiment of a medical scoping device fitting 10, as shown in FIG. 38A in accordance with the principles of the present disclosure. The fitting 10 includes a tab 300 extending from a proximal end 14 of fitting 10. The fitting 10 and/or the tab 300 includes a perforation line 304 extending between a proximal end 306 and a distal end 308 that is disposed on the elongated cylindrical member/body 20. The tab 300 is disposed on the elongated body of the fitting and aligned along the longitudinal axis. Distal end 308 terminates at elongated cylindrical member 20 and proximal end 306 terminates at proximal end 14 of fitting 10.

In some embodiments, the fitting and/or the tab 300 includes a first perforation line 304 and a second perforation line 304. First perforation line 304 is parallel to second perforation line 304, and they are disposed on opposing sides of cavity 20b. In some embodiments, fitting 10 and/or tab 300 do not include a perforation line 304. In some embodiments, fitting 10 comprises a first tab 300 and a second tab 300, as shown in FIG. 38G, where the second tab 300 is disposed opposite first tab 300. In some embodiments, fitting 10 comprises 1, 2, 3, 4 or 5 tabs 300.

Tab 300 includes a handle, such as, for example, projection 302 that extends outwardly, but not at a distance further than the elongated protuberances 22 in an uncompressed configuration, from proximal end 14 of fitting 10. Tab 300 is gripped by a user's thumb and fingers at projection 302 to assist in assembling fitting 10 on the endoscope and removing fitting 10 from the endoscope. Tab 300 is torn and removed in one motion in an upward and distal direction (e.g., away from the endoscope) by the user to facilitate removal of fitting 10 from the endoscope. Thus, the fitting has a tearable section or strip that allows the user to remove and destroy the fitting so that it is single use.

In some embodiments, tab 300 can run continuously throughout the entire fitting 10 in a circumferentially arrayed manner with elongated protuberances 22, or at discrete position of the proximal end 14 of fitting 10 as shown in FIG. 38D. In some embodiments, tab 300 is monolithic with fitting 10. In some embodiments, tab 300 is separate from fitting 10 and attached to fitting 10 in a manner so as to not interfere with protuberances 22. In some embodiments, tab 300 has a thickness that is greater or less than a thickness of elongated protuberances 22, elongated cylindrical member/body 20, and/or fitting 10. In some embodiments, tab 300 has a thickness of about 1 mm to about 10 mm. In some embodiments, tab 300 has a thickness of about 1 mm, 2 mm, 3 mm, 4 mm, 5 mm, 6 mm, 7 mm, 8 mm, 9 mm or 10 mm. In some embodiments, tab 300 has a thickness that is less than a thickness of elongated protuberances 22, elongated cylindrical member/body 20, and/or fitting 10 to provide a weakness for tearing and removal of tab 300 from fitting 10.

In some embodiments, perforation line 304 includes embedded materials, such as polymers to allow tearing of cylindrical member/body 20 or tab 300 along the longitudinal axis of fitting 10.

In some embodiments, perforation line or lines 304 provide a tear line along the cylindrical member/body 20 of fitting 10 or tab 300 to allow the user to remove fitting 10 from the endoscope, and fitting 10 will be permanently destroyed, and therefore, be a single use only. This avoids the possibility of inadvertent re-use of the fitting 10 to prevent cross-contamination with different patients. This will also allow the fitting 10 to be used with a specific endoscope and it can be tracked with that specific endoscope. In some embodiments, fitting 10 or tab 300 allows the user to remove fitting 10 from the endoscope without the fitting 10 or tab 300 being damaged or destructively removed.

Referring to FIGS. 38H-38L, FIG. 38H illustrates a perspective view of an embodiment of a medical scoping device fitting 10, as shown in FIG. 38A in accordance with the principles of the present disclosure. The fitting 10 includes a tab 400. Fitting 10 or tab 400 comprise a wall 404 extending from a proximal end 406 and a distal end 408. Distal end 408 terminates at distal end 12 of elongated cylindrical member/body 20 and proximal end 406 terminates at proximal end 14. Wall 404 is disposed along the elongated cylindrical member/body 20 and the longitudinal axis, and wall 404 has a thickness that is less than a thickness of the cylindrical member/body 20 to facilitate removal of fitting 10 from the endoscope. Examples of a wall include, but are not limited to a flash, a line, or a ridge.

In some embodiments, fitting 10 or tab 400 comprises a first wall 404 and a second wall 404 disposed along the elongated cylindrical member/body 20 and the longitudinal axis. In some embodiments, the first wall 404 and the second wall 404 have a thickness that is less than a thickness of the elongated cylindrical member/body 20 to facilitate removal of fitting 10 from the endoscope. In some embodiments, first wall 404 is parallel to second wall 404, and they are disposed on opposing sides of cavity 20b. These walls can make up the tearable section or strip that allows the user to remove and destroy the fitting so that it is single use.

In some embodiments, fitting 10 comprises a first tab 400 and a second tab 400, as shown in FIG. 38L, where the second tab 400 is disposed opposite the first tab 400. In some embodiments, fitting 10 comprises 1, 2, 3, 4 or 5 tabs 400.

In some embodiments, wall 404 or walls 404 are 5, 4, 3, 2 or 1 times less thick than the thickness of the elongated cylindrical member/body 20. In some embodiments, tab 400 has a thickness of about 1 mm to about 10 mm. In some embodiments, tab 400 has a thickness of about 1 mm, 2 mm, 3 mm, 4 mm, 5 mm, 6 mm, 7 mm, 8 mm, 9 mm or 10 mm. In some embodiments, tab 400 has a thickness that is less than a thickness of elongated protuberances 22, elongated cylindrical member/body 20, and/or fitting 10 to provide a weakness for tearing and removal of tab 400 from fitting 10.

Tab 400 includes a handle, such as, for example, projection 402 that extends outwardly, but not at a distance further than the elongated protuberances 22 in an uncompressed configuration, from proximal end 14 of fitting 10. Tab 400 is gripped by a user's thumb and fingers at projection 402 to assist in applying and removing fitting 10 from an endoscope. Tab 400 is torn and removed in one motion in an upward and distal direction by the user to facilitate removal of fitting 10 from the endoscope. In some embodiments, tab 400 is peeled from fitting 10.

In some embodiments, tab 400 can run continuously throughout the entire fitting 10 in a circumferentially arrayed manner with elongated protuberances 22, or at discrete position of the proximal end 14 of fitting 10 as shown in FIG. 38H. In some embodiments, tab 400 is monolithic with fitting 10. In some embodiments, tab 400 is separate from fitting 10 and attached to fitting 10 in a manner so as to not interfere with protuberances 22. In some embodiments, tab 400 has a thickness that is greater or less than a thickness of elongated protuberances 22, elongated cylindrical member/body 20, and/or fitting 10. In some embodiments, tab 400 has a thickness of about 1 mm to about 10 mm. In some embodiments, tab 400 has a thickness of about 1 mm, 2 mm, 3 mm, 4 mm, 5 mm, 6 mm, 7 mm, 8 mm, 9 mm or 10 mm. In some embodiments, tab 400 has a thickness that is less than a thickness of elongated protuberances 22, elongated cylindrical member/body 20, and/or fitting 10 to provide a weakness for tearing and removal of tab 400 from fitting 10.

In some embodiments, wall 404 includes embedded materials, such as polymers to allow tearing of cylindrical member/body 20 or tab 400 along the longitudinal axis of fitting 10.

In some embodiments, wall or walls 404 provide a tear line along the cylindrical member/body 20 of fitting 10 or tab 400 to allow the user to remove fitting 10 from the endoscope, and fitting 10 will be permanently destroyed, and therefore, be a single use only. This avoids the possibility of inadvertent re-use of the fitting 10 to prevent cross-contamination with different patients. This will also allow the fitting 10 to be used with a specific endoscope and it can be tracked with that specific endoscope. In some embodiments, fitting 10 or tab 400 allows the user to remove fitting 10 from the endoscope without the fitting 10 or tab 400 being damaged or destructively removed.

Referring to FIG. 38M, FIG. 38M illustrates a side view of an embodiment of a medical scoping device fitting 10, as shown in FIG. 38A in accordance with the principles of the present disclosure. Fitting 10 includes an embedded plastic strip or strips 600 that act similarly to tabs 300 and 400. In some embodiments, the embedded strip or strips 600 are disposed along the elongated body and the longitudinal axis of fitting 10. In some embodiments, the strip or strips 600 are ripped off the body to facilitate removal of fitting 10 from the endoscope. The strip or strips 600 are engaged and ripped in an upward and distal direction by a user to facilitate removal of fitting 10 from the endoscope. In some embodiments, the strip or strips 600 are fabricated from various plastics or thread of various lengths and thicknesses.

In some embodiments, the strip or strips 600 can run continuously throughout the entire fitting 10 in a circumferentially arrayed manner with elongated protuberances 22, or at discrete positions of fitting 10. In some embodiments, the strip or strips 600 are monolithic with fitting 10. In some embodiments, the strip or strips 600 are separate from fitting 10 and attached to fitting 10 in a manner so as to not interfere with protuberances 22.

In some embodiments, the strip or strips 600 includes embedded materials, such as polymers to allow tearing of cylindrical member/body 20 or strips 600 along the longitudinal axis of fitting 10.

In some embodiments, the strip or strips 600 provide a tear line along the cylindrical member/body 20 of fitting 10 or strips to allow the user to remove fitting 10 from the endoscope, and fitting 10 will be permanently destroyed, and therefore, be a single use only. This avoids the possibility of inadvertent re-use of the fitting 10 to prevent cross-contamination with different patients. This will also allow the fitting 10 to be used with a specific endoscope and it can be tracked with that specific endoscope. In some embodiments, fitting 10 comprises a first strip 600 and a second strip 600, where the second strip 600 is disposed opposite the first strip 600. In some embodiments, fitting 10 comprises 1, 2, 3, 4 or 5 strips 600.

Referring to FIG. 38N, FIG. 38N illustrates a perspective view of an embodiment of a medical scoping device fitting 10, as shown in FIG. 38A in accordance with the principles of the present disclosure. The fitting 10 includes a tube 500 that allows enhanced viewing of the procedure site. Tube 500 is disposed on distal end 12 of fitting 10. In some embodiments, tube 500 is fused to distal end 12 of fitting 10. In some embodiments, tube 500 is monolithic or adhered to fitting 10 with an adhesive. In some embodiments, tube 500 is dome shaped and has a smooth finish to reduce trauma to the tissue. Tube 500 comprises a proximal end 502 and a distal end 504. Proximal end 502 engages distal end 12 of fitting 10. Tube 500 is configured to assist a user in navigating a procedure site. For example, elongated protuberances 22 are spaced a distance from tube 500 to engage and view an endoscopic mucosal resection (EMR) or an ileum intubation. Tube 500 can be transparent and in some embodiments, is a clear plastic material. In some embodiments, tube 500 has a length of about 1 mm, 2 mm, 3 mm, 4 mm, 5 mm, 6 mm, 7 mm, 8 mm, 9 mm to 10 mm. In some embodiments, tube 500 has a length of about 1 mm-8 mm. In some embodiments, tube 500 has a length of about 1 mm to about 4 mm, about 1 mm to about 6 mm, about 1 mm to about 8 mm, or about 1 to about 10 mm.

Referring to FIGS. 39A-38C, FIG. 39A illustrates a perspective view of an embodiment of a medical scoping device fitting 10 in accordance with the principles of the present disclosure similar to FIG. 38A-38C. The fitting 10 illustrated includes is a single elongated cylindrical member with a row of elongated protuberances 22 circumferentially arrayed near the distal end 12 of the elongated cylindrical member 20. Elongated protuberances 22 are flexible and have an elongated or extended length to increase visibility during a procedure. For example, elongated protuberances 22 facilitate increased visibility by passing through debris and unfolding folds present in the lumen wall away from the medical scoping device.

In some embodiments, the elongated protuberances 22 individually and independently move so as to allow for a smooth removal of the medical scoping device from the body passage and orifice into which the medical scoping device has been inserted. In some embodiments, the elongated protuberances 22 move together and do not move individually and independently.

Each elongated protuberance 22 has a top surface 25 and a bottom surface 23. The bottom surface 23 includes multiple raised surfaces comprising a first raised surface 22i and a second raised surface 22i disposed on a distal end/outer edge 29 of the elongated protuberance 22. In some embodiments, the raised surfaces 22i extend perpendicularly or substantially perpendicularly to the bottom surface 23 of each elongated protuberance 22. The raised surfaces 22i can be angled relative to each other on the bottom surface 23 of the elongated protuberance 22 in a fish bone style or at an angle of from about 5°, 10°, 15°, 20°, 25°, 30°, 35°, 40°, 45°, 50° 55°, 60°, 65°, 70°, 75°, 80°, 85°, or to about 90°. The raised surfaces 22i provide better friction and engagement of the inner lining of the colon tissue that aid in viewing tissue in the folds of the lining of the colon.

The raised surfaces 22i are rounded to avoid trauma, and in some embodiments, the raised surfaces 22i are bumps. In some embodiments, raised surfaces 22i are configured into various shapes such as ovals, circles, squares, rectangles, triangles, irregularly shaped, and any combination thereof.

The elongated protuberances 22 are wider closer to the body and become narrower when approaching the outer edge 29 of the elongated protuberance 22. Elongated protuberances 22 are arranged in a row and extend outward from elongated cylindrical member 20 between an inner end 27 and the outer edge 29. In some embodiments, each elongated protuberance 22 has a varied degree of flexibility from the inner end 27 to the outer edge 29. The fitting 10 can have raised surfaces 18a, and rib 18 similar to that described above with regard to FIGS. 38A-38C. In some embodiments, the outer edge 29 is contoured to a smooth finish, as shown in FIG. 39A, so as to reduce trauma to tissue.

Referring to FIGS. 40A-40C, FIG. 40A illustrates a perspective view of an embodiment of a medical scoping device fitting 10, in accordance with the principles of the present disclosure similar to FIGS. 38A-38C. The fitting 10 illustrated is a single elongated cylindrical member with a row of elongated and narrow protuberances 22 circumferentially arrayed near the distal end 12 of the elongated cylindrical member 20. The elongated and narrow protuberances 22 are positioned lower on the elongated cylindrical member 20 than the elongated protuberances shown in FIGS. 38A-38C. The elongated and narrow protuberances 22 are flexible and have an elongated or extended length to increase visibility during a procedure. For example, elongated and narrow protuberances 22 facilitate increased visibility by passing through debris and unfolding folds present in the lumen wall away from the medical scoping device.

In some embodiments, the elongated and narrow protuberances 22 individually and independently move so as to allow for a smooth removal of the medical scoping device from the body passage and orifice into which the medical scoping device has been inserted. In some embodiments, the elongated and narrow protuberances 22 move together and do not move individually and independently.

Each elongated and narrow protuberance has a top surface 25 and a bottom surface 23. The bottom surface 23 is smooth, as shown in FIGS. 40A and 40B. The elongated and narrow protuberances 22 are wider closer to the body and become narrower when approaching an outer edge 29 of the elongated and narrow protuberance 22. Elongated and narrow protuberances 22 are arranged in a row and extend outward from elongated cylindrical member 20 between an inner end 27 and the outer edge 29. In some embodiments, each elongated and narrow protuberance 22 has a varied degree of flexibility from the inner end 27 to the outer edge 29. In some embodiments, the outer edge 29 is contoured to a smooth finish, as shown in FIG. 40A, so as to reduce trauma to tissue.

In some embodiments, the elongated and narrow protuberances 22 are between about 2 to about 40 mm in length from the inner end 27 to the outer edge 29. In some embodiments, the elongated and narrow protuberances 22 can have a length between about 2 to about 5 mm, between about 4 to about 18 mm, between about 7 to about 16 mm, between about 10 to about 15 mm, between about 13 to about 18 mm, between about 16 to about 21 mm, between about 19 to about 24 mm, between about 21 to about 27 mm, between about 24 to about 30 mm, between about 27 to about 33 mm, between about 30 to about 37 mm or between about 33 to about 40 mm. In various embodiments, each of the elongated and narrow protuberances 22 can be from about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm or about 40 mm in length.

In various embodiments, each of the elongated and narrow protuberances have a width of from about 0.25 mm, 0.5 mm, 0.75 mm, 1 mm, 1.25 mm, 1.5 mm, 1.75 mm, 2 mm, 2.25 mm, 2.5 mm, 2.75 mm, 3.0 mm, 3.25 mm, 3.5 mm, 3.75 mm, 4 mm, 4.25 mm, 4.5 mm, 4.75 mm, to about 5 mm in width. In some embodiments, each of the elongated and narrow protuberances 22 have a width of about 0.5 mm to about 4 mm, about 0.5 mm to about 3.0 mm, about 0.5 mm to about 2.0 mm, about 0.5 mm to about 1.0 mm, about 1.0 mm to about 4 mm, about 1.0 mm to about 3 mm, about 1.0 mm to about 2.0 mm, about 2.0 mm to about 4.0 mm, or about 2.0 mm to about 3.0 mm. The width can vary throughout the fitting and/or along the elongated and narrow protuberance 22 to achieve the desired flexibility. In some embodiments, fitting 10 is configured for use in a terminal ileum. The fitting 10 can have raised surfaces 18a, and rib 18 similar to that described above with regard to FIGS. 38A-38C.

Referring to FIGS. 41A-41C, FIG. 41A illustrates a perspective view of an embodiment of a medical scoping device fitting 10, in accordance with the principles of the present disclosure. The fitting illustrated is a single cylindrical member with two sets of protuberances 22 with a first set circumferentially arrayed near the distal end 12, and a second set circumferentially arrayed around a lower section of the cylindrical member/body 20. The first set of protuberances 22 include multiple raised surfaces comprising a first raised surface 22i and a second raised surface 22i extending perpendicularly from each protuberance, and disposed on a distal end/outer edge 29 of protuberance 22. In some embodiments, the outer edge 29 is contoured to a smooth finish, as shown in FIG. 41A, so as to reduce trauma to tissue.

The raised surfaces 22i on a bottom of the protuberance 22 stabilize the fitting and helps slow movement of the colon so that the protuberances 22 do not snap back. The raised surfaces 22i are rounded.

In some embodiments, each protuberance 22 individually and independently move so as to allow for a smooth removal of the medical scoping device from the body passage and orifice into which the medical scoping device has been inserted. In some embodiments, the protuberances 22 move together and do not move individually and independently.

Each protuberance 22 near distal end 12 also includes a reinforced portion 28. The reinforced regions can be any shape including oval, quadrilateral, or bubble shape. Each protuberance 22 in the second set around the lower section of the cylindrical member/body 20 includes a cut out or window 24 disposed between the outer edge 29 and an inner end 27, and a sloped portion 37a toward the outer edge 29 of protuberance 22. The window 24 stabilizes the fitting 10 without blocking material that can pass through. In some embodiments, the sloped portioned 37a near the outer edge 29 can have an angle of from about 90°, downward perpendicular to the protuberance to about 180°, flashing with the rest of the protuberance. The cylindrical member includes multiple cavities 20b on exterior walls and inner walls with ribs 18 and a pattern of raised surfaces 18a between two ribs. The cavities 20b can be any shape including, but not limited to, oval, quadrilateral, bubble shapes or a shape corresponding to the shape of the protuberance. In some embodiments, the protuberance can contact the cavity. The fitting 10 has two sets of protuberances 22 shown. The first and second sets of protuberances 22 are in a horizontal and parallel alignment relative to each other.

FIG. 41B illustrates a side view of an embodiment of a medical scoping device fitting shown in 41A. In some embodiments, the protuberances 22 are angled in the embodiment shown in a downward direction relative to the body, where the angle from the top of the longitudinal axis of the fitting to the top of the protuberance 22 is from about 95 degrees to about 175 degrees, the angle from the bottom of the protuberance 22 to the bottom of the fitting 10 is from about 5 degrees to about 85 degrees, the angle from the top of the longitudinal axis of the fitting to the top of second row of the protuberance 22 is from about 95 degrees to about 175 degrees, the angle from the bottom of the second row of the protuberance 22 to the bottom of the fitting is from about 5 degrees to about 85 degrees.

In some embodiments, the protuberances 22 are positioned at an angle of from about 5°, 10°, 15°, 20°, 25°, 30°, 35°, 40°, 45°, 50°, 55°, 60°, 65°, 70°, 75°, 80°, or 85° in a downward direction relative to the body, when the fitting is at a resting position (e.g., there is no external force applied to it by the lining of the biological lumen). These angles can vary as the protuberances are at their rest position, their circumferentially inward position, and in their circumferentially outward position.

In some embodiments, fitting 10 is provided for releasable attachment to a medical scoping device, the fitting comprising an elongated cylindrical member/body configured for disposal over the medical scoping device, the fitting extending along at least a portion of a length of a distal end of the medical scoping device, the elongated cylindrical member/body comprising an inner surface that grips at least a portion of the medical scoping device and holds the fitting in place, the elongated cylindrical member/body further comprising an outer surface comprising a plurality of flexible and resiliently deformable spaced apart protuberances having an inner end and an outer edge that are movable between a resting position to a position where the outer edge of the protuberances are substantially parallel to a longitudinal axis of the medical scoping device, and to a position that is at an angle approximately perpendicular to the longitudinal axis of the medical scoping device so that the protuberances are fanned out to contact with and provide support for and to dilate a lumen wall of a body passage into which the medical scoping device has been inserted, wherein the protuberances have a diameter of between 0.2 to 6.0 mm, and are movable beyond the angle approximately perpendicular to the longitudinal axis of the medical scoping device and flick over at a critical point of maximum inflexion so that the outer edges point towards the distal end of the medical scoping medical device, and the protuberances further comprise a first raised surface and a second raised surface disposed on the outer edge of the protuberance.

Referring to FIGS. 42A to 42D, FIG. 42A illustrates a perspective view of an embodiment of a medical scoping device fitting 10, in accordance with the principles of the present disclosure. The fitting 10 includes six protuberances 22 that extend radially and are spaced a part at a distance D₁ from the body of the medical scoping device to provide better access to the terminal ilium. In one embodiment, four of the six protuberances are moved 1.5 mm proximal from the distal end of the device. The protuberances 22 also include raised surfaces 22j disposed on a top surface of the protuberances 22 to provide better friction and engagement of the inner lining of the colon tissue that aid in viewing tissue in the folds of the lining of the colon.

Referring to FIGS. 43A to 43D, FIG. 43A illustrates a perspective view of an embodiment of a medical scoping device fitting 10, in accordance with the principles of the present disclosure. The fitting 10 includes six protuberances 22 that include raised surfaces 22j, similar to the protuberances shown in FIGS. 42A to 42D. The fitting 10 further includes a tab 700 extending from a proximal end 14 of fitting 10. The tab 700 is disposed on the elongated body of the fitting and aligned along the longitudinal axis L.

Tab 700 includes a handle, such as, for example, projection 702 that extends outwardly, but not at a distance further than the elongated protuberances 22 in an uncompressed configuration, from proximal end 14 of fitting 10. Tab 700 is gripped by a user's thumb and fingers at projection 702 to assist in assembling fitting 10 on the endoscope and removing fitting 10 from the endoscope. Tab 700 is torn and removed in one motion in an upward and distal direction (e.g., away from the endoscope) by the user to facilitate removal of fitting 10 from the endoscope. Thus, the fitting has a tearable section or strip that allows the user to remove and destroy the fitting so that it is single use.

In some embodiments, the fitting has various modulus of elasticity from the body, and/or the protuberances running from the inner end 27 to the outer edge 29 of the protuberance in the range of from about 0.01 GPa to about 1000 GPa, or 0.01 GPa to about 200 GPa, or 0.01 GPa to 0.1 GPa, or 0.1 GPa to 1 GPa, or 1 GPa to 10 GPa, or 10 GPa to 50 GPa, or 50 GPa to 100 GPa, or 1 MPa to about 50 MPa, or 1 MPa to about 5 MPa, or 5 MPa to about 10 MPa or 10 MPa to about 15 MPa, or 15 MPa to about 20 MPa, or 20 MPa to about 25 MPa, or 25 MPa to about 30 MPa, or 30 MPa to about 35 MPa, or 35 MPa to about 40 MPa or 40 MPa to about 45 MPa, or 45 MPa to about 50 MPa and different hardness in Shore A and Shore D of about 0-to about 100 durometers, or 0 durometers to about 5+/−4 durometers, or 5+/−5 durometers to about 10+/−5 durometers, or 10+/−5 durometers to about 15+/−5 durometers, or 15+/−5 durometers to about 20+/−5 durometers, or 20+/−5 durometers to about 25+/−5 durometers, or 25+/−5 durometers to about 30+/−5 durometers, or 30+/−5 durometers to about 35+/−5 durometers, or 35+/−5 durometers to about 40+/−5 durometers, or 40+/−5 durometers to about 45+/−5 durometers, or 45+/−5 durometers to about 50+/−5 durometers, or 50+/−5 durometers to about 55+/−5 durometers, 55+/−5 durometers to about 60+/−5 durometers, or 60+/−5 durometers to about 65+/−5 durometers, or 65+/−5 durometers to about 70+/−5 durometers, or 70+/−5 durometers to about 75+/−5 durometers, or 75+/−5 durometers to about 80+/−5 durometers, or 80+/−5 durometers to about 85+/−5 durometers, or 85+/−5 durometers to about 90+/−5 durometers, or 90+/−5 durometers to about 95+/−5 durometers. In some embodiments, the fitting has hardness in Shore A and Shore D of about 1, 5, 10, 15, 20, 25, 30, 35, 40, 45, 50, 55, 60, 65, 70, 75, 80, 85 or 90 durometers.

It will be understood by those of ordinary skill in the art that the body passage device and one or more of its components can be sterilized and reused. Alternatively, and more preferred, one or more components of the body passage device can be disposable and disposed of after single use.

It will be appreciated that the fitting of the present application can be constructed with various diameters so that it may be used to fit over the shaft of existing medical scoping devices. For example, pediatric scoping devices comprise shaft diameters of around 11 mm whereas an adult scoping device shaft diameter is in the region of 12 mm, the fitting of the present application may be constructed with suitable diameters according to a user's requirements.

It will be understood that each of the protuberances of the fitting of the present application can be constructed with various diameters. In some embodiments, the protuberances have a diameter of about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, and/or 10 mm. In some embodiments, the diameter can vary throughout the protuberance.

### Method of Use

In use, the disclosed fitting comprises protuberances which are configured to expand the lining of a biological lumen. Described herein is a method for using a device, such as fitting 10, with an endoscope to increase visualization of the colon or other biological lumens during endoscopic procedures. The fitting includes an array of protuberances which are configured to grasp onto and expand a biological lumen without incurring any damage to tissue. Prior to an endoscopic procedure, a medical practitioner obtains an endoscope and attaches fitting 10 at or near the distal tip of said endoscope. This can be done by hand or with a hand tool. The endoscope is passed into channel 16 of fitting 10 that is configured to receive an endoscope. Ribs 18 extend along the length of channel 16 and increase a friction fit between fitting 10 and the sidewall of an endoscope.

Once the endoscope is outfitted with fitting 10, it is inserted into the biological lumen of a patient. On inserting the endoscope, protuberances 22, 32 are moved from the rest position to a first position where they are flattened towards the proximal end of the fitting. When in the first position, fitting 10 has a thinner profile to enable an endoscope to easily be advanced through a biological lumen. During intubation, protuberances 22, 32 are configured to collapse into the device during insertion through an opening into a biological lumen, such as a sphincter. When advanced through the biological lumen, the angle at which protuberances 22, 32 are disposed with fitting 10 enables the endoscope to glide through the biological lumen with little resistance. Specifically, the leading row of protuberances may be angled more acutely in the proximal direction to assist in easy insertion. Additionally, the leading row of protuberances may include a shorter length than the trailing row of protuberances to minimize resistance of movement in the distal direction. In some embodiments, the leading row of protuberances is formed from a material which causes less friction against the lining of the biological lumen.

The flexible shaft of the endoscope is advanced distally through the biological lumen until a medical practitioner reaches a spot where increased visualization is desired. Once a medical examiner reaches a desired location, distal advancement of the endoscope stops. This causes protuberances 22, 32 to return to the rest position. The medical practitioner draws the endoscope proximally an amount as needed to cause a threshold amount of friction between the protuberances and the lining of the biological lumen. When the endoscope is retracted and moved in a proximal direction, the outer edges of the protuberances 22, 32 grip the lining of the lumen and flare outward to the second position. The outer edges of the protuberances are blunt and made from an elastomeric material so as to avoid damage to tissue during the transition from the first position to the second position. Once in the second position, tissue contacting surfaces on the underside of protuberances 22, 32 are configured to grip the lining of the biological lumen to anchor the endoscope in place. When in the second position, fitting 10 has a wider profile to facilitate anchoring into the lining of a biological lumen. The protuberances exert a mechanical force upon the lining of a biological lumen when in the second position so as to force apart any folds which are present in the lumen wall. Since protuberances 22, 32 are biased to the rest position, when the protuberances are in the second position, they are able to exert an outward force on the lining of the lumen and create a wider area for the endoscope to visualize.

Once expanded, the medical practitioner may visualize a portion of the biological lumen as necessary. The medical practitioner may then advance the endoscope distally to further view other portions as desired. After the visualization is complete, the medical practitioner withdraws the endoscope by moving it proximally until it is completely removed from the biological lumen of the patient.

FIG. 27 illustrates a schematic anatomical section of a medical scoping device fitting of the present application in the course of a medical scoping procedure. FIG. 27 shows insertion of the scoping device into the colon 90 of an individual undergoing an endoscopic procedure. The protuberances, one shown as 86, is flattened and moving radially inward toward the bottom 84 of the fitting 87 (shown as a cap) as the fitting enters the colon and the protuberances 86 are compressed by the colon wall. The fitting 87 covers the endoscope 80. At the top 82 of the fitting, there is an opening for the camera of the endoscope as it approaches a fold 88 in the colon.

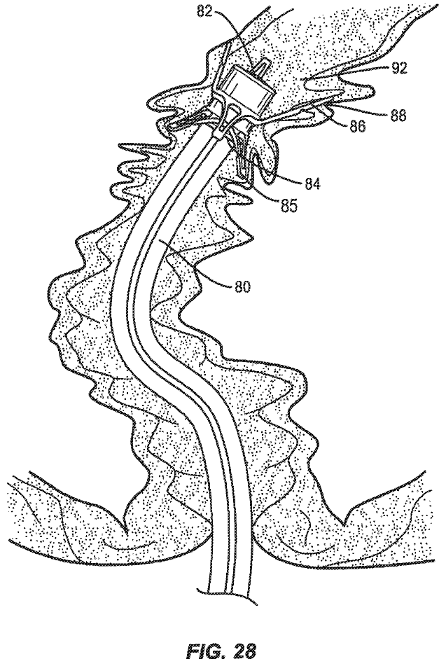

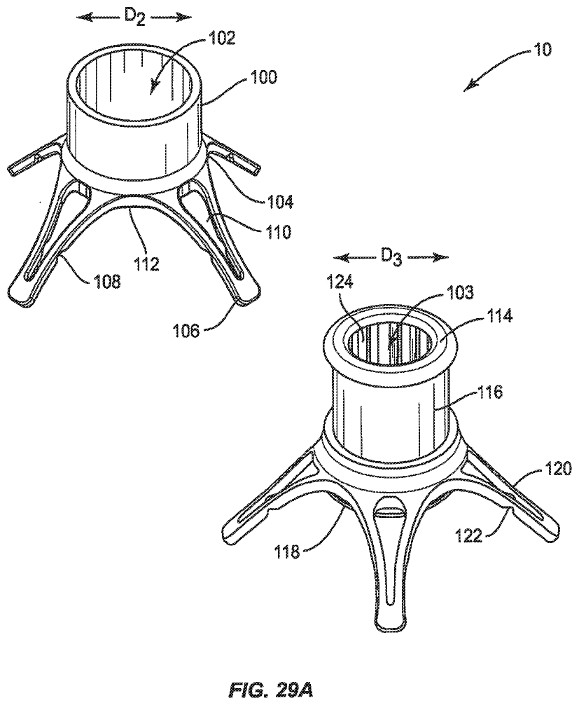

FIG. 28 illustrates a schematic anatomical section of a medical scoping device fitting of the present application shown in FIG. 27 in the course of a medical scoping procedure, where the protuberance 86 moves radially outward as the endoscope 80 is withdrawn from the colon and protuberances unfold the fold 88 in the colon to improve visualization out of the top 82 of the fitting of the colon lining 92, alternatively this can be accomplished by air suction causing the colon wall to collapse or wrap around the fitting. The bottom 84 of the fitting has other protuberances attached to it such as protuberance 85, which is in the resting position.

In some embodiments, a method of performing an endoscopy is provided, the method comprising disposing an endoscope cap on a distal end of an endoscope, the cap comprising a body defining a longitudinal axis, the body having an interior having an opening to receive an endoscope along the longitudinal axis, the body having a cylindrical portion comprising protuberances being spaced apart and circumferentially arrayed with respect to one another and extending from the cylindrical portion, each of the protuberances having an outer edge to engage tissue, wherein (i) the interior comprises a plurality of raised surfaces disposed thereon; and/or (ii) each protuberance having a bottom surface having a plurality of raised surfaces disposed thereon; and inserting the distal end of the endoscope into a biological lumen to move the protuberances radially inward relative to the body of the cap; and moving the endoscope proximally in the biological lumen for a distance to move the protuberances radially outward relative to the body of the cap.

### Method of Making the Fitting

In some embodiments, fitting 10 may be made by injection molding, compression molding, blow molding, thermoforming, die pressing, slip casting, electrochemical machining, laser cutting, water-jet machining, electrophoretic deposition, powder injection molding, sand casting, shell mold casting, plaster-mold casting, investment casting, vacuum casting, permanent-mold casting, slush casting, pressure casting, die casting, centrifugal casting, squeeze casting, rolling, forging, swaging, extrusion, shearing, spinning, or combinations thereof.

In some embodiments, the fitting may be formed by 3D printing. Instructions in the form of schematics encompassing any of the embodiments disclosed herein may be given to a computer to be carried out by a 3D printer. An elastomeric material, such as a silicone-based elastomer may be fed into a reservoir to be used to form the fitting. In some embodiments, the components of the fitting may be color coded to signify various physical properties. For example, different colors may be used to differentiate between varying amounts of friction or flexibility between components. Once the material is chosen, an elastomeric material is deposited over a flat fabrication platform one layer at a time. Once a first layer is deposited, a second layer is deposited on top of the first layer. The process is repeated as necessary to create the fitting to the specifications enumerated in the instructions.

Another form of manufacturing the fitting involves casting the elastomeric material in a mold. The elastomeric material can take on the shape of the mold such as, crescent, quadrilateral, rectangular, cylindrical, plug, or any other shape. Additionally, the surface of the mold may be smooth or may include raised features or indentations, for example indentations to create the recesses or notches, to impart features to the fitting. Features from the mold can be imparted to the fitting as the elastomeric material in the mold is dried. In particular aspects, a roughened or friction engaging surface can be formed on the upper surface and/or the lower surface of the fitting body. In some embodiments, protuberances or raised portions can be imparted on the upper surface and/or the lower surface from the mold.

### Kits

The disclosed fitting and endoscope may be sterilizable. In various embodiments, one or more components of the fitting and/or endoscope may be sterilizable by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In some embodiments, the one or more components of the fitting may be packaged in a moisture resistant package and then terminally sterilized by gamma irradiation. In use, the medical practitioner performing the endoscopic procedure removes the one or all components from the sterile package for use.

In various embodiments, electron beam (e-beam) radiation may be used to sterilize one or more components of the fitting. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

Other methods may also be used to sterilize the fitting and/or one or more components of the fitting, including, but not limited to, gas sterilization, such as, for example, with ethylene oxide or steam sterilization.

In various embodiments, a kit is provided comprising the components of the fitting and an endoscope. The kit may include additional components along with the fitting, such as additional sets of protuberances. The kit may include the fitting in a first compartment. A second compartment may include an endoscope. A third compartment may include additional sets of protuberances, as well as an instruction booklet, which may include a chart that shows how to install and use the fitting. A cover of the kit may include illustrations of the installation and endoscopic procedure and a clear plastic cover may be placed over the compartments to maintain sterility.

### EXAMPLES

In Vivo Animal Model Testing

Users were asked questions that related to specific design characteristics of endoscope fittings/caps. Their feedback determined which design characteristics were considered most critical. During testing on porcine subjects, users switched to a “blinded” study to avoid bias. The fittings were placed on the endoscope and into the colon before the testing user took control of the procedure. Users answered questions relating to attachment, intubation, withdrawal and removal from the endoscope on a number scale from 1-5 with higher numbers equating to a more positive performance and lower numbers equating to a more negative performance. A number awarded of 1 equated to failed or not usable, a number awarded of 2 equated to needing improvement, a number awarded of 3 equated to satisfactory/usable, a number awarded of 4 equated to better than expected, and a number awarded of 5 equated to excellent. Critical characteristics were combined into 4 designs to create a variety of models.

Based on the users' feedback, key characteristics of the fittings/caps were concluded. The key characteristics included 1. the fittings/caps should not cause any more trauma than seen during a typical colonoscopy procedure, 2. the fittings/caps should manipulate the folds during withdrawal, 3. the more slim the profile, the better, 4. removal from the endoscope should not cause hand strain, and 5. the user should be able to see protuberances/fingers for a positive visual feedback.

Relating to the conclusions above, the users' concluded that the design should be atraumatic and not cause more trauma to the colon than a typical colonoscopy procedure. Further, a 70 Durometer hardness scored best during testing. A slim profile was desired and the more slim the profile, the better. Further, during endoscope removal, it was determined that the endoscope should be removed without hand strain and fittings/caps having a 70 Durometer scored the best. Visual feedback was important and it was determined that a user should be able to see protuberances/fingers as it is a positive visual feedback. It was determined that treads on bottom of protuberances did not make a difference in fold manipulation, however, treads were a unique feature.

Based on the in vivo testing results, consolidated fittings/caps were formed which relate to FIGS. 38A-C, 39A-39C, 40A-40C and 41A-41C. These fittings/caps included characteristics that were considered desirable by the user which included 70 Durometer material to avoid trauma, protuberances/fingers made slightly longer to aid fold manipulation and improve finger-tip visibility, a slim profile, treads removed from one design and rounded into bumps on another to avoid trauma, and protuberances/fingers of dual-row design made in-line to avoid pushing debris.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

1-42. (canceled)

43. A fitting for an endoscope, the fitting comprising an elongated body defining a longitudinal axis, the body having an interior having an opening to receive an endoscope along the longitudinal axis, the interior having a plurality of ribs extending longitudinally in the interior, the body having a cylindrical portion comprising elongated flexible protuberances being spaced apart and circumferentially arrayed with respect to one another and extending from the cylindrical portion, each elongated flexible protuberance having a bottom surface to engage tissue.

44. A fitting for an endoscope according to claim 43, wherein each elongated flexible protuberance has an inner end and an outer edge, and each elongated flexible protuberance has a varied degree of flexibility from the inner end to the outer edge.

45. A fitting for an endoscope according to claim 43, wherein the bottom surface is a smooth surface.

46. A fitting for an endoscope according to claim 43, wherein the fitting comprises a material comprising a hardness of about 70 durometers.

47. A fitting for an endoscope according to claim 43, wherein the fitting comprises a tab extending from a proximal end of the fitting, the fitting and/or tab having a perforation line disposed on the elongated body and aligned along the longitudinal axis, the perforation line configured to facilitate tearing to remove the fitting from the endoscope.

48. A fitting for an endoscope according to claim 43, wherein the fitting comprises a tab extending from a proximal end of the fitting, the tab configured to remove the fitting from the endoscope.

49. A fitting for an endoscope according to claim 48, wherein the fitting or tab comprises a perforation line disposed on the elongated body and aligned along the longitudinal axis, the perforation line configured to facilitate tearing and removal of the fitting from the endoscope.

50. A fitting for an endoscope according to claim 48, wherein the fitting or tab comprises a first perforation line and a second perforation line disposed on the elongated body and aligned along the longitudinal axis, the first perforation line being parallel to the second perforation line.

51. A fitting for an endoscope according to claim 50, wherein the first perforation line and the second perforation line are disposed on opposing sides of a cavity defined from an outer surface of the elongated body.

52. A fitting for an endoscope according to claim 47, wherein the tab facilitates a single use of the fitting.

53. A fitting for an endoscope according to claim 47, wherein the tab is destructively removed from the endoscope.

54. A fitting for an endoscope according to claim 47, wherein the fitting comprises a first tab and a second tab, the second tab being disposed opposite the first tab.

55. A fitting for an endoscope according to claim 43, wherein the fitting comprises a tab comprising a first wall and a second wall disposed along the elongated body and the longitudinal axis, the first wall and the second wall having a thickness that is less than a thickness of the body, and the tab having a projection extending outwardly from a proximal end of the fitting, wherein the tab is peeled off the body to facilitate removal of the fitting from the endoscope.

56. A fitting for an endoscope according to claim 43, wherein the fitting or tab comprises a wall disposed along the elongated body and the longitudinal axis, the wall having a thickness that is less than a thickness of the body to facilitate removal of the fitting from the endoscope.

57. A fitting for an endoscope according to claim 43, wherein the fitting or tab comprises a first wall and a second wall disposed along the elongated body and the longitudinal axis, the first wall and the second wall having a thickness that is less than a thickness of the body to facilitate removal of the fitting from the endoscope

58. A fitting for an endoscope according to claim 55, wherein the first wall and the second wall are disposed on opposing sides of a cavity defined from an outer surface of the elongated body.

59. A fitting for an endoscope according to claim 55, wherein the tab facilitates a single use of the fitting.

60. A fitting for an endoscope according to claim 55, wherein the fitting comprises a first tab and a second tab, the second tab being disposed opposite the first tab.

61. A fitting for an endoscope according to claim 43, wherein the fitting further comprises a tube disposed on a distal end of the body.

62. A fitting for an endoscope according to claim 61, wherein the tube is transparent and is fused to the distal end of the body.

63-78. (canceled)
